(19) United States
(12) Patent Application Publication
(10) Pub. No.: US 2023/0232827 A1
(43) Pub. Date: Jul. 27, 2023

(54) METHOD FOR CONTROLLING SOYBEAN RUST FUNGUS RESISTANT TO QoI FUNGICIDES

(71) Applicant: SUMITOMO CHEMICAL COMPANY, LIMITED, Chuo-ku (JP)

(72) Inventors: Hidekatsu TOBITA, Takarazuka-shi (JP); Takaaki NAKANO, Kasai-shi (JP); Yoshihiko NOKURA, Takarazuka-shi (JP)

(73) Assignee: SUMITOMO CHEMICAL COMPANY, LIMITED, Chuo-ku (JP)

(21) Appl. No.: 18/002,574

(22) PCT Filed: Jun. 29, 2021
(86) PCT No.: PCT/JP2021/024493

§ 371 Date: Dec. 20, 2022

(30) Foreign Application Priority Data
Jun. 30, 2020 (JP) 2020-112467

(51) Int. Cl. A01N 43/38; A01P 3/00; A01N 43/54; A01N 33/24; A01N 43/56

(52) U.S. Cl. CPC A01N 43/38; A01P 3/00; A01N 43/54; A01N 33/24; A01N 43/56

(57) ABSTRACT

The present invention provides a method for controlling a soybean rust fungus having an amino acid substitution of F129L in a mitochondrial cytochrome b protein. A compound represented by formula (I)

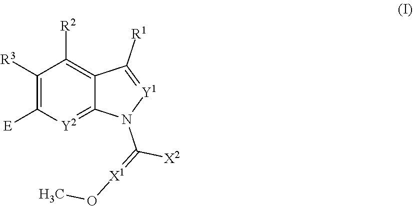

[wherein:
X¹ represents —C(H)═ or the like;
X² represents —C(O)OCH₃ or the like;
Y¹ represents —C(R⁴)═ or the like;
Y² represents —C(R⁵)═ or the like;
R¹, R², R³, R⁴, and R⁵ each represent a C1-C4 alkyl group or the like; and
E represents a C1-C6 chain hydrocarbon group or the like]
   can be used for controlling a soybean rust fungus having an amino acid substitution of F129L in a mitochondrial cytochrome b protein.

### TECHNICAL FIELD

This application claims the priority to and the benefit of Japanese Patent Application No. 2020-112467 filed on Jun. 30, 2020, the entire contents of which are incorporated herein by reference.

The present invention relates to a method and a compound for controlling a soybean rust fungus having an amino acid substitution of F129L in a mitochondrial cytochrome b protein.

### BACKGROUND ART

The spread of phytopathogenic fungi that shows acquired character being resistant to agricultural fungicides becomes a major problem. Under such circumstances, FRAC (Fungicide Resistance Action Committee) has been established as an organization that provides guidelines for acquiring a resistance to existing agricultural fungicides, and suppressing and delaying the spread of the fungi having the resistance acquired. A variety of information on phytopathogenic fungi that shows a resistance to agricultural fungicides is available on the FRAC-provided website (http://www.frac.info/).

It has been known that in the case of a phytopathogenic fungi, the main cause of acquiring a resistance is that a mutation of the phytopathogenic fungal gene encoding the target enzyme of the fungicide causes a partial substitution of amino acids in the target enzyme of the fungicides, which results in reducing the affinity between the fungicides and the target enzyme.

QoI fungicides are named as aliases a strobilurin fungicide, or a methoxyacrylate fungicide because of its characteristic structure. QoI fungicides are one group of agricultural fungicides that have been widely used to control phytopathogenic fungi including soybean rust fungus. QoI fungicides usually bind to the ubihydroquinone oxidation centers of cytochrome bc1 complex (electron transfer complex III) in mitochondria, and suppress a respiration of the phytopathogenic fungi, which results in killing the phytopathogenic fungi or stopping the growth of the same. The above-mentioned oxidation center is located outside the mitochondrial inner membrane (see NON-PATENT DOCUMENT 1).

It has been revealed by model studies in the laboratory before QoI fungicides were actually used extensively as agricultural fungicides that phytopathogenic fungi are subjected to a selection pressure by QoI fungicide, which results in easily generating the fungi having a resistance to a QoI fungicide that has acquired a gene mutation that causes a specific single amino acid substitution such as G143A in the cytochrome b gene of the target enzyme cytochrome bc1 complex (see NON-PATENT DOCUMENTS 2 to 4).

On the other hand, soybean rust fungus (scientific name: Phakopsora pachyrhizi) is a phytopathogenic fungus that causes damages to soybeans. Since QoI fungicides have been widely used for controlling soybean rust fungi, an emergence of soybean rust fungi showing a resistance to the QoI fungicides has been reported (see NON-PATENT DOCUMENT 5).

Regarding soybean rust fungi, a strain which has acquired a gene mutation causing a single amino acid substitution of F129L in the same cytochrome b gene has become a problem as a resistant fungus against QoI fungicides. The efficacy of the QoI fungicides conventionally used against soybean rust fungi, that is, pyribencarb, azoxystrobin, dimoxystrobin, metominostrobin, fluoxastrobin, kresoxim-methyl, and the others, has been reduced to the level of practical problems against said resistant fungi (see NON-PATENT DOCUMENT 6).

### CITATION LIST

### Non-Patent Document

NON-PATENT DOCUMENT 1: Sauter, “Modern Crop Protection Compounds”, Vol. 2, Wiley-VCH Verlag, 2007, p.457-495: Chapter 13.2, Strobilurins and other complex III inhibitors
NON-PATENT DOCUMENT 2: “Journal of Biological Chemistry”, 1989, Vol. 264, No. 24, p.14543-14548
NON-PATENT DOCUMENT 3: “Genetics”, 1991, Vol. 127, p.335-343
NON-PATENT DOCUMENT 4: “Current Genetics”, 2000, Vol. 38, p.148-155
NON-PATENT DOCUMENT 5: “Pest Management Science”, 2014, Vol. 70, No. 3, p.378-388
NON-PATENT DOCUMENT 6: “Pesq. agropec. bras.” (Brasilia), 2016, Vol. 51, No. 5, p.407-421

### SUMMARY OF THE INVENTION

### Problems to be Solved by the Invention

On the basis of these facts, an object of the present invention is to provide a method and a compound for controlling a soybean rust fungus having an amino acid substitution of F129L in a mitochondrial cytochrome b protein.

### Means to Solve Problems

The present invention provides the followings.

[1] A method for controlling a soybean rust fungus having an amino acid substitution of F129L in a mitochondrial cytochrome b protein, which comprises applying an effective amount of a compound represented by formula (I)

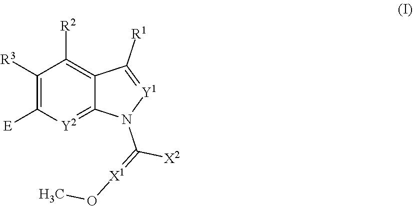

[wherein:

X¹ represents —C(H)═ or —N═;

X² represents —C(O)OCH₃, —C(O)NHCH₃, or a 5,6-dihydro-1,4,2-dioxazin-3-yl group;

Y¹ represents —C(R⁴)═ or —N═;

Y² represents —C(R⁵)═ or —N═;

R¹ and R⁴ are identical to or different from each other, and each represent a C1-C4 alkyl group, a C1-C4 alkoxy group, a C2-C4 alkylcarbonyl group, a C2-C4 alkoxycarbonyl group, a (C1-C3 alkylamino)carbonyl group, a (C2-C6 dialkylamino)carbonyl group {wherein said C1-C4 alkyl group, said C1-C4 alkoxy group, said C2-C4 alkylcarbonyl group, said C2-C4 alkoxycarbonyl group, said (C1-C3 alkylamino)carbonyl group, and said (C2-C6 dialkylamino)carbonyl group are optionally substituted with one or more halogen atom(s)}, a formyl group, an aminocarbonyl group, a cyano group, a nitro group, a halogen atom, a hydroxy group, or a hydrogen atom;

R², R³, and R⁵ are identical to or different from each other, and each represent a C1-C4 alkyl group, a C1-C4 alkoxy group {wherein said C1-C4 alkyl group and said C1-C4 alkoxy group are optionally substituted with one or more halogen atom(s)}, a cyano group, a nitro group, a halogen atom, a hydroxy group, or a hydrogen atom;

when Y¹ represents —C(R⁴)═, R¹ and R⁴ are optionally combined with the carbon atoms to which they are attached to form a C5-C6 nonaromatic hydrocarbon ring, a 5-6 membered nonaromatic heterocycle {wherein said C5-C6 nonaromatic hydrocarbon ring and said 5-6 membered nonaromatic heterocycle are optionally substituted with one or more substituent(s) selected from Group B}, a benzene ring, or a 5-6 membered aromatic heterocycle {wherein said benzene ring and said 5-6 membered aromatic heterocycle are optionally substituted with one or more substituent(s) selected from Group C};

E represents a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group D}, R⁹-L¹-CH₂—, R¹⁰R¹¹C═N—O—CH₂—, R¹²O—N═C(R¹³)—C(R¹⁴)═N—O—CH₂—, R¹⁵C(O)—C(R¹⁶)—N—O—CH₂—, R¹⁷R¹⁸N—C(S)—O—CH₂—, R¹⁹N═C(R²⁰—S—CH₂—, R²¹═C(SR²²)—S—CH₂—, R²³O—N═C(R²⁴)—S—CH₂—, R²⁵O—N═C(SR²⁶)—S—CH₂—, R²⁷O—N═C(R²⁸)—, R²⁹R³⁰C═N—N═C(R³¹)—, R³²R³³N—N═C(R³⁴)—, R³⁵—N═C(R³⁶)—, R³⁷SC(R³⁸)═N—, R³⁹SC(SR⁴⁰)═N—, R⁴¹L², R⁴³C(O)—, R⁴⁴OC(O)O—, R⁴⁵R⁴⁶NC(O)O—, R⁴⁷R⁴⁸NC(S)O—, R⁴⁹S(O)₂O—, R⁵⁰R⁵¹NS(O)₂O—, a cyano group, a nitro group, a hydroxy group, or a halogen atom;

L¹ and L² are identical to or different from each other, and each represent an oxygen atom or a sulfur atom;

R⁹ represents a C6-C10 aryl group or a 5-10 membered aromatic heterocyclic group {wherein said C6-C10 aryl group and said 5-10 membered aromatic heterocyclic group are optionally substituted with one or more substituent(s) selected from Group D};

R¹², R¹⁵, R¹⁷, R¹⁹, R²¹, R²³, R²⁵, R²⁹, R³², R³⁷, R³⁹, R⁴³, R⁴⁴, R⁴⁵, R⁴⁷, R⁴⁹, and R⁵⁰ are identical to or different from each other, and each represent a C1-C6 chain hydrocarbon group optionally substituted with one or more substituent(s) selected from Group F, a C3-C10 alicyclic hydrocarbon group optionally substituted with one or more substituent(s) selected from Group B, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group C};

R¹⁰, R²⁷, R³⁵, and R⁴¹ are identical to or different from each other, and each represent a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group D};

R¹¹, R¹³, R¹⁴, R¹⁶, R¹⁸, R²⁰, R²², R²⁴, R²⁶, R²⁸, R³⁰, R³¹, R³³, R³⁴, R³⁶, R³⁸, R⁴⁰, R⁴⁶, R⁴⁸, and R⁵¹ are identical to or different from each other, and each represent a C1-C3 chain hydrocarbon group optionally substituted with one or more halogen atom(s), a cyclopropyl group, or a hydrogen atom; R¹⁰ and R¹¹ are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}; and

R²⁹ and R³⁰ are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B};

Group A: a group consisting of a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C1-C4 alkoxy group, a C1-C4 alkylthio group {wherein said C1-C4 alkoxy group and said C1-C4 alkylthio group are optionally substituted with one or more substituent(s) selected from Group F}, a halogen atom, a cyano group, a nitro group, a hydroxy group, an oxo group, a thioxo group, a C6-C10 aryl group, and a 5-10 membered aromatic heterocyclic group {wherein said C6-C10 aryl group and said 5-10 membered aromatic heterocyclic group are optionally substituted with one or more substituent(s) selected from Group D};

Group B: a group consisting of an oxo group, a thioxo group, a C1-C3 chain hydrocarbon group, a C1-C3 alkoxy group {wherein said C1-C3 chain hydrocarbon group and said C1-C3 alkoxy group are optionally substituted with one or more halogen atom(s)}, a halogen atom, and a cyano group;

Group C: a group consisting of a C1-C6 chain hydrocarbon group, a C1-C6 alkoxy group, a C1-C6 alkylthio group {wherein said C1-C6 chain hydrocarbon group, said C1-C6 alkoxy group, and said C1-C6 alkylthio group are optionally substituted with one or more substituent(s) selected from Group F}, a C3-C6 cycloalkyl group {wherein said C3-C6 cycloalkyl group is optionally substituted with one or more substituent(s) selected from Group B}, a cyano group, a nitro group, a halogen atom, and a hydroxy group;

Group D: a group consisting of a C1-C6 chain hydrocarbon group, a C1-C6 alkoxy group, a C1-C6 alkylthio group, a C1-C6 alkylamino group, a C2-C8 dialkylamino group, a (C1-C6 alkyl)carbonyl group, a (C1-C6 alkoxy)carbonyl group, a (C1-C6 alkylamino)carbonyl group, a (C2-C8 dialkylamino)carbonyl group {wherein said C1-C6 chain hydrocarbon group, said C1-C6 alkoxy group, said C1-C6 alkylthio group, said C1-C6 alkylamino group, said C2-C8 dialkylamino group, said (C1-C6 alkyl)carbonyl group, said (C1-C6 alkoxy)carbonyl group, said (C1-C6 alkylamino)carbonyl group, and said (C2-C8 dialkylamino)carbonyl group are optionally substituted with one or more substituent(s) selected from Group F}, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group C}, a halogen atom, a cyano group, a nitro group, a hydroxy group, and an amino group;

Group F: a group consisting of a C3-C4 cycloalkyl group, a halogen atom, and a C1-C3 alkoxy group] or an N-oxide or an agriculturally acceptable salt thereof (hereinafter the compound represented by formula (I), or an N-oxide or an agriculturally acceptable salt thereof is referred to as “Present compound”) to a soybean or soil for cultivating a soybean.

[2] The method according to [1], wherein

X¹ represents —C(H)═; and

X² represents —C(O)OCH₃

in the compound represented by formula (I), or an N-oxide or an agriculturally acceptable salt thereof.
[3] The method according to [1] or [2], wherein

E represents a C1-C6 alkyl group optionally substituted with one or more substituent(s) selected from Group A, a C3-C6 cycloalkyl group {wherein said C3-C6 cycloalkyl group is optionally substituted with one or more substituent(s) selected from the group consisting of a C1-C3 alkyl group and a halogen atom}, a phenyl group, a 5-6 membered aromatic heterocyclic group {wherein said phenyl group and said 5-6 membered aromatic heterocyclic group are optionally substituted with one or more substituent(s) selected from Group D}, R⁹-L¹-CH₂—, R⁴¹L²-, or a halogen atom in the compound represented by formula (I), or an N-oxide or an agriculturally acceptable salt thereof.

[4] Use of the compound represented by formula (I), or an N-oxide or an agriculturally acceptable salt thereof as defined in any one of [1] to [3] for controlling a soybean rust fungus having an amino acid substitution of F129L in a mitochondrial cytochrome b protein.
[5] A compound represented by formula (II)

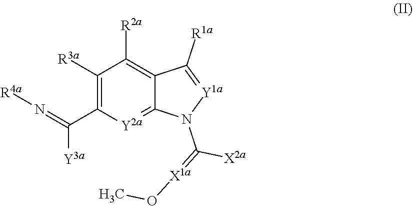

[wherein:

X^{1a} represents —C(H)═ or —N═;

X^{2a} represents —C(O)OCH₃, —C(O)NHCH₃, or a 5,6-dihydro-1,4,2-dioxazin-3-yl group;

Y^{1a} represents —C(R^{4a})═ or —N═;

Y^{2a} represents —C(R^{5a})═ or —N═;

R^{1a} and R^{4a} are identical to or different from each other, and each represent a C1-C4 alkyl group, a C1-C4 alkoxy group, a C2-C4 alkylcarbonyl group, a C2-C4 alkoxycarbonyl group, a (C1-C3 alkylamino)carbonyl group, a (C2-C6 dialkylamino)carbonyl group {wherein said C1-C4 alkyl group, said C1-C4 alkoxy group, said C2-C4 alkylcarbonyl group, said C2-C4 alkoxycarbonyl group, said (C1-C3 alkylamino)carbonyl group, and said (C2-C6 dialkylamino)carbonyl group are optionally substituted with one or more halogen atom(s)}, a formyl group, an aminocarbonyl group, a cyano group, a nitro group, a halogen atom, a hydroxy group, or a hydrogen atom;

R^{2a}, R^{3a}, and R^{5a} are identical to or different from each other, and each represent a C1-C4 alkyl group, a C1-C4 alkoxy group {wherein said C1-C4 alkyl group and said C1-C4 alkoxy group are optionally substituted with one or more halogen atom(s)}, a cyano group, a nitro group, a halogen atom, a hydroxy group, or a hydrogen atom;

when Y^{1a} represents —C(R^{4a})═, R^{1a} and R^{4a} are optionally combined with the carbon atoms to which they are attached to form a C5-C6 nonaromatic hydrocarbon ring, a 5-6 membered nonaromatic heterocycle {wherein said C5-C6 nonaromatic hydrocarbon ring and said 5-6 membered nonaromatic heterocycle are optionally substituted with one or more substituent(s) selected from Group B^{a}}, a benzene ring, or a 5-6 membered aromatic heterocycle {wherein said benzene ring and said 5-6 membered aromatic heterocycle are optionally substituted with one or more substituent(s) selected from Group C^{a}};

Y^{3a} represents a C1-C3 chain hydrocarbon group optionally substituted with one or more halogen atom(s), a cyclopropyl group, or a hydrogen atom;

Y^{4a} represents R^{27a}O—R^{29a}R^{30a}C—N—, R^{32a}R^{33a}N—, or R^{35a}—;

R^{29a} and R^{32a} are identical to or different from each other, and each represent a C1-C6 chain hydrocarbon group optionally substituted with one or more substituent(s) selected from Group F^{a}, a C3-C10 alicyclic hydrocarbon group optionally substituted with one or more substituent(s) selected from Group B^{a}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group C^{a}};

R^{27a} and R^{35a} are identical to or different from each other, and each represent a C1-C6 chain hydrocarbon group optionally substituted with one or more substituent(s) selected from Group A^{a}, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B^{a}}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group D^{a}};

R^{30a} and R^{33a} are identical to or different from each other, and each represent a C1-C3 chain hydrocarbon group optionally substituted with one or more halogen atom(s), a cyclopropyl group, or a hydrogen atom; and

R^{29a} and R^{30a} are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B^{a}};

Group A^{a}: a group consisting of a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B^{a}}, a C1-C4 alkoxy group, a C1-C4 alkylthio group {wherein said C1-C4 alkoxy group and said C1-C4 alkylthio group are optionally substituted with one or more substituent(s) selected from Group F^{a}}, a halogen atom, a cyano group, a nitro group, a hydroxy group, an oxo group, a thioxo group, a C6-C10 aryl group, and a 5-10 membered aromatic heterocyclic group {wherein said C6-C10 aryl group and said 5-10 membered aromatic heterocyclic group are optionally substituted with one or more substituent(s) selected from Group D^{a}};

Group B^{a}: a group consisting of an oxo group, a thioxo group, a C1-C3 chain hydrocarbon group, a C1-C3 alkoxy group {wherein said C1-C3 chain hydrocarbon group and said C1-C3 alkoxy group are optionally substituted with one or more halogen atom(s)}, a halogen atom, and a cyano group;

Group C^{a}: a group consisting of a C1-C6 chain hydrocarbon group, a C1-C6 alkoxy group, a C1-C6 alkylthio group {wherein said C1-C6 chain hydrocarbon group, said C1-C6 alkoxy group, and said C1-C6 alkylthio group are optionally substituted with one or more substituent(s) selected from Group F^{a}}, a C3-C6 cycloalkyl group {wherein said C3-C6 cycloalkyl group is optionally substituted with one or more substituent(s) selected from Group B^{a}}, a cyano group, a nitro group, a halogen atom, and a hydroxy group;

Group D^{a}: a group consisting of a C1-C6 chain hydrocarbon group, a C1-C6 alkoxy group, a C1-C6 alkylthio group, a C1-C6 alkylamino group, a C2-C8 dialkylamino group, a (C1-C6 alkyl)carbonyl group, a (C1-C6 alkoxy)carbonyl group, a (C1-C6 alkylamino)carbonyl group, a (C2-C8 dialkylamino)carbonyl group {wherein said C1-C6 chain hydrocarbon group, said C1-C6 alkoxy group, said C1-C6 alkylthio group, said C1-C6 alkylamino group, said C2-C8 dialkylamino group, said (C1-C6 alkyl)carbonyl group, said (C1-C6 alkoxy)carbonyl group, said (C1-C6 alkylamino)carbonyl group, and said (C2-C8 dialkylamino)carbonyl group are optionally substituted with one or more substituent(s) selected from Group F^{a}}, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B^{a}}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group C^{a}}, a halogen atom, a cyano group, a nitro group, a hydroxy group, and an amino group;

Group F^{a}: a group consisting of a C3-C4 cycloalkyl group, a halogen atom, and a C1-C3 alkoxy group] or an N-oxide thereof, or a salt thereof (hereinafter the compound represented by formula (II) or an N-oxide thereof, or a salt thereof is referred to as “Compound of the present invention”).

[6] The compound or an N-oxide thereof, or a salt thereof according to [5], wherein Y⁴a represents R^{27a}O—.
[7] An agrochemical composition comprising the compound or an N-oxide thereof, or a salt thereof according to [5] or
[6], and an inert carrier.
[8] A composition comprising one or more ingredient(s) selected from the group consisting of Group (a), Group (b), Group (c), and Group (d), and the compound or an N-oxide thereof, or a salt thereof according to [5] or [6]:

Group (a): a group consisting of insecticidal active ingredients, miticidal active ingredients, and nematicidal active ingredients;

Group (b): fungicidal active ingredients;

Group (c): plant growth regulatory ingredients;

Group (d): repellent ingredients.

[9] A method for controlling a soybean rust fungus having an amino acid substitution of F129L in a mitochondrial cytochrome b protein, which comprises applying an effective amount of the composition according to [7] or [8] to a soybean or soil for cultivating a soybean.
[10] Use of the composition according to [7] or [8] for controlling a soybean rust fungus having an amino acid substitution of F129L in a mitochondrial cytochrome b protein.
[11] A seed holding an effective amount of the compound or an N-oxide thereof, or a salt thereof according to [5] or [6], or an effective amount of the composition according to [7] or [8].

### Effect of Invention

According to the present invention, a soybean rust fungus having an amino acid substitution of F129L in a mitochondrial cytochrome b protein can be controlled.

### MODE FOR CARRYING OUT THE INVENTION

The substituents in the present invention are explained as follows.

The term of “halogen atom” represents a fluorine atom, a chlorine atom, a bromine atom, or an iodine atom.

When a substituent is substituted with two or more halogen atoms, these halogen atoms may be identical to or different from each other.

When a substituent is substituted with two or more groups or atoms selected from a specific group (for example, a group consisting of a C1-C3 alkyl group and a halogen atom), these groups or atoms may be identical to or different from each other.

The expression of “optionally substituted with one or more substituent(s) selected from Group X” (wherein X represents any one of A, B, C, D, F, A^{a}, B^{a}, C^{a}, D^{a}, and F^{a}) as described herein means that when two or more substituents are selected from Group X, these substituents may be identical to or different from each other.

The expression of “CX-CY” as described herein means that the number of carbon atom is X to Y. For example, the expression of “C1-C6” means that the number of carbon atom is 1 to 6.

The term of “chain hydrocarbon group” represents an alkyl group, an alkenyl group, or an alkynyl group.

Examples of the term of “alkyl group” include a methyl group, an ethyl group, a propyl group, an isopropyl group, a 1,1-dimethylpropyl group, a 1,2-dimethylpropyl group, a 1-ethylpropyl group, a butyl group, a sec-butyl group, a tert-butyl group, a pentyl group, and a hexyl group.

Examples of the term of “alkenyl group” include a vinyl group, a 1-propenyl group, a 2-propenyl group, a 1-methyl-1-propenyl group, a 1-methyl-2-propenyl group, a 1,2-dimethyl-1-propenyl group, a 1-ethyl-2-propenyl group, a 3-butenyl group, a 4-pentenyl group, and a 5-hexenyl group.

Examples of the term of “alkynyl group” include an ethynyl group, a 1-propynyl group, a 2-propynyl group, a 1-methyl-2-propynyl group, a 1,1-dimethyl-2-propynyl group, a 1-ethyl-2-propynyl group, a 2-butynyl group, a 4-pentynyl group, and a 5-hexynyl group.

Examples of the term of “alkoxy group” include a methoxy group, an ethoxy group, a propoxy group, an isopropoxy group, a butoxy group, a tert-butoxy group, a pentyloxy group, and a hexyloxy group.

Examples of the term of “alkylthio group” include a methylthio group, an ethylthio group, an isopropylthio group, and a hexylthio group.

Examples of the term of “alkylamino group” include a methylamino group, an ethylamino group, an isopropylamino group, and a hexylamino group.

Examples of the term of “dialkylamino group” include a dimethylamino group, a methylethylamino group, a diisopropylamino group, a methylheptylamino group, and a dibutylamino group.

Examples of the term of “(C1-C6 alkyl)carbonyl group” include an acetyl group, a propanoyl group, a 2-methylpropanoyl group, and a heptanoyl group.

Examples of the term of “C2-C4 alkylcarbonyl group” include an acetyl group, a propanoyl group, and a 2-methylpropanoyl group.

Examples of the term of “(C1-C6 alkoxy)carbonyl group” include a methoxycarbonyl group, an isopropoxycarbonyl group, and a hexyloxycarbonyl group.

Examples of the term of “C2-C4 alkoxycarbonyl group” include a methoxycarbonyl group and an isopropoxycarbonyl group.

Examples of the term of “(C1-C6 alkylamino)carbonyl group” include a methylaminocarbonyl group, an isopropylaminocarbonyl group, and a hexylaminocarbonyl group.

Examples of the term of “(C1-C3 alkylamino)carbonyl group” include a methylaminocarbonyl group and an isopropylaminocarbonyl group.

Examples of the term of “(C2-C8 dialkylamino)carbonyl group” include a dimethylaminocarbonyl group, a methylethylaminocarbonyl group, a diisopropylaminocarbonyl group, a methylheptylaminocarbonyl group, and a dibutylaminocarbonyl group.

Examples of the term of “(C2-C6 dialkylamino)carbonyl group” include a dimethylaminocarbonyl group, a methylethylaminocarbonyl group, and a diisopropylaminocarbonyl group.

Examples of the term of “alicyclic hydrocarbon group” include cycloalkyl groups or cycloalkenyl groups.

Examples of the term of “cycloalkyl group” include a cyclopropyl group, a cyclobutyl group, a cyclopentyl group, a cyclohexyl group, a bicyclo[6.5.0]nonyl group, and a bicyclo[6.6.0]decyl group.

Examples of the term of “cycloalkenyl group” include a cyclopentenyl group and a cyclohexenyl group. Also, said cycloalkenyl group may be fused to benzene ring(s), and examples thereof include an indanyl group and a tetrahydronaphthyl group.

Examples of the term of “nonaromatic hydrocarbon ring” include a cyclopentene ring, a cyclopentadiene ring, a cyclohexene ring, and a cyclohexadiene ring.

Examples of the term of “aryl group” include a phenyl group and a naphthyl group.

Examples of the term of “aromatic heterocyclic group” include 5 membered aromatic heterocyclic groups such as a pyrrolyl group, a furanyl group, a thienyl group, a pyrazolyl group, an imidazolyl group, a triazolyl group, a tetrazolyl group, an oxazolyl group, an isoxazolyl group, a triazolyl group, an isothiazolyl group, an oxadiazolyl group, and a thiadiazolyl group; 6 membered aromatic heterocyclic groups such as a pyridyl group, a pyridazinyl group, a pyrimidinyl group, a pyrazinyl group, a triazinyl group, and a tetrazinyl group; 9 membered aromatic heterocyclic groups such as an indazolyl group, an indolizinyl group, and an imidazopyridyl group; and 10 membered aromatic heterocyclic groups such as a quinolyl group, an isoquinolyl group, a quinazolinyl group, a naphthyridinyl group, and a benzopyranyl group.

Examples of the term of “nonaromatic heterocyclic group” include an aziridinyl group, an oxiranyl group, a thiiranyl group, an azetidinyl group, an oxetanyl group, a thietanyl group, a pyrrolidinyl group, a tetrahydrofuranyl group, a tetrahydrothienyl group, a pyrazolinyl group, a pyrazolidinyl group, an imidazolinyl group, an imidazolidinyl group, an oxazolinyl group, a thiazolinyl group, an oxazolidinyl group, a thiazolidinyl group, an isoxazolinyl group, an isoxazolidinyl group, an isothiazolinyl group, an isothiazolidinyl group, a dioxolanyl group, a dioxanyl group, a piperidyl group, a piperazinyl group, a morpholinyl group, a thiomorpholinyl group, a pyranyl group, a dihydropyranyl group, a tetrahydropyranyl group, a tetrahydrothiopyranyl group, an azepanyl group, an oxepanyl group, a thiepanyl group, a dihydrobenzofuranyl group, a 1,3-benzodioxolyl group, and a dihydrobenzopyranyl group.

The terms used herein are described as follows.

The term of “soybean rust fungus having an amino acid substitution of F129L in a mitochondrial cytochrome b protein” represents a soybean rust fungus (scientific name: Phakopsora pachyrhizi) wherein the mitochondrial cytochrome b gene encoding the mitochondrial cytochrome protein has a mutation, and an amino acid substitution of F129L is produced as a result from said mutation, thereby a resistance to QoI fungicides is developed.

The Present compounds and the Compounds of the present invention are QoI fungicide.

The Present compound and the Compound of the present invention may optionally have one or more stereoisomer(s). Examples of the stereoisomer(s) include enantiomers, diastereomers, atropisomers, and geometric isomers. The Present compound and the Compound of the present invention encompass each stereoisomer and mixtures of stereoisomers at any ratio.

Examples of the term of “agriculturally acceptable salt” of the Present compound, and “salt” of the compound represented by formula (II) or an N-oxide thereof include acid addition salts such as hydrochloride, sulfate, nitrate, phosphate, sulfonate, acetate, and benzoate.

Aspects of the Present compound include the following compounds.

[Aspect 1] The Present compound, wherein X¹ represents —C(H)═.
[Aspect 2] The Present compound, wherein X¹ represents —N═.
[Aspect 3] The Present compound, wherein X² represents —C(O)OCH₃.
[Aspect 4] The Present compound, wherein X² represents —C(O)NHCH₃.
[Aspect 5] The Present compound, wherein Y⁴ represents —C(R⁴)═.
[Aspect 6] The Present compound, wherein Y⁴ represents —N═.
[Aspect 7] The Present compound, wherein R⁴ represents a halogen atom or a hydrogen atom.
[Aspect 8] The Present compound, wherein R⁴ represents a chlorine atom or a hydrogen atom.
[Aspect 9] The Present compound, wherein R² represents a hydrogen atom.
[Aspect 10] The Present compound, wherein R⁴ and R² each represent a hydrogen atom.
[Aspect 11] The Present compound, wherein R³ represents a halogen atom or a hydrogen atom.
[Aspect 12] The Present compound, wherein R³ represents a fluorine atom or a hydrogen atom.
[Aspect 13] The Present compound, wherein

R⁴ and R² each represent a hydrogen atom; and

R³ represents a halogen atom or a hydrogen atom.

[Aspect 14] The Present compound, wherein Y² represents —C(R⁵)═.
[Aspect 15] The Present compound, wherein Y² represents —N═.
[Aspect 16] The Present compound, wherein

Y¹ represents —C(R⁴)═; and

Y² represents —C(R⁵)═.

[Aspect 17] The Present compound, wherein

Y¹ represents —C(R⁴)═; and

Y² represents —N═.

[Aspect 18] The Present compound, wherein

Y¹ represents —N═; and

Y² represents —C(R⁵)═.

[Aspect 19] The Present compound, wherein Y¹ and Y² each represent —N═.
[Aspect 20] The Present compound, wherein E represents a C1-C6 chain hydrocarbon group optionally substituted with one or more substituent(s) selected from Group A.
[Aspect 21] The Present compound, wherein E represents a C1-C6 alkyl group optionally substituted with one or more substituent(s) selected from Group A.
[Aspect 22] The Present compound, wherein E represents a C1-C6 alkyl group optionally substituted with one or more substituent(s) selected from the group consisting of a halogen atom, a phenyl group, and a cyclopropyl group.
[Aspect 23] The Present compound, wherein E represents a cyclopropylethynyl group.
[Aspect 24] The Present compound, wherein E represents a C3-C10 alicyclic hydrocarbon group optionally substituted with one or more substituent(s) selected from Group B.
[Aspect 25] The Present compound, wherein E represents a C3-C6 cycloalkyl group optionally substituted with one or more substituent(s) selected from the group consisting of a C1-C3 alkyl group and a halogen atom.
[Aspect 26] The Present compound, wherein E represents a 3-10 membered nonaromatic heterocyclic group optionally substituted with one or more substituent(s) selected from Group B.
[Aspect 27] The Present compound, wherein E represents a 3-10 membered nonaromatic heterocyclic group {wherein said 3-10 membered nonaromatic heterocyclic group are optionally substituted with one or more substituent(s) selected from the group consisting of a C1-C3 alkyl group and a halogen atom}.
[Aspect 28] The Present compound, wherein E represents a C6-C10 aryl group optionally substituted with one or more substituent(s) selected from Group D.
[Aspect 29] The Present compound, wherein E represents a phenyl group optionally substituted with one or more substituent(s) selected from Group D.
[Aspect 30] The Present compound, wherein E represents a phenyl group optionally substituted with one or more halogen atom(s).
[Aspect 31] The Present compound, wherein E represents a phenyl group.
[Aspect 32] The Present compound, wherein E represents a 5-10 membered aromatic heterocyclic group optionally substituted with one or more substituent(s) selected from Group D.
[Aspect 33] The Present compound, wherein E represents a 5-6 membered aromatic heterocyclic group optionally substituted with one or more substituent(s) selected from Group D.
[Aspect 34] The Present compound, wherein E represents a 5-6 membered aromatic heterocyclic group optionally substituted with one or more halogen atom(s).
[Aspect 35] The Present compound, wherein E represents R⁹-L¹-CH₂— or R⁴¹L²-.
[Aspect 36] The Present compound, wherein E represents R⁴¹L²-.
[Aspect 37] The Present compound, wherein E represents R¹⁰R¹¹C═N—O—CH₂—, R¹²O—N═C(R¹³)—C(R₁₄)═N—O—CH₂—, R¹⁵C(O)—C(R¹⁶)═N—O—CH₂—, R¹⁷R¹⁸N—C(S)—O—CH₂—, R¹⁹N═C(R²⁰)—S—CH₂—, R²¹N═C(SR²²)—S—CH₂—, R²³—N═C(R²⁴)—S—CH₂—, R²⁵O—N═C(SR²⁶)—S—CH₂—, R²⁷O—N═C(R²⁸)—, R²⁹R³⁰C═N—N═C(R³¹)—, R³²R³³N—N═C(R³⁴)—, R³⁵—N═C(R³⁶)—, R³⁷SC(R³⁸)═N—, R³⁹SC(SR⁴⁰═N—, R⁴³C(O)O—, R⁴⁴OC(O)—, R⁴⁵R⁴⁶NC(O)—, R⁴⁷R⁴⁸NC(S)O—, R⁴⁹S(O)₂O—, R⁵⁰R⁵¹NS(O)₂O—, a cyano group, a nitro group, or a hydroxy group.
[Aspect 38] The Present compound, wherein E represents R¹⁰R¹¹C═N—O—CH₂— or R²⁷O—N═C(R²⁸)—.
[Aspect 39] The Present compound, wherein E represents a halogen atom.
[Aspect 40] The Present compound, wherein E represents a bromine atom.
[Aspect 41] The Present compound, wherein E represents a C1-C6 alkyl group {wherein said C1-C6 alkyl group is optionally substituted with one or more substituent(s) selected from Group A}, a C3-C6 cycloalkyl group {wherein said C3-C6 cycloalkyl group is optionally substituted with one or more substituent(s) selected from the group consisting of a C1-C3 alkyl group and a halogen atom}, a 3-10 membered nonaromatic heterocyclic group {wherein said 3-10 membered nonaromatic heterocyclic group are optionally substituted with one or more substituent(s) selected from the group consisting of a C1-C3 alkyl group and a halogen atom}, a phenyl group, a 5-6 membered aromatic heterocyclic group {wherein said phenyl group and said 5-6 membered aromatic heterocyclic group are optionally substituted with one or more substituent(s) selected from Group D}, R⁴¹L²-, R¹⁰R¹¹C═N—O—CH₂—, R²⁷O—N═C(R²⁸)—, or a halogen atom.
[Aspect 42] The Present compound, wherein E represents a C1-C6 alkyl group {wherein said C1-C6 alkyl group is optionally substituted with one or more substituent(s) selected from the group consisting of a halogen atom, a phenyl group, and a cyclopropyl group}, a phenyl group, a 5-6 membered aromatic heterocyclic group {wherein said phenyl group and said 5-6 membered aromatic heterocyclic group are optionally substituted with one or more halogen atom(s)}, or a bromine atom.
[Aspect 43] The Present compound, wherein E represents a bromine atom, a cyclopropylethynyl group, or a phenyl group (wherein said phenyl group is optionally substituted with one or more halogen atom(s)).
[Aspect 44] The Present compound, wherein E represents a bromine atom, a cyclopropylethynyl group, or a phenyl group.
[Aspect 45] The compound according to the Aspect 41, wherein

X² represents —C(H)═;

X² represents —C(O)OCH₃;

Y² represents —C(H)═ or —N═;

R² represents a halogen atom or a hydrogen atom;

R² represents a hydrogen atom;

R³ represents a halogen atom or a hydrogen atom; and

Y² represents —C(H)═.

[Aspect 46] The compound according to the Aspect 42, wherein

X¹ represents —C(H)═;

X² represents —C(O)OCH₃;

Y¹ represents —C(H)═ or —N═;

R¹ represents a halogen atom or a hydrogen atom;

R² represents a hydrogen atom;

R³ represents a halogen atom or a hydrogen atom; and

Y² represents —C(H)═.

[Aspect 47] The compound according to the Aspect 43, wherein

X¹ represents —C(H)═;

X² represents —C(O)OCH₃;

R¹, R², and R⁴ each represent a hydrogen atom;

R³ represents a fluorine atom or a hydrogen atom;

when Y¹ represents —C(R⁴)═, R¹ and R⁴ are optionally combined with the carbon atoms to which they are attached to form a benzene ring; and

Y² represents —C(H)═.

[Aspect 48] The compound according to the Aspect 44, wherein

X¹ represents —C(H)═;

X² represents —C(O)OCH₃;

R¹, R², and R⁴ each represent a hydrogen atom;

R³ represents a fluorine atom or a hydrogen atom;

when Y¹ represents —C(R⁴)═, R¹ and R⁴ are optionally combined with the carbon atoms to which they are attached to form a benzene ring; and

Y² represents —C(H)═.

[Aspect 49] The Present compound, wherein E represents R²⁷O—N═C(R²⁸)—, R²⁹R³⁰C═N—N═C(R³¹)—, R³²R³³N—N═C(R³⁴)—, or R³⁵—N═C(R³⁶)—.
[Aspect 50] The Present compound, wherein E represents R²⁷O—N═C(R²⁸)—.
[Aspect 51] The Present compound, wherein

X² represents —C(O)OCH₃ or —C(O)NHCH₃;

Y² represents —C(H)═ or —N═;

R¹ and R⁴ are identical to or different from each other, and each represent a C1-C4 alkyl group optionally substituted with one or more halogen atom(s), a cyano group, a halogen atom, or a hydrogen atom;

R² and R³ are identical to or different from each other, and each represent a C1-C4 alkyl group optionally substituted with one or more halogen atom(s), a halogen atom, or a hydrogen atom; and

when Y¹ represents —C(R⁴)═, R¹ and R⁴ are optionally combined with the carbon atoms to which they are attached to form a benzene ring.

[Aspect 52] The Present compound, wherein

X¹ represents —C(H)═;

X² represents —C(O)OCH₃;

Y¹ represents —C(R⁴)═;

Y² represents —C(H)═;

R¹ represents a cyano group, a halogen atom, or a hydrogen atom;

R⁴ represents a methyl group, a cyano group, or a hydrogen atom;

R² represents a hydrogen atom; and

R³ represents a methyl group, a halogen atom, or a hydrogen atom.

[Aspect 53] The compound according to any one of the Aspect 51, the Aspect 52, or the Present compound, wherein

E represents a C1-C3 chain hydrocarbon group optionally substituted with one or more substituent(s) selected from Group A², a cyclopropyl group, a phenyl group, a pyridyl group, a thienyl group, a benzothienyl group {wherein said phenyl group, said pyridyl group, said thienyl group, and said benzothienyl group are optionally substituted with one or more substituent(s) selected from Group D²}, R²⁷O—N═C(R²⁸)—, R⁴¹O—, a hydroxy group, or a halogen atom;

R²⁷ represents a C1-C4 alkyl group optionally substituted with one or more substituent(s) selected from Group A³;

R²⁸ represents a methyl group or a hydrogen atom; and

R⁴¹ represents a C1-C3 chain hydrocarbon group optionally substituted with one or more halogen atom(s), a phenyl group, or a pyridyl group {wherein said phenyl group and said pyridyl group are optionally substituted with one or more substituent(s) selected from Group D²};

Group A²: a group consisting of a cyclopropyl group and a halogen atom;

Group A³: a group consisting of a phenyl group and a halogen atom;

Group D²: a group consisting of a C1-C3 alkyl group, a C1-C3 alkoxy group {wherein said C1-C3 alkyl group and said C1-C3 alkoxy group are optionally substituted with one or more halogen atom(s)}, a cyclopropyl group optionally substituted with one or more cyano group(s), and a halogen atom.

[Aspect 54] The compound according to any one of the Aspect 51, the Aspect 52, or the Present compound, wherein

E represents a C2 chain hydrocarbon group optionally substituted with one or more substituent(s) selected from Group A², a cyclopropyl group, a phenyl group, a thienyl group, a benzothienyl group {wherein said phenyl group, said pyridyl group, said thienyl group, and said benzothienyl group are optionally substituted with one or more substituent(s) selected from Group D³}, R²⁷O—N═C(R²⁸)—, or R⁴¹O—;

R²⁷ represents a C1-C4 alkyl group optionally substituted with one or more substituent(s) selected from Group A³;

R²⁸ represents a methyl group or a hydrogen atom; and

R⁴¹ represents a C1-C3 chain hydrocarbon group optionally substituted with one or more halogen atom(s), a phenyl group, or a pyridyl group {wherein said phenyl group and said pyridyl group are optionally substituted with one or more substituent(s) selected from Group D³};

Group D³: a group consisting of a methyl group, a methoxy group {wherein said methyl group and said methoxy group are optionally substituted with one or more halogen atom(s)}, and a halogen atom.

[Aspect 55] The compound according to any one of the Aspect 51, the Aspect 52, or the Present compound, wherein

E represents a C2 chain hydrocarbon group optionally substituted with a cyclopropyl group, a cyclopropyl group, a phenyl group, a thienyl group, a benzothienyl group {wherein said phenyl group, said thienyl group, and said benzothienyl group are optionally substituted with one or more substituent(s) selected from Group D⁴}, R²⁷O—N═C(R²⁸)—, or R⁴¹O—;

R²⁷ represents a C1-C4 alkyl group optionally substituted with one or more substituent(s) selected from Group A³;

R²⁸ represents a methyl group or a hydrogen atom; and

R⁴¹ represents a C1-C3 alkyl group optionally substituted with one or more halogen atom(s), a phenyl group, or a pyridyl group {wherein said phenyl group and said pyridyl group are optionally substituted with one or more substituent(s) selected from Group D⁴};

Group D⁴: a group consisting of a methyl group optionally substituted with one or more halogen atom(s), a methoxy group, and a halogen atom.

[Aspect 56] The compound according to any one of the Aspect 51, the Aspect 52, or the Present compound, wherein E represents a C1-C3 chain hydrocarbon group optionally substituted with one or more substituent(s) selected from Group A² or a cyclopropyl group.
[Aspect 57] The compound according to any one of the Aspect 51, the Aspect 52, or the Present compound, wherein E represents a C1-C3 chain hydrocarbon group optionally substituted with one or more substituent(s) selected from Group A².
[Aspect 58] The compound according to any one of the Aspect 51, the Aspect 52, or the Present compound, wherein E represents a C2 chain hydrocarbon group optionally substituted with a cyclopropyl group or a cyclopropyl group.
[Aspect 59] The compound according to any one of the Aspect 51, the Aspect 52, or the Present compound, wherein E represents a phenyl group, a thienyl group, or a benzothienyl group {wherein said phenyl group, said thienyl group, and said benzothienyl group are optionally substituted with one or more substituent(s) selected from Group D²}.
[Aspect 60] The compound according to any one of the Aspect 51, the Aspect 52, or the Present compound, wherein E represents a phenyl group, a thienyl group {wherein said phenyl group and said thienyl group are optionally substituted with one or more substituent(s) selected from Group D⁴}, or benzothienyl group.
[Aspect 61] The compound according to any one of the Aspect 51, the Aspect 52, or the Present compound, wherein

E represents R²⁷O—N═C(R²⁸)—;

R²⁷ represents a C1-C4 alkyl group optionally substituted with one or more substituent(s) selected from Group A³; and

R²⁸ represents a methyl group or a hydrogen atom.

[Aspect 62] The compound according to any one of the Aspect 51, the Aspect 52, or the Present compound, wherein

E represents R⁴¹O—; and

R⁴¹ represents a C1-C3 chain hydrocarbon group optionally substituted with one or more halogen atom(s), a phenyl group, or a pyridyl group {wherein said phenyl group and said pyridyl group are optionally substituted with one or more substituent(s) selected from Group D²}.

Aspects of the Compound of the present invention include the following compounds.

[Aspect C1] The Compound of the present invention, wherein X^{1a} represents —C(H)═.
[Aspect C2] The Compound of the present invention, wherein X^{1a} represents —N═.
[Aspect C3] The Compound of the present invention, wherein X^{2a} represents —C(O)OCH₃.
[Aspect C4] The Compound of the present invention, wherein X^{2a} represents —C(O)NHCH₃.
[Aspect C5] The Compound of the present invention, wherein Y^{1a} represents —C(R⁴)═.
[Aspect C6] The Compound of the present invention, wherein Y^{1a} represents —N═.
[Aspect C7] The Compound of the present invention, wherein R^{1a} represents a halogen atom or a hydrogen atom.
[Aspect C8] The Compound of the present invention, wherein R^{1a} represents a chlorine atom or a hydrogen atom.
[Aspect C9] The Compound of the present invention, wherein R^{2a} represents a hydrogen atom.
[Aspect C10] The Compound of the present invention, wherein R^{1a} and R^{2a} each represent a hydrogen atom.
[Aspect C11] The Compound of the present invention, wherein R^{3a} represents a halogen atom or a hydrogen atom.
[Aspect C12] The Compound of the present invention, wherein R^{3a} represents a fluorine atom or a hydrogen atom.
[Aspect C13] The Compound of the present invention, wherein

R^{1a} and R^{2a} each represent a hydrogen atom; and

R^{3a} represents a halogen atom or a hydrogen atom.

[Aspect C14] The Compound of the present invention, wherein Y^{2a} represents —C(R^{5a})═.
[Aspect C15] The Compound of the present invention, wherein Y^{2a} represents —N═.
[Aspect C16] The Compound of the present invention, wherein

Y^{1a} represents —C(R^{4a})═; and

Y^{2a} represents —C(R^{5a})═.

[Aspect C17] The Compound of the present invention, wherein

Y^{1a} represents —C(R^{4a})═; and

Y^{2a} represents —N═.

[Aspect C18] The Compound of the present invention, wherein

Y^{1a} represents —N═; and

Y^{2a} represents —C(R^{5a})═.

[Aspect C19] The Compound of the present invention, wherein

Y^{1a} and Y^{2a} each represent —N═.

[Aspect C20] The Compound of the present invention, wherein Y⁴a represents R^{27a}O—.
[Aspect C21] The Compound of the present invention, wherein

X^{1a} represents —C(H)═;

X^{2a} represents —C(O)OCH₃;

Y^{1a} represents —C(R^{4a})═;

Y^{2a} represents —C(R^{5a})═; and

Y⁴a represents R^{27a}O—.

[Aspect C22] The Compound of the present invention, wherein

X^{1a} represents —C(H)═; and

X^{2a} represents —C(O) OCH₃.

[Aspect C23] The Compound of the present invention, wherein

Y^{1a} represents —C(H)═;

Y^{2a} represents —C(H)═; and

R^{1a} represents a hydrogen atom.

[Aspect C24] The compound according to the Aspect C23, wherein

Y^{1a} represents —C(H)═;

Y^{2a} represents —C(H)═; and

R^{1a} represents a hydrogen atom.

[Aspect C25] The Compound of the present invention, wherein R^{2a} and R^{3a} are identical to or different from each other, and each represent a C1-C4 alkyl group or a hydrogen atom.
[Aspect C26] The Compound of the present invention, wherein R^{2a} and R^{3a} are identical to or different from each other, and each represent a methyl group or a hydrogen atom.
[Aspect C27] The Compound of the present invention, wherein

R^{2a} represents a hydrogen atom; and

R^{3a} represents a methyl group or a hydrogen atom.

[Aspect C28] The compound according to the Aspect C22, wherein R^{2a} and R^{3a} are identical to or different from each other, and each represent a C1-C4 alkyl group or a hydrogen atom.
[Aspect C29] The compound according to the Aspect C22, wherein R^{2a} and R^{3a} are identical to or different from each other, and each represent a methyl group or a hydrogen atom.
[Aspect C30] The compound according to the Aspect C22, wherein

R^{2a} represents a hydrogen atom; and

R^{3a} represents a methyl group or a hydrogen atom.

[Aspect C31] The compound according to the Aspect C23, wherein R^{2a} and R^{3a} are identical to or different from each other, and each represent a C1-C4 alkyl group or a hydrogen atom.
[Aspect C32] The compound according to the Aspect C23, wherein R^{2a} and R^{3a} are identical to or different from each other, and each represent a methyl group or a hydrogen atom.
[Aspect C33] The compound according to the Aspect C23, wherein

R^{2a} represents a hydrogen atom; and

R^{3a} represents a methyl group or a hydrogen atom.

[Aspect C34] The compound according to the Aspect C24, wherein R^{2a} and R^{3a} are identical to or different from each other, and each represent a C1-C4 alkyl group or a hydrogen atom.
[Aspect C35] The compound according to the Aspect C24, wherein R^{2a} and R^{3a} are identical to or different from each other, and each represent a methyl group or a hydrogen atom.
[Aspect C36] The compound according to the Aspect C24, wherein

R^{2a} represents a hydrogen atom; and

R^{3a} represents a methyl group or a hydrogen atom.

[Aspect C37] The compound according to any one of the Aspects C22 to C36 or the Compound of the present invention, wherein

Y^{3a} represents a C1-C3 chain hydrocarbon group optionally substituted with one or more halogen atom(s) or a hydrogen atom;

Y⁴a represents R^{27a}O—; and

R^{27a} represents a C1-C6 chain hydrocarbon group optionally substituted with one or more substituent(s) selected from Group A^{a2};

Group A^{a2}: a group consisting of a phenyl group optionally substituted with one or more halogen atom(s), and a halogen atom.
[Aspect C38] The compound according to any one of the Aspects C22 to C36 or the Compound of the present invention, wherein

Y^{3a} represents a methyl group optionally substituted with one or more halogen atom(s) or a hydrogen atom;

Y⁴a represents R^{27a}O—; and

R^{27a} represents a C1-C6 chain hydrocarbon group optionally substituted with one or more substituent(s) selected from Group A^{a2}.

[Aspect C39] The compound according to any one of the Aspects C22 to C36 or the Compound of the present invention, wherein

Y^{3a} represents a methyl group or a hydrogen atom;

Y⁴a represents R^{27a}O—; and

R^{27a} represents a C1-C4 alkyl group optionally substituted with one or more substituent(s) selected from Group A^{a3};

Group A^{a3}: a group consisting of a phenyl group and a halogen atom.

Next, Production methods for the Present compounds and the Compounds of the present invention are described.

### Production Method A

A compound represented by formula (A1) (hereinafter referred to as “Compound (A1)”) may be prepared by reacting a compound represented by formula (B1) (hereinafter referred to as “Compound (B1)”) with a compound represented by formula (M1) (hereinafter referred to as “Compound (M1)”) in the presence of a palladium catalyst and a base.

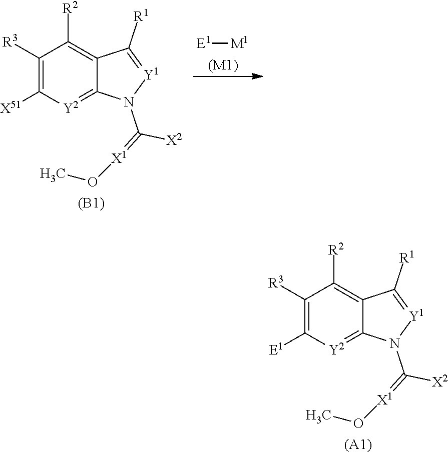

[wherein E¹ represents a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group D}; M¹ represents B(OH)₂ or a 4,4,5,5-tetramethyl-1,3,2-dioxaborolan-2-yl group; X⁵¹ represents a chlorine atom, a bromine atom, an iodine atom, or triflyloxy group; and the other symbols are the same as defined above.]

The reaction is usually carried out in a solvent. Examples of the solvent to be used in the reaction include hydrocarbons such as hexane, toluene, and xylene (hereinafter collectively referred to as “hydrocarbons”); ethers such as methyl tert-butyl ether (hereinafter referred to as “MTBE”), tetrahydrofuran, and dimethoxyethane (hereinafter referred to as “DME”) (hereinafter collectively referred to as “ethers”); halogenated hydrocarbons such as chloroform and chlorobenzene (hereinafter collectively referred to as “halogenated hydrocarbons”); amides such as dimethylformamide (hereinafter referred to as “DMF”) and N-methylpyrrolidone (hereinafter collectively referred to as “amides”); esters such as methyl acetate and ethyl acetate (hereinafter collectively referred to as “esters”); nitriles such as acetonitrile and propionitrile (hereinafter collectively referred to as “nitriles”); water; and mixtures of two or more of them.

Examples of the palladium catalyst to be used in the reaction include [1,1′-bis(diphenylphosphino) ferrocene]palladium(II) dichloride.

Examples of the base to be used in the reaction include organic bases such as triethylamine and pyridine (hereinafter collectively referred to as “organic bases”); alkali metal carbonates such as sodium carbonate and potassium carbonate (hereinafter collectively referred to as “alkali metal carbonates”); alkali metal hydrogen carbonates such as sodium hydrogen carbonate and potassium hydrogen carbonate (hereinafter collectively referred to as “alkali metal hydrogen carbonates”); sodium fluoride; and tripotassium phosphate.

In the reaction, the Compound (M1) is usually used at a ratio of 1 to 10 mol, the palladium catalyst is usually used at a ratio of 0.01 to 1 mol, and the base is usually used at a ratio of 1 to 10 mol, relative to 1 mol of the Compound (B1).

The reaction temperature is usually within the range of 0 to 150° C. The reaction time is usually within the range of 0.1 to 120 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A1).

The Compound (B1) and the Compound (M1) are known or may be prepared according to known methods.

### Production Method B

The Compound (A1) may also be prepared by reacting a compound represented by formula (B2) (hereinafter referred to as “Compound (B2)”) with a compound represented by formula (M2) (hereinafter referred to as “Compound (M2)”) in the presence of a palladium catalyst and a base.

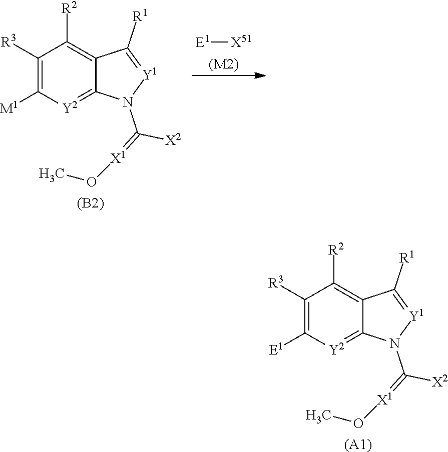

[wherein the symbols are the same as defined above.]

The reaction may be carried out according to the Production method A by using the Compound (M2) instead of the Compound (B1), and using the Compound (B2) instead of the Compound (M1).

The Compound (B2) and the Compound (M2) are known or may be prepared according to known methods.

### Production Method C

A compound represented by formula (A2) (hereinafter referred to as “Compound (A2)”) may be prepared by reacting the Compound (B1) with a compound represented by formula (M3) (hereinafter referred to as “Compound (M3)”) in the presence of a metal catalyst and a base.

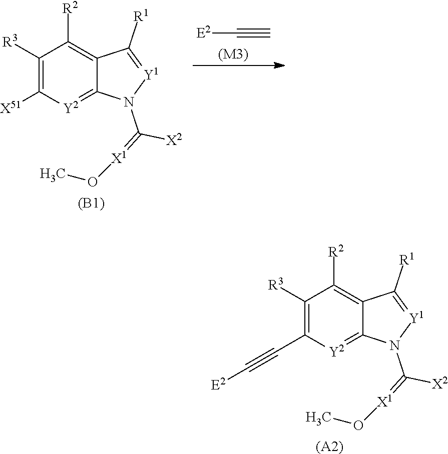

[wherein E² represents a C1-C4 chain hydrocarbon group optionally substituted with one or more substituent(s) selected from Group A; and the other symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the metal catalyst to be used in the reaction include bis(triphenylphosphine)palladium(II) dichloride and copper(I) iodide.

Examples of the base to be used in the reaction include organic bases.

In the reaction, the Compound (M3) is usually used at a ratio of 1 to 10 mol, the metal catalyst is usually used at a ratio of 0.01 to 1 mol, and the base is usually used at a ratio of 1 to 10 mol, relative to 1 mol of the Compound (B1).

The reaction temperature is usually within the range of 0 to 150° C. The reaction time is usually within the range of 0.1 to 120 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A2).

The Compound (M3) is known or may be prepared according to known method(s).

### Production Method D

A compound represented by formula (A3) (hereinafter referred to as “Compound (A3)”) may be prepared by reacting a compound represented by formula (B3) (hereinafter referred to as “Compound (B3)”) with a compound represented by formula (M4) (hereinafter referred to as “Compound (M4)”) or a salt thereof.

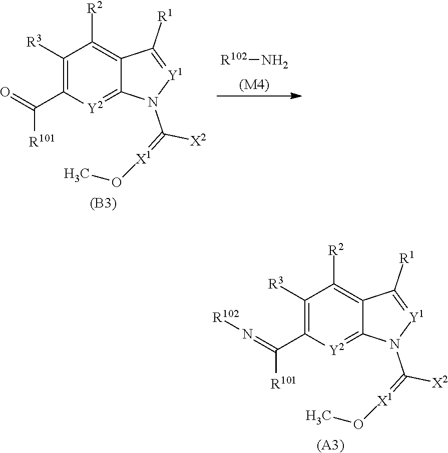

[wherein the combination of R¹⁰¹ and R¹⁰² represents a combination wherein R¹⁰¹ represents R²⁸ and R¹⁰² represents R²⁷O—, a combination wherein R¹⁰¹ represents R³¹ and R¹⁰² represents R²⁹R³⁰C═N—, a combination wherein R¹⁰¹ represents R³⁴ and R¹⁰² represents R³²R³³N—, or a combination wherein R¹⁰¹ represents R³⁶ and R¹⁰² represents R³⁵; and the other symbols are the same as defined above.]

Examples of the salt of the Compound (M4) include hydrochloride and sulfate.

The reaction is usually carried out in a solvent. Examples of the solvent to be used in the reaction include hydrocarbons; ethers; halogenated hydrocarbons; amides; esters; nitriles; alcohol such as methanol and ethanol (hereinafter collectively referred to as “alcohols”); and mixtures of two or more of them.

In the reaction, a base may be used as needed.

Examples of the base to be used in the reaction include organic bases, alkali metal carbonates, alkali metal hydrogen carbonates, sodium hydride, and tripotassium phosphate.

When a base is used in the reaction, the base is usually used at a ratio of 1 to 10 mol relative to 1 mol of the Compound (B3).

In the reaction, the Compound (M4) is usually used at a ratio of 1 to 10 mol relative to 1 mol of the Compound (B3).

The reaction temperature is usually within the range of 0 to 150° C. The reaction time is usually within the range of 0.1 to 120 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A3).

The Compound (M4) is known or may be prepared according to known method(s).

Production method E A compound represented by formula (A4) (hereinafter referred to as “Compound (A4)”) may be prepared by reacting a compound represented by formula (B4) (hereinafter referred to as “Compound (B4)”) with a compound represented by formula (M5) (hereinafter referred to as “Compound (M5)”) in the presence of a base.

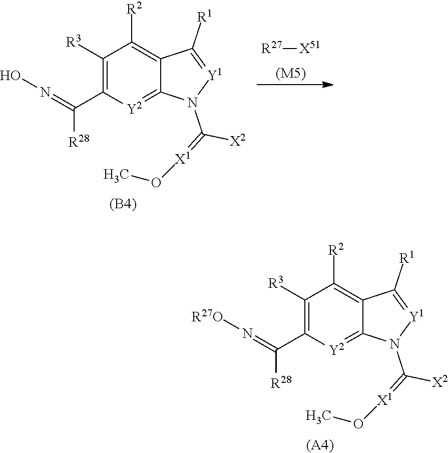

[wherein the symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, a metal catalyst and/or a ligand may be used as needed.

Examples of the metal catalyst include copper catalysts such as copper(I) iodide, copper(I) bromide, copper(I) chloride, copper(I) oxide, copper(I) trifluoromethanesulfonate benzene complex, tetrakis(acetonitrile)copper(I) hexafluorophosphate, and copper(I) 2-thiophenecarboxylate; and nickel catalysts such as bis(cyclooctadiene)nickel(0) and nickel(II) chloride. When a metal catalyst is used in the reaction, the metal catalyst is usually used at a ratio of 0.01 to 1 mol relative to 1 mol of the Compound (B4).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethylethylenediamine, and N,N-dimethylglycine hydrochloride. When a ligand is used in the reaction, the ligand is usually used at a ratio of 0.01 to 1 mol relative to 1 mol of the Compound (B4).

In the reaction, the Compound (M5) is usually used at a ratio of 1 to 10 mol, and the base is usually used at a ratio of 1 to 10 mol, relative to 1 mol of the Compound (B4).

The reaction temperature is usually within the range of −20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A4).

The Compound (M5) is known or may be prepared according to known method(s).

### Production Method F

A compound represented by formula (A5) (hereinafter referred to as “Compound (A5)”) may be prepared by reacting a compound represented by formula (B5) (hereinafter referred to as “Compound (B5)”) with a compound represented by formula (M6) (hereinafter referred to as “Compound (M6)”) in the presence of a base.

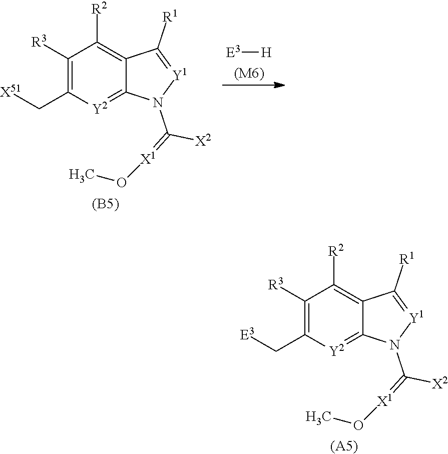

[wherein E³ represents R⁹-L¹-, R¹⁰R¹¹C═N—O—, R¹²O—N═C(R¹³)—C(R¹⁴)═N—O—, R¹⁵C(O)—C(R¹⁶)═N—O—, R¹⁷R¹⁸N—C(S)—O—, R¹⁹N═C(R²⁰)—S—, R²¹N═C(SR²²)—S—, R²³O—N═C(R²⁴)—S—, or R²⁵O—N═C(SR²⁶)—S—; and the other symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, the Compound (M6) is usually used at a ratio of 1 to 10 mol, and the base is usually used at a ratio of 1 to 10 mol, relative to 1 mol of the Compound (B5).

The reaction temperature is usually within the range of −20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A5).

The Compound (M6) is known or may be prepared according to known method(s).

### Production Method G

A compound represented by formula (A6) (hereinafter referred to as “Compound (A6)”) may be prepared by reacting a compound represented by formula (B6) (hereinafter referred to as “Compound (B6)”) with a compound represented by formula (M7) (hereinafter referred to as “Compound (M7)”) in the presence of a phosphine and an azodiester.

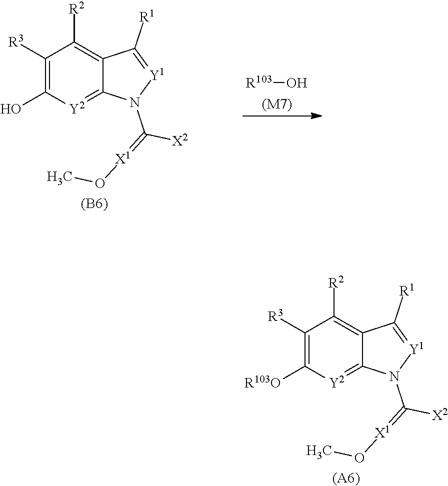

[wherein R¹⁰³ represents a C1-C6 chain hydrocarbon group optionally substituted with one or more substituent(s) selected from Group A or a C3-C10 alicyclic hydrocarbon group optionally substituted with one or more substituent(s) selected from Group B; and the other symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the phosphine include triphenylphosphine and trimethylphosphine.

Examples of the azodiester include diethyl azodicarboxylate, diisopropyl azodicarboxylate, and bis(2-methoxyethyl) azodicarboxylate.

In the reaction, the Compound (M7) is usually used at a ratio of 1 to 10 mol, the phosphine is usually used at a ratio of 1 to 10 mol, and the azodiester is usually used at a ratio of 1 to 10 mol, relative to 1 mol of the Compound (B6).

The reaction temperature is usually within the range of 0 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A6).

The Compound (M7) is known or may be prepared according to known method(s).

### Production Method H

A compound represented by formula (A7) (hereinafter referred to as “Compound (A7)”) may be prepared by reacting a compound represented by formula (B7) (hereinafter referred to as “Compound (B7)”) with a compound represented by formula (M8) (hereinafter referred to as “Compound (M8)”) in the presence of a base.

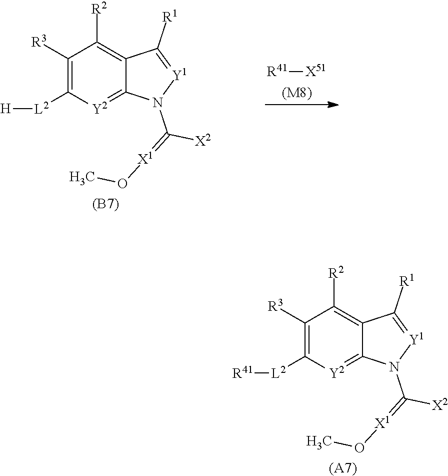

[wherein the symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, a metal catalyst and/or a ligand may be used as needed.

Examples of the metal catalyst include copper catalysts such as copper(I) iodide, copper(I) bromide, copper(I) chloride, copper(I) oxide, copper(I) trifluoromethanesulfonate benzene complex, tetrakis(acetonitrile)copper(I) hexafluorophosphate, and copper(I) 2-thiophenecarboxylate; and nickel catalysts such as bis(cyclooctadiene)nickel(0) and nickel(II) chloride. When a metal catalyst is used in the reaction, the metal catalyst is usually used at a ratio of 0.01 to 1 mol relative to 1 mol of the Compound (B7).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2˜dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethylethylenediamine, and N,N-dimethylglycine hydrochloride. When a ligand is used in the reaction, the ligand is usually used at a ratio of 0.01 to 1 mol relative to 1 mol of the Compound (B7).

In the reaction, the Compound (M8) is usually used at a ratio of 1 to 10 mol, and the base is usually used at a ratio of 1 to 10 mol, relative to 1 mol of the Compound (B7).

The reaction temperature is usually within the range of −20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A7).

The Compound (B7) and the Compound (M8) are known or may be prepared according to known method(s).

### Production Method I

The Compound (A7) may also be prepared by reacting the Compound (B1) with a compound represented by formula (M9) (hereinafter referred to as “Compound (M9)”) in the presence of a base.

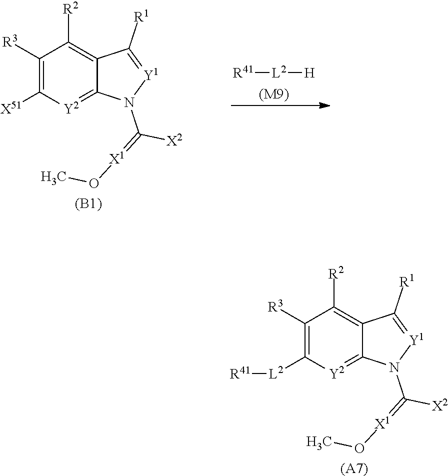

[wherein the symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, a metal catalyst and/or a ligand may be used as needed.

Examples of the metal catalyst include copper catalysts such as copper(I) iodide, copper(I) bromide, copper(I) chloride, copper(I) oxide, copper(I) trifluoromethanesulfonate benzene complex, tetrakis(acetonitrile)copper(I) hexafluorophosphate, and copper(I) 2-thiophenecarboxylate; and nickel catalysts such as bis(cyclooctadiene)nickel(0) and nickel(II) chloride. When a metal catalyst is used in the reaction, the metal catalyst is usually used at a ratio of 0.01 to 1 mol relative to 1 mol of the Compound (B1).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethylethylenediamine, and N,N-dimethylglycine hydrochloride. When a ligand is used in the reaction, the ligand is usually used at a ratio of 0.01 to 1 mol relative to 1 mol of the Compound (B1).

In the reaction, the Compound (M9) is usually used at a ratio of 1 to 10 mol, and the base is usually used at a ratio of 1 to 10 mol, relative to 1 mol of the Compound (B1).

The reaction temperature is usually within the range of −20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A7).

The Compound (M9) is known or may be prepared according to known method(s).

### Production Method J

A compound represented by formula (A8) (hereinafter referred to as “Compound (A8)”) may be prepared by reacting the Compound (B6) with a compound represented by formula (M10) (hereinafter referred to as “Compound (M10)”) in the presence of a base.

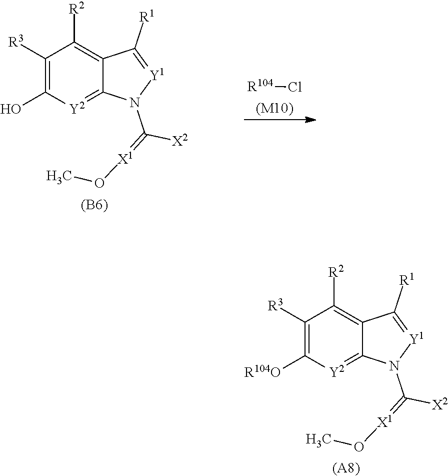

[wherein R¹⁰⁴ represents R⁴³C(O)—, R⁴⁴OC(O)—, R⁴⁵R⁴⁶NC(O)—, R⁴⁷R⁴⁸NC(S)—, R⁴⁹S(O)₂—, or R⁵⁰R⁵¹NS(O)₂—; and the other symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, the Compound (M10) is usually used at a ratio of 1 to 10 mol, and the base is usually used at a ratio of 1 to 10 mol, relative to 1 mol of the Compound (B6).

The reaction temperature is usually within the range of −78 to 100° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A8).

The Compound (M10) is known or may be prepared according to known method(s).

### Production Method K

A compound represented by formula (A9) (hereinafter referred to as “Compound (A9)”) may be prepared by carrying out a step of reacting a compound represented by formula (B8) (hereinafter referred to as “Compound (B8)”) with a compound represented by formula (M11) (hereinafter referred to as “Compound (M11)”) in the presence of a base to give a compound represented by formula (B9) (hereinafter referred to as “Compound (B9)”) (hereinafter referred to as “Step (K-1)”), and a step of reacting the Compound (B9) with a compound represented by formula (M12) (hereinafter referred to as “Compound (M12)”) in the presence of a base (hereinafter referred to as “Step (K-2)”).

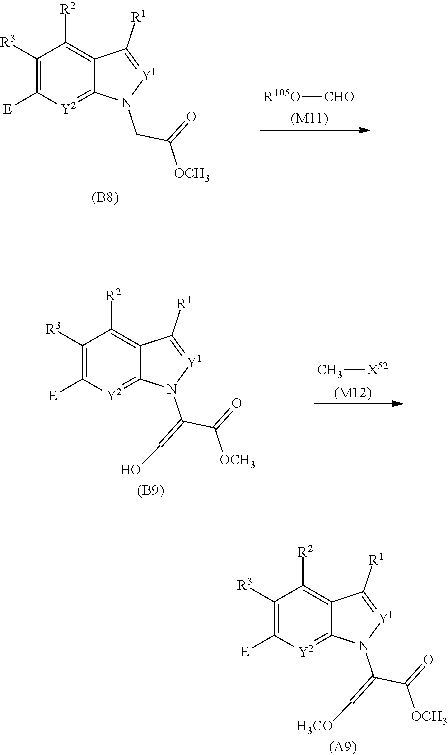

[wherein R¹⁰⁵ represents a C1-C4 alkyl group; X⁵² represents an iodine atom, a methoxysulfonyl group, a mesyloxy group, or a tosyloxy group; and the other symbols are the same as defined above.]

The Step (K-1) is usually carried out in a solvent. Examples of the solvent to be used in the reaction include ethers, amides, and mixtures of two or more of them.

Examples of the base to be used in the reaction include alkali metal hydrides such as sodium hydride.

In the reaction, the Compound (M11) is usually used at a ratio of 1 mol to 10 mol, and the base is usually used at a ratio of 0.5 mol to 5 mol, relative to 1 mol of the Compound (B8).

The reaction time is usually within the range of 5 minutes to 72 hours. The reaction temperature is usually within the range of −20° C. to 100° C.

When the reaction is completed, the reaction mixture may be subjected to a work-up such as adding water to the reaction mixture, extracting the resulting mixture with organic solvent(s), and drying and/or concentrating the resulting organic layer to give the Compound (B9).

The Compound (M11) is a commercially available compound or may be prepared according to known method(s).

The Step (K-2) is usually carried out in a solvent. Examples of the solvent to be used in the reaction include hydrocarbons, ethers, halogenated hydrocarbons, amides, esters, nitriles, and mixtures of two or more of them.

Examples of the base to be used in the reaction include organic bases, alkali metal carbonates, alkali metal hydrogen carbonates, sodium hydride, and mixtures of two or more of them.

In the reaction, the Compound (M12) is usually used at a ratio of 1 to 10 mol, and the base is usually used at a ratio of 1 to 20 mol, relative to 1 mol of the Compound (B9).

The reaction temperature is usually within the range of −20 to 100° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A9).

The Compound (M12) is known or may be prepared according to known method(s).

### Production Method L

A compound represented by formula (A10) (hereinafter referred to as “Compound (A10)”) may be prepared by carrying out a step of reacting the Compound (B8) with a compound represented by formula (M13) (hereinafter referred to as “Compound (M13)”) in the presence of a base to give a compound represented by formula (B10) (hereinafter referred to as “Compound (B10)”) (hereinafter referred to as “Step (L-1)”), and a step of reacting the Compound (B10) with the Compound (M12) in the presence of a base (hereinafter referred to as “Step (L-2)”).

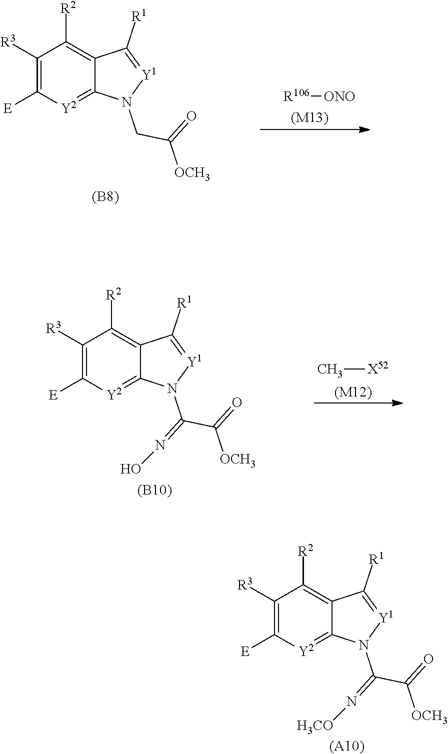

[wherein R¹⁰⁶ represents a tert-butyl group or an isopentyl group; and the other symbols are the same as defined above.]

The Step (L-1) is usually carried out in a solvent. Examples of the solvent to be used in the reaction include ethers, amides, alcohols, and mixtures of two or more of them.

Examples of the base to be used in the reaction include sodium hydride; and alkali metal alkoxides such as sodium methoxide, sodium ethoxide, and potassium t-butoxide.

In the reaction, the Compound (M13) is usually used at a ratio of 1 mol to 10 mol, and the base is usually used at a ratio of 1 mol to 5 mol, relative to 1 mol of the Compound (B8).

The reaction time is usually within the range of 5 minutes to 72 hours. The reaction temperature is usually within the range of −20° C. to 100° C.

When the reaction is completed, the reaction mixture may be subjected to a work-up such as adding water to the reaction mixture, extracting the resulting mixture with organic solvent(s), and drying and/or concentrating the resulting organic layer to give the Compound (B10).

The Compound (M13) is a commercially available compound.

The Step (L-2) may be carried out according to the Step (K-2) of the Production method K by using the Compound (B10) instead of the Compound (B9).

Production method M

A compound represented by formula (A12) (hereinafter referred to as “Compound (A12)”) may be prepared by reacting a compound represented by formula (A11) (hereinafter referred to as “Compound (A11)”) with methylamine.

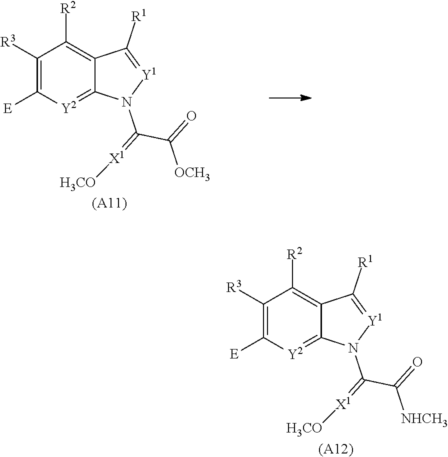

[wherein the symbols are the same as defined above.]

The reaction is usually carried out in a solvent. Examples of the solvent to be used in the reaction include alcohols, hydrocarbons, ethers, halogenated hydrocarbons, amides, esters, nitriles, and mixtures of two or more of them.

In the reaction, a base may be used as needed. Examples of the base to be used in the reaction include organic bases; alkali metal carbonates; alkali metal hydroxides such as sodium hydroxide and potassium hydroxide (hereinafter collectively referred to as “alkali metal hydroxides”); and sodium hydride. When a base is used in the reaction, the base is usually used at a ratio of 0.1 to 10 mol relative to 1 mol of the Compound (A11).

The methylamine is usually used as a solution. Examples of the solution of methylamine include a methanol solution and an aqueous solution.

In the reaction, the methylamine is usually used at a ratio of 1 to 100 mol relative to 1 mol of the Compound (A11).

The reaction temperature is usually within the range of −20 to 60° C. The reaction time is usually within the range of 0.1 to 120 hour(s).

When the reaction is completed, the reaction mixture may be subjected to a work-up such as mixing the reaction mixture with water, then extracting the resulting mixture with organic solvent(s), and drying and/or concentrating the resulting organic layer to isolate the Compound (A12).

### Production Method N

A compound represented by formula (A13) (hereinafter referred to as “Compound (A13)”) may be prepared by carrying out a step of reacting the Compound (A10) with hydroxylamine in the presence of a base to give a compound represented by formula (B11) (hereinafter referred to as “Compound (B11)”) (hereinafter referred to as “Step (N-1)”), and a step of reacting the Compound (B11) with a compound represented by formula (M14) (hereinafter referred to as “Compound (M14)”) in the presence of a base (hereinafter referred to as “Step (N-2)”).

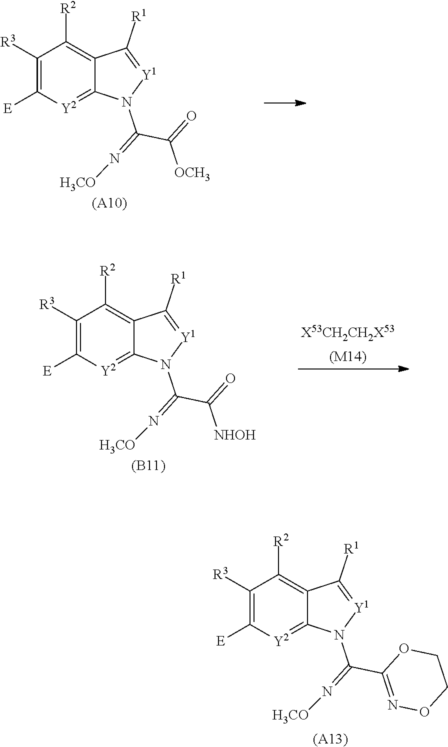

[wherein X⁵³ represents a chlorine atom, a bromine atom, or an iodine atom; and the other symbols are the same as defined above.]

The Step (N-1) may be carried out according to the Production method M by using the Compound (A10) instead of the Compound (A11), and using hydroxylamine instead of methylamine.

The Step (N-2) is usually carried out in a solvent. Examples of the solvent to be used in the reaction include hydrocarbons, ethers, halogenated hydrocarbons, amides, esters, nitriles, water, and mixtures of two or more of them.

Examples of the base include organic bases, alkali metal carbonates, alkali metal hydrogen carbonates, sodium hydride, and tripotassium phosphate.

In the reaction, the Compound (M14) is usually used at a ratio of 1 to 10 mol, and the base is usually used at a ratio of 1 to 10 mol, relative to 1 mol of the Compound (B11).

The reaction temperature is usually within the range of −20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A13).

The Compound (M14) is known or may be prepared according to known method(s).

### Production Method O

The Compound (A10) may also be prepared by a step of reacting a compound represented by formula (B12) (hereinafter referred to as “Compound (B12)”) with a compound represented by formula (M15) (hereinafter referred to as “Compound (M15)”) in the presence of a base to give the Compound (B10) (hereinafter referred to as “Step (O-1)”), and a step of reacting the Compound (B10) with the Compound (M12) in the presence of a base (hereinafter referred to as “Step (O-2)”). Also, the Compound (A10) may also be prepared by a step of reacting the Compound (B12) with a compound represented by formula (M16) (hereinafter referred to as “Compound (M16)”) in the presence of a base (hereinafter referred to as “Step (O-3)”).

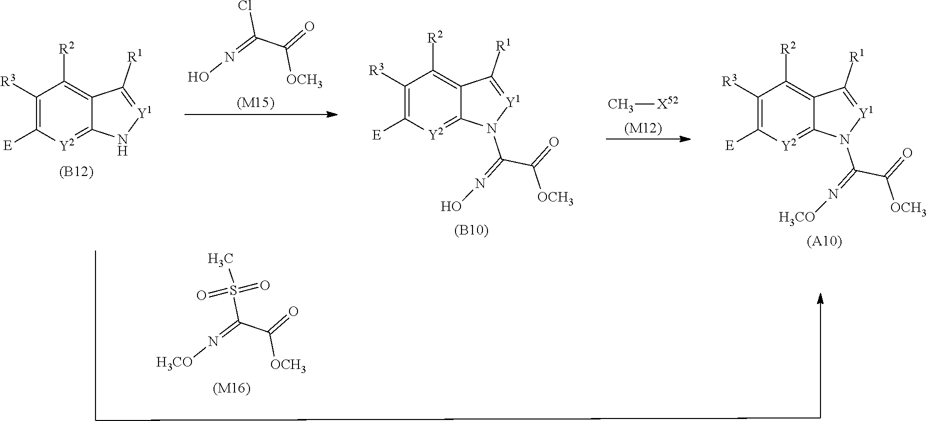

[wherein the symbols are the same as defined above.]

The Step (O-1) is usually carried out in a solvent. Examples of the solvent to be used in the reaction include hydrocarbons, ethers, halogenated hydrocarbons, amides, esters, nitriles, water, and mixtures of two or more of them.

Examples of the base include organic bases, alkali metal carbonates, alkali metal hydrogen carbonates, sodium hydride, and tripotassium phosphate.

In the reaction, the Compound (M15) is usually used at a ratio of 1 to 10 mol, and the base is usually used at a ratio of 1 to 10 mol, relative to 1 mol of the Compound (B12).

The reaction temperature is usually within the range of −20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (B10).

The Compound (B12) is known or may be prepared according to known method(s). The Compound (M15) is a known compound.

The Step (O-2) may be carried out according to the method described in the Step (L-2) of the Production method L.

The Step (O-3) is usually carried out in a solvent. Examples of the solvent to be used in the reaction include hydrocarbons, ethers, halogenated hydrocarbons, amides, esters, nitriles, water, and mixtures of two or more of them.

Examples of the base include organic bases, alkali metal carbonates, alkali metal hydrogen carbonates, sodium hydride, and tripotassium phosphate.

In the reaction, the Compound (M16) is usually used at a ratio of 1 to 10 mol, and the base is usually used at a ratio of 1 to 10 mol, relative to 1 mol of the Compound (B12).

The reaction temperature is usually within the range of −20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A10).

The Compound (M16) is a known compound.

### Production Method P

The N-oxide of the compound represented by formula (I) or the compound represented by formula (II) may be prepared by reacting the compound represented by formula (I) or the compound represented by formula (II) with an oxidizing agent. The reaction may be carried out according to the method described in, for example, US Patent Application Publication No. 2018/0009778 or WO 2016/121970 pamphlet.

### Reference Production Method a

A compound represented by formula (B65) (hereinafter referred to as “Compound (B65)”) may be prepared by reacting the Compound (B1) with bis(pinacolato)diboron in the presence of a base and a palladium catalyst.

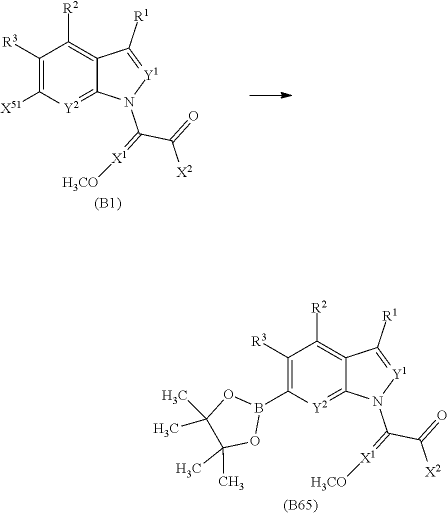

[wherein the symbols are the same as defined above.]

The reaction is usually carried out in a solvent. Examples of the solvent to be used in the reaction include hydrocarbons; ethers; halogenated hydrocarbons; amides; esters; sulfoxides such as dimethylsulfoxide (hereinafter referred to as “DMSO”) (hereinafter collectively referred to as “sulfoxides”); nitriles; and mixtures of two or more of them.

Examples of the base to be used in the reaction include organic bases, alkali metal carbonates, alkali metal hydrogen carbonates, and tripotassium phosphate.

Examples of the palladium catalyst include [1,1′-bis(diphenylphosphino) ferrocene]palladium(II) dichloride.

In the reaction, bis(pinacolato)diboron is usually used at a ratio of 1 to 5 mol, the base is usually used at a ratio of 1 to 5 mol, and the palladium catalyst is usually used at a ratio of 0.01 to 0.5 mol, relative to 1 mol of the Compound (B1).

The reaction temperature is usually within the range of 0 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (B65).

### Reference Production Method b

A compound represented by formula (B66) (hereinafter referred to as “Compound (B66)”) may be prepared by reacting the Compound (B1) with a compound represented by formula (M18) (hereinafter referred to as “Compound (M18)”).

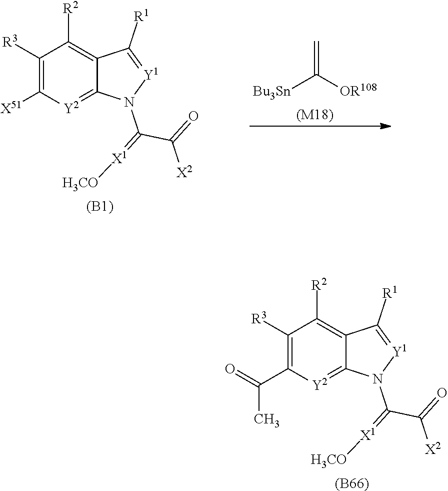

[wherein R¹⁰⁸ represents a methyl group or an ethyl group; and the other symbols are the same as defined above.]

The reaction may be carried out according to the method described in, for example, WO 2016/123253 pamphlet.

The Compound (M18) is a known compound.

### Reference Production Method c

A compound represented by formula (B67) (hereinafter referred to as “Compound (B67)”) may be prepared by reacting the Compound (B66) with hydroxylamine or a salt thereof.

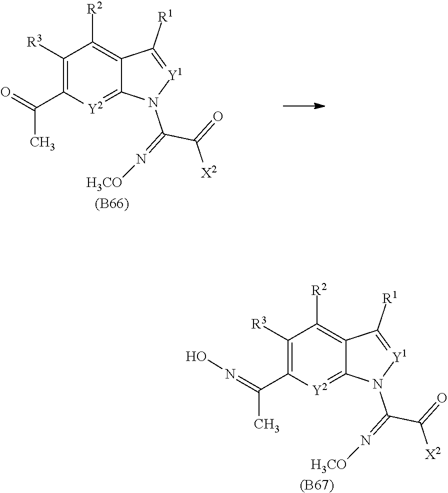

[wherein the symbols are the same as defined above.]

Examples of the salt of hydroxylamine include hydrochloride and sulfate.

The reaction may be carried out according to the Production method D by using the Compound (B66) instead of the Compound (B3), and using hydroxylamine or a salt thereof instead of the Compound (M4).

### Reference Production Method d

A compound represented by formula (B69) (hereinafter referred to as “Compound (B69)”) may be prepared by carrying out a step of reacting the Compound (B1) with N-formylsaccharin in the presence of a palladium catalyst, a ligand, triethylsilane, and a base to give a compound represented by formula (B68) (hereinafter referred to as “Compound (B68)”) (hereinafter referred to as “Step (d-1)”), a step of reacting the Compound (B68) with sodium borohydride to give the Compound (B5) (hereinafter referred to as “Step (d-2)”), and a step of reacting the Compound (B5) with carbon tetrachloride, carbon tetrabromide, or iodine in the presence of triphenylphosphine (hereinafter referred to as “Step (d-3)”).

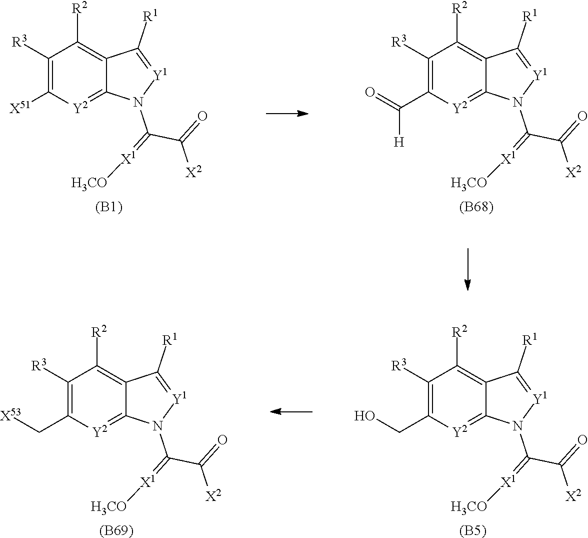

[wherein the symbols are the same as defined above.]

The Step (d-1) may be carried out according to the method described in Angew. Chem. Int. Ed., 2013, 52, 8611-8615 or the like.

The Step (d-2) may be carried out according to the method described in Chemistry-A European Journal, 2019, 25(15), 3950-3956 or the like.

The Step (d-3) may be carried out according to the method described in J. Org. Synth., 1974, 54, 63 or the like.

### Reference Production Method e

The Compound (B6) may be prepared by reacting the Compound (B2) with an oxidizing agent.

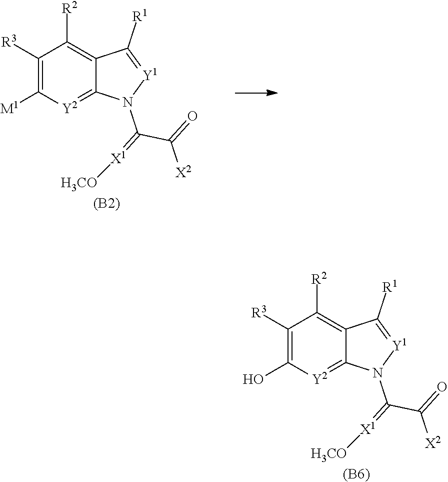

[wherein the symbols are the same as defined above.]

The reaction is usually carried out in a solvent. Examples of the solvent to be used in the reaction include hydrocarbons, ethers, halogenated hydrocarbons, amides, esters, nitriles, alcohols, water, and mixtures of two or more of them.

Examples of the oxidizing agent to be used in the reaction include m-chloroperoxybenzoic acid and hydrogen peroxide water.

When hydrogen peroxide water is used as the oxidizing agent, a base may be used as needed.

Examples of the base to be used in the reaction include alkali metal hydroxides.

When a base is used in the reaction, the base is usually used at a ratio of 0.1 to 5 mol relative to 1 mol of the Compound (B2).

In the reaction, the oxidizing agent is usually used at a ratio of 1 to 5 mol relative to 1 mol of the Compound (B2).

The reaction temperature is usually within the range of −20 to 120° C. The reaction time is usually within the range of 0.1 to 48 hour(s).

When the reaction is completed, the reaction mixture may be subjected to a work-up such as adding water and a reducing agent such as sodium thiosulfate to the reaction mixture, extracting the resulting mixture with organic solvent(s), and drying and/or concentrating the resulting organic layer to isolate the Compound (B6).

### Reference Production Method f

The Compound (B8) may be prepared by reacting the Compound (B12) with bromomethyl acetate or chloromethyl acetate in the presence of a base.

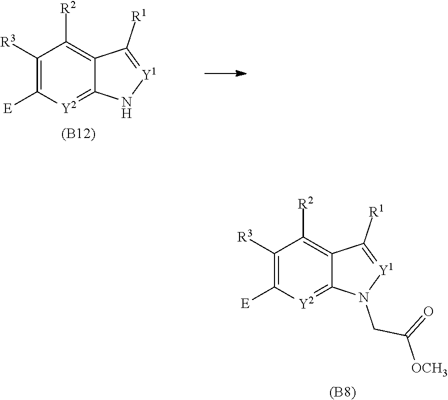

[wherein the symbols are the same as defined above.]

The reaction is usually carried out in a solvent. Examples of the solvent to be used in the reaction include hydrocarbons, ethers, halogenated hydrocarbons, amides, esters, nitriles, water, and mixtures of two or more of them.

Examples of the base include organic bases, alkali metal carbonates, alkali metal hydrogen carbonates, sodium hydride, and tripotassium phosphate.

In the reaction, bromomethyl acetate or chloromethyl acetate is usually used at a ratio of 1 to 10 mol, and the base is usually used at a ratio of 1 to 10 mol, relative to 1 mol of the Compound (B12).

The reaction temperature is usually within the range of −20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (B8).

The Compound of the present invention may be mixed with or used in combination with one or more ingredient(s) selected from the group consisting of the following Group (a), Group (b), Group (c), and Group (d) (hereinafter referred to as “Present ingredient”).

When the Compound of the present invention is mixed with or used in combination with the Present ingredient, they are used simultaneously, separately, or at time intervals with each other.

When the Compound of the present invention is used simultaneously with the Present ingredient, the Compound of the present invention and the Present ingredient may be contained in separate formulations or contained in one formulation.

One aspect of the present invention provides a composition comprising one or more ingredient(s) selected from the group consisting of Group (a), Group (b), Group (c), and Group (d) (i.e., the Present ingredient), and the Compound of the present invention (hereinafter referred to as “Composition A”).

Group (a) is a group consisting of acetylcholinesterase inhibitors (for example, carbamate insecticides and organophosphate insecticides), GABA-gated chloride channel blockers (for example, phenylpyrazole insecticides), sodium channel modulators (for example, pyrethroid insecticides), nicotinic acetylcholine receptor competitive modulators (for example, neonicotinoid insecticides), nicotinic acetylcholine receptor allosteric modulators, glutamate-gated chloride channel allosteric modulators (for example, macrolide insecticides), juvenile hormone mimics, multisite inhibitors, chordotonal organ TRPV channel modulators, mite growth inhibitors, microbial disruptors of insect midgut membranes, inhibitors of mitochondrial ATP synthase, uncouplers of oxidative phosphorylation, nicotinic acetylcholine receptor channel blockers (for example, nereistoxin insecticides), inhibitors of chitin biosynthesis, moulting disruptors, ecdysone receptor agonists, octopamine receptor agonists, mitochondrial complexes I, II, III, and IV electron transport inhibitors, voltage-dependent sodium channel blockers, inhibitors of acetyl CoA carboxylase, ryanodine receptor modulators (for example, diamide insecticides), chordotonal organ modulators, and microbial insecticides, and other insecticidal active ingredients, miticidal active ingredients, and nematicidal active ingredients. These ingredients are described in the classification on the basis of action mechanism by IRAC.

Group (b) is a group consisting of nucleic acids synthesis inhibitors (for example, phenylamide fungicides and acylamino acid fungicides), cell division and cytoskeleton inhibitors (for example, MBC fungicides), respiration inhibitors (for example, QoI fungicides and QiI fungicides), amino acids synthesis and protein synthesis inhibitors (for example, anilino-pyrimidine fungicides), signal transduction inhibitors, lipid synthesis and membrane synthesis inhibitors, sterol biosynthesis inhibitors (for example, DMI fungicides such as triazole fungicides), cell wall biosynthesis inhibitors, melanin synthesis inhibitors, plant defense inducers, fungicides with multi-site contact activity, microbial fungicides, and other fungicidal active ingredients. These ingredients are described in the classification on the basis of action mechanism by FRAC.

Group (c) is a group of plant growth regulatory ingredients (including mycorrhizal fungi and root nodule bacteria).

Group (d) is a group of repellent ingredients.

Hereinafter, examples of the combination of the Present ingredient and the Compound of the present invention are described. For example, “alanycarb+SX” indicates a combination of alanycarb and SX.

The abbreviation of “SX” indicates any one of the Compound of the present invention selected from the Compound groups SX1 to SX40 described in Examples. Also, all of the following Present ingredient are known ingredients, and may be obtained from commercially available formulations, or may be prepared by known methods. When the Present ingredient is a microorganism, it may also be available from a bacterial authority depository. Further, the number in parentheses represents the CAS RN (registered trademark).

Combinations of the Present ingredient in the above Group (a) and the Compound of the present invention:

abamectin+SX, acephate+SX, acequinocyl+SX, acetamiprid+SX, acetoprole+SX, acrinathrin+SX, acynonapyr+SX, afidopyropen+SX, afoxolaner+SX, alanycarb+SX, aldicarb+SX, allethrin+SX, alpha-cypermethrin+SX, alpha-endosulfan+SX, aluminium phosphide+SX, amitraz+SX, azadirachtin+SX, azamethiphos+SX, azinphos-ethyl+SX, azinphos-methyl+SX, azocyclotin+SX, bark of Celastrus angulatus+SX, bendiocarb+SX, benfluthrin+SX, benfuracarb+SX, bensultap+SX, benzoximate+SX, benzpyrimoxan+SX, beta-cyfluthrin+SX, beta-cypermethrin+SX, bifenazate+SX, bifenthrin+SX, bioallethrin+SX, bioresmethrin+SX, bistrifluron+SX, borax+SX, boric acid+SX, broflanilide+SX, bromopropylate+SX, buprofezin+SX, butocarboxim+SX, butoxycarboxim+SX, cadusafos+SX, calcium phosphide+SX, carbaryl+SX, carbofuran+SX, carbosulfan+SX, cartap hydrochloride+SX, cartap+SX, chinomethionat+SX, chlorantraniliprole+SX, chlordane+SX, chlorethoxyfos+SX, chlorfenapyr+SX, chlorfenvinphos+SX, chlorfluazuron+SX, chlormephos+SX, chloropicrin+SX, chlorpyrifos+SX, chlorpyrifos-methyl+SX, chromafenozide+SX, clofentezine+SX, clothianidin+SX, concanamycin A+SX, coumaphos+SX, cryolite+SX, cyanophos+SX, cyantraniliprole+SX, cyclaniliprole+SX, cyclobutrifluram+SX, cycloprothrin+SX, cycloxaprid+SX, cyenopyrafen+SX, cyetpyrafen+SX, cyflumetofen+SX, cyfluthrin+SX, cyhalodiamide+SX, cyhalothrin+SX, cyhexatin+SX, cypermethrin+SX, cyphenothrin+SX, cyproflanilide+SX, cyromazine+SX, dazomet+SX, deltamethrin+SX, demeton-S-methyl+SX, diafenthiuron+SX, diazinon+SX, dichlorvos+SX, dicloromezotiaz+SX, dicofol+SX, dicrotophos+SX, diflovidazin+SX, diflubenzuron+SX, dimefluthrin+SX, dimethoate+SX, dimethylvinphos+SX, dimpropyridaz+SX, dinotefuran+SX, disodium octaborate+SX, disulfoton+SX, DNOC (2-methyl-4,6-dinitrophenol)+SX, doramectin+SX, dried leaves of Dryopteris filix-mas+SX, emamectin-benzoate+SX, empenthrin+SX, endosulfan+SX, EPN (O-ethyl O-(4-nitrophenyl) phenylphosphonothioate)+SX, epsilon-metofluthrin+SX, epsilon-momfluorothrin+SX, esfenvalerate+SX, ethiofencarb+SX, ethion+SX, ethiprole+SX, ethoprophos+SX, etofenprox+SX, etoxazole+SX, extract of Artemisia absinthium+SX, extract of Azadirachta indica+SX, extract of Cassia nigricans+SX, extract of Clitoria ternatea+SX, extract of Symphytum officinale+SX, extract or simulated blend of Chenopodium ambrosioides+SX, extract of Tanacetum vulgare+SX, extract of Urtica dioica+SX, extract of Viscum album+SX, famphur+SX, fenamiphos+SX, fenazaquin+SX, fenbutatin oxide+SX, fenitrothion+SX, fenobucarb+SX, fenoxycarb+SX, fenpropathrin+SX, fenpyroximate+SX, fenthion+SX, fenvalerate+SX, fipronil+SX, flometoquin+SX, flonicamid+SX, fluacrypyrim+SX, fluazaindolizine+SX, fluazuron+SX, flubendiamide+SX, flucycloxuron+SX, flucythrinate+SX, fluensulfone+SX, flufenoprox+SX, flufenoxuron+SX, flufiprole+SX, flumethrin+SX, flupentiofenox+SX, flupyradifurone+SX, flupyrimin+SX, fluralaner+SX, fluvalinate+SX, fluxametamide+SX, formetanate+SX, fosthiazate+SX, furamethrin+SX, furathiocarb+SX, gamma-cyhalothrin+SX, GS-omega/kappa HXTX-Hvla peptide+SX, halfenprox+SX, halofenozide+SX, heptafluthrin+SX, heptenophos+SX, hexaflumuron+SX, hexythiazox+SX, potassium salt of hop beta acid+SX, hydramethylnon+SX, hydroprene+SX, imicyafos+SX, imidacloprid+SX, imidaclothiz+SX, imiprothrin+SX, indoxacarb+SX, isocycloseram+SX, isofenphos+SX, isoprocarb+SX, isopropyl-O-(methoxyaminothiophosphoryl) salicylate+SX, isoxathion+SX, ivermectin+SX, kadethrin+SX, kappa-tefluthrin+SX, kappa-bifenthrin+SX, kinoprene+SX, lambda-cyhalothrin+SX, lenoremycin+SX, lepimectin+SX, lime sulfur+SX, lotilaner+SX, lufenuron+SX, machine oil+SX, malathion+SX, mecarbam+SX, meperfluthrin+SX, metaflumizone+SX, metam+SX, methamidophos+SX, methidathion+SX, methiocarb+SX, methomyl+SX, methoprene+SX, methoxychlor+SX, methoxyfenozide+SX, methyl bromide+SX, metofluthrin+SX, metolcarb+SX, metoxadiazone+SX, mevinphos+SX, milbemectin+SX, milbemycin oxime+SX, momfluorothrin+SX, monocrotophos+SX, moxidectin+SX, naled+SX, nicofluprole+SX, nicotine+SX, nicotine-sulfate+SX, nitenpyram+SX, novaluron+SX, noviflumuron+SX, oil of the seeds of Chenopodium anthelminticum+SX, omethoate+SX, oxamyl+SX, oxazosulfyl+SX, oxydemeton-methyl+SX, parathion+SX, parathion-methyl+SX, permethrin+SX, phenothrin+SX, phenthoate+SX, phorate+SX, phosalone+SX, phosmet+SX, phosphamidon+SX, phosphine+SX, phoxim+SX, pirimicarb+SX, pirimiphos-methyl+SX, prallethrin+SX, profenofos+SX, profluthrin+SX, propargite+SX, propetamphos+SX, propoxur+SX, propylene glycol alginate+SX, prothiofos+SX, pyflubumide+SX, pymetrozine+SX, pyraclofos+SX, pyrethrins+SX, pyridaben+SX, pyridalyl+SX, pyridaphenthion+SX, pyrifluquinazone+SX, pyrimidifen+SX, pyriminostrobin+SX, pyriprole+SX, pyriproxyfen+SX, quinalphos+SX, resmethrin+SX, rotenone+SX, ryanodine+SX, sarolaner+SX, selamectin+SX, sigma-cypermethrin+SX, silafluofen+SX, sodium borate+SX, sodium metaborate+SX, spidoxamat+SX, spinetoram+SX, spinosad+SX, spirodiclofen+SX, spiromesifen+SX, spiropidion+SX, spirotetramat+SX, sulfluramid+SX, sulfotep+SX, sulfoxaflor+SX, sulfur+SX, sulfuryl fluoride+SX, tartar emetic+SX, tau-fluvalinate+SX, tebufenozide+SX, tebufenpyrad+SX, tebupirimfos+SX, teflubenzuron+SX, tefluthrin+SX, temephos+SX, terbufos+SX, terpene constituents of the extract of Chenopodium ambrosioides near ambrosioides+SX, tetrachlorantraniliprole+SX, tetrachlorvinphos+SX, tetradifon+SX, tetramethrin+SX, tetramethylfluthrin+SX, tetraniliprole+SX, theta-cypermethrin+SX, thiacloprid+SX, thiamethoxam+SX, thiocyclam+SX, thiodicarb+SX, thiofanox+SX, thiometon+SX, thiosultap-disodium+SX, thiosultap-monosodium+SX, tioxazafen+SX, tolfenpyrad+SX, tralomethrin+SX, transfluthrin+SX, triazamate+SX, triazophos+SX, trichlorfon+SX, triflumezopyrim+SX, triflumuron+SX, trimethacarb+SX, tyclopyrazoflor+SX, vamidothion+SX, wood extract of Quassia amara+SX, XMC (3,5-dimethylphenyl N-methylcarbamate)+SX, xylylcarb+SX, zeta-cypermethrin+SX, zinc phosphide+SX, 4-[5-(3,5-dichlorophenyl)-5-(trifluoromethyl)-4,5-dihydro-1,2-oxazol-3-yl]-2-methyl-N-(1-oxothietan-3-yl)benzamide (1241050-20-3)+SX, 3-methoxy-N-(5-{5-(trifluoromethyl)-5-[3-(trifluoromethyl)phenyl]-4,5-dihydro-1,2-oxazol-3-yl}indan-1-yl)propanamide (1118626-57-5)+SX, 2-({2-fluoro-4-methyl-5-[(2,2,2-trifluoroethyl)sulfinyl]phenyl}imino)-3-(2,2,2-trifluoroethyl)-1,3-thiazolidin-4-one (1445683-71-5)+SX, (2Z)-2-({2-fluoro-4-methyl-5-[(R)-(2,2,2-trifluoroethyl)sulfinyl]phenyl}imino)-3-(2,2,2-trifluoroethyl)-1,3-thiazolidin-4-one (2377084-09-6)+SX, (3R)-3-(2-chlorothiazol-5-yl)-8-methyl-7-oxo-6-phenyl-2,3-dihydrothiazolo[3,2-a]pyrimidin-4-ium-5-olate (2249718-27-0)+SX, N-{4-chloro-3-[(1-cyanocyclopropyl)carbamoyl]phenyl}-1-methyl-4-(methanesulfonyl)-3-(1,1,2,2,2-pentafluoroethyl)-1H-pyrazole-3-carboxamide (1400768-21-9)+SX, N-[3-chloro-1-(pyridin-3-yl)-1H-pyrazol-4-yl]-2-(methanesulfonyl)propanamide (2396747-83-2)+SX, 1,4-dimethyl-2-[2-(pyridin-3-yl)-2H-indazol-5-yl]-1,2,4-triazolidine-3,5-dione (2171099-09-3)+SX, 2-isopropyl-5-[(3,4,4-trifluoro-3-buten-1-yl)sulfonyl]-1,3,4-thiadiazole (2058052-95-0)+SX, N-({2-fluoro-4-[(2S,3S)-2-hydroxy-3-(3,4,5-trichlorophenyl)-3-(trifluoromethyl)pyrrolidin-1-yl]phenyl}methyl)cyclopropanecarboxamide+SX, 7-fluoro-N-[1-(methylsulfanyl)-2-methylpropan-2-yl]-2-(pyridin-3-yl)-2H-indazole-4-carboxamide+SX, 7-fluoro-N-[1-(methanesulfinyl)-2-methylpropan-2-yl]-2-(pyridin-3-yl)-2H-indazole-4-carboxamide+SX, 7-fluoro-N-[1-(methanesulfonyl)-2-methylpropan-2-yl]-2-(pyridin-3-yl)-2H-indazole-4-carboxamide+SX, N-(1-methylcyclopropyl)-2-(pyridin-3-yl)-2H-indazole-4-carboxamide+SX, N-[1-(difluoromethyl)cyclopropyl]-2-(pyridin-3-yl)-2H-indazole-4-carboxamide+SX, 2,9-dihydro-9-(methoxymethyl)-2-(pyridin-3-yl)-10H-pyrazolo[3,4-f]pyrido[2,3-b][1,4]oxazepin-10-one (2607927-97-7)+SX, BT crop protein Cry1Ab+SX, BT crop protein Cry1Ac+SX, BT crop protein Cry1Fa+SX, BT crop protein Cry1A.105+SX, BT crop protein Cry2Ab+SX, BT crop protein Vip3A+SX, BT crop protein mCry3A+SX, BT crop protein Cry3Ab+SX, BT crop protein Cry3Bb+SX, BT crop protein Cry34Ab1/Cry35Ab1+SX, Adoxophyes orana granulosis virus strain BV-0001+SX, Anticarsia gemmatalis mNPV+SX, Autographa californica mNPV+SX, Cydia pomonella GV strain V15+SX, Cydia pomonella GV strain V22+SX, Cryptophlebia leucotreta GV+SX, Dendrolimus punctatus cypovirus+SX, Helicoverpa armigera NPV strain BV-0003+SX, Helicoverpa zea NPV+SX, Lymantria dispar NPV+SX, Mamestra brassicae NPV+SX, Mamestra configurata NPV+SX, Neodiprion abietis NPV+SX, Neodiprion lecontei NPV+SX, Neodiprion sertifer NPV+SX, Nosema locustae+SX, Orgyia pseudotsugata NPV+SX, Pieris rapae GV+SX, Plodia interpunctella GV+SX, Spodoptera exigua mNPV+SX, Spodoptera littoralis mNPV+SX, Spodoptera litura NPV+SX, Arthrobotrys dactyloides+SX, Bacillus firmus strain GB-126+SX, Bacillus firmus strain I-1582+SX, Bacillus firmus strain NCIM2637+SX, Bacillus megaterium+SX, Bacillus sp. strain AQ175+SX, Bacillus sp. strain AQ177+SX, Bacillus sp. strain AQ178+SX, Bacillus sphaericus strain 2362 serotype H5a5b+SX, Bacillus sphaericus strain ABTS1743+SX, Bacillus sphaericus Serotype strain H5a5b+SX, Bacillus thuringiensis strain AQ52+SX, Bacillus thuringiensis strain BD #32+SX, Bacillus thuringiensis strain CR-371+SX, Bacillus thuringiensis subsp. Aizawai strain ABTS-1857+SX, Bacillus thuringiensis subsp. Aizawai strain AM65-52+SX, Bacillus thuringiensis subsp. Aizawai strain GC-91+SX, Bacillus thuringiensis subsp. Aizawai strain NB200+SX, Bacillus thuringiensis subsp. Aizawai Serotype strain H-7+SX, Bacillus thuringiensis subsp. Kurstaki strain ABTS351+SX, Bacillus thuringiensis subsp. Kurstaki strain BMP123+SX, Bacillus thuringiensis subsp. Kurstaki strain CCT1306+SX, Bacillus thuringiensis subsp. Kurstaki strain EG2348+SX, Bacillus thuringiensis subsp. Kurstaki strain EG7841+SX, Bacillus thuringiensis subsp. Kurstaki strain EVB113-19+SX, Bacillus thuringiensis subsp. Kurstaki strain F810+SX, Bacillus thuringiensis subsp. Kurstaki strain HD-1+SX, Bacillus thuringiensis subsp. Kurstaki strain PB54+SX, Bacillus thuringiensis subsp. Kurstaki strain SA-11+SX, Bacillus thuringiensis subsp. Kurstaki strain SA-12+SX, Bacillus thuringiensis subsp. Tenebriosis strain NB176+SX, Bacillus thuringiensis subsp. Thuringiensis strain MPPL002+SX, Bacillus thuringiensis subsp. morrisoni+SX, Bacillus thuringiensis var. colmeri+SX, Bacillus thuringiensis var. darmstadiensis strain 24-91+SX, Bacillus thuringiensis var. dendrolimus+SX, Bacillus thuringiensis var. galleriae+SX, Bacillus thuringiensis var. israelensis strain BMP144+SX, Bacillus thuringiensis var. israelensis serotype strain H-14+SX, Bacillus thuringiensis var. japonensis strain buibui+SX, Bacillus thuringiensis var. san diego strain M-7+SX, Bacillus thuringiensis var. 7216+SX, Bacillus thuringiensis var. aegypti+SX, Bacillus thuringiensis var. T36+SX, Beauveria bassiana strain ANT-03+SX, Beauveria bassiana strain ATCC74040+SX, Beauveria bassiana strain GHA+SX, Beauveria brongniartii+SX, Burkholderia rinojensis strain A396+SX, Chromobacterium subtsugae strain PRAA4-1T+SX, Dactyllela ellipsospora+SX, Dectylaria thaumasia+SX, Hirsutella minnesotensis+SX, Hirsutella rhossiliensis+SX, Hirsutella thompsonii+SX, Lagenidium giganteum+SX, Lecanicillium lecanii strain KV01+SX, Lecanicillium lecanii conidia of strain DAOM198499+SX, Lecanicillium lecanii conidia of strain DAOM216596+SX, Lecanicillium muscarium strain Ve6+SX, Metarhizium anisopliae strain F52+SX, Metarhizium anisopliae var. acridum+SX, Metarhizium anisopliae var. anisopliae BIPESCO 5/F52+SX, Metarhizium flavoviride+SX, Monacrosporium phymatopagum+SX, Paecilomyces fumosoroseus Apopka strain 97+SX, Paecilomyces lilacinus strain 251+SX, Paecilomyces tenuipes strain T1+SX, Paenibacillus popilliae+SX, Pasteuria nishizawae strain Pn1+SX, Pasteuria penetrans+SX, Pasteuria usgae+SX, Pasteuria thornei+SX, Serratia entomophila+SX, Verticillium chlamydosporium+SX, Verticillium lecani strain NCIM1312+SX, Wolbachia pipientis+SX.

Combinations of the Present ingredient in the above Group (b) and the Compound of the present invention:

acibenzolar-S-methyl+SX, aldimorph+SX, ametoctradin+SX, aminopyrifen+SX, amisulbrom+SX, anilazine+SX, azaconazole+SX, azoxystrobin+SX, basic copper sulfate+SX, benalaxyl+SX, benalaxyl-M+SX, benodanil+SX, benomyl+SX, benthiavalicarb+SX, benthiavalicarb-isopropyl+SX, benzovindiflupyr+SX, binapacryl+SX, biphenyl+SX, bitertanol+SX, bixafen+SX, blasticidin-S+SX, Bordeaux mixture+SX, boscalid SX, bromothalonil+SX, bromuconazole+SX, bupirimate+SX, captafol+SX, captan+SX, carbendazim+SX, carboxin+SX, carpropamid+SX, chinomethionat+SX, chitin+SX, chloroinconazide+SX, chloroneb+SX, chlorothalonil+SX, chlozolinate+SX, colletochlorin B+SX, copper(II) acetate+SX, copper(II) hydroxide+SX, copper oxychloride+SX, copper(II) sulfate+SX, coumoxystrobin+SX, cyazofamid+SX, cyflufenamid+SX, cymoxanil+SX, cyproconazole+SX, cyprodinil+SX, dichlobentiazox+SX, dichlofluanid+SX, diclocymet+SX, diclomezine+SX, dicloran+SX, diethofencarb+SX, difenoconazole+SX, diflumetorim+SX, dimethachlone+SX, dimethirimol+SX, dimethomorph+SX, dimoxystrobin+SX, diniconazole+SX, diniconazole-M+SX, dinocap+SX, dipotassium hydrogenphosphite+SX, dipymetitrone+SX, dithianon+SX, dodecylbenzenesulphonic acid bisethylenediamine copper(II) salt+SX, dodemorph+SX, dodine+SX, edifenphos+SX, enoxastrobin+SX, epoxiconazole+SX, etaconazole+SX, ethaboxam+SX, ethirimol+SX, etridiazole+SX, extract of Melaleuca alternifolia+SX, extract of Reynoutria sachalinensis+SX, extract of the cotyledons of lupine plantlets (“BLAD”)+SX, extract of Allium sativum+SX, extract of Equisetum arvense+SX, extract of Tropaeolum majus+SX, famoxadone+SX, fenamidone+SX, fenaminstrobin+SX, fenarimol+SX, fenbuconazole+SX, fenfuram+SX, fenhexamid+SX, fenoxanil+SX, fenpiclonil+SX, fenpicoxamid+SX, fenpropidin+SX, fenpropimorph+SX, fenpyrazamine+SX, fentin acetate+SX, fentin chloride+SX, fentin hydroxide+SX, ferbam+SX, ferimzone+SX, florylpicoxamid+SX, fluazinam+SX, flubeneteram+SX, fludioxonil+SX, flufenoxadiazam+SX, flufenoxystrobin+SX, fluindapyr+SX, flumetylsulforim+SX, flumorph+SX, fluopicolide+SX, fluopyram+SX, fluopimomide+SX, fluoroimide+SX, fluoxapiprolin+SX, fluoxastrobin+SX, fluoxytioconazole+SX, fluquinconazole+SX, flusilazole+SX, flusulfamide+SX, flutianil+SX, flutolanil+SX, flutriafol+SX, fluxapyroxad+SX, folpet+SX, fosetyl+SX, fosetyl-aluminium+SX, fuberidazole+SX, furalaxyl+SX, furametpyr+SX, guazatine+SX, hexaconazole+SX, hymexazole+SX, imazalil+SX, imibenconazole+SX, iminoctadine+SX, iminoctadine triacetate+SX, inpyrfluxam+SX, iodocarb+SX, ipconazole+SX, ipfentrifluconazole+SX, ipflufenoquin+SX, iprobenfos+SX, iprodione+SX, iprovalicarb+SX, isofetamid+SX, isoflucypram+SX, isoprothiolane+SX, isopyrazam+SX, isotianil+SX, kasugamycin+SX, kresoxim-methyl+SX, laminarin+SX, leaves and bark of Quercus+SX, mancozeb+SX, mandestrobin+SX, mandipropamid+SX, maneb+SX, mefentrifluconazole+SX, mepanipyrim+SX, mepronil+SX, meptyldinocap+SX, metalaxyl+SX, metalaxyl-M+SX, metarylpicoxamid+SX, metconazole+SX, methasulfocarb+SX, metiram+SX, metominostrobin+SX, metrafenone+SX, metyltetraprole+SX, myclobutanil+SX, naftifine+SX, nuarimol+SX, octhilinone+SX, ofurace+SX, orysastrobin+SX, oxadixyl+SX, oxathiapiprolin+SX, oxine-copper+SX, oxolinic acid+SX, oxpoconazole+SX, oxpoconazole fumarate+SX, oxycarboxin+SX, oxytetracycline+SX, pefurazoate+SX, penconazole+SX, pencycuron+SX, penflufen+SX, penthiopyrad+SX, phenamacril+SX, phosphorous acid+SX, phthalide+SX, picarbutrazox+SX, picoxystrobin+SX, piperalin+SX, polyoxins+SX, potassium hydrogencarbonate+SX, potassium dihydrogenphosphite+SX, probenazole+SX, prochloraz+SX, procymidone+SX, propamidine+SX, propamocarb+SX, propiconazole+SX, propineb+SX, proquinazid+SX, prothiocarb+SX, prothioconazole+SX, pydiflumetofen+SX, pyraclostrobin+SX, pyrametostrobin+SX, pyraoxystrobin+SX, pyrapropoyne+SX, pyraziflumid+SX, pyrazophos+SX, pyribencarb+SX, pyributicarb+SX, pyridachlometyl+SX, pyrifenox+SX, pyrimethanil+SX, pyrimorph+SX, pyriofenone+SX, pyrisoxazole+SX, pyroquilon+SX, Quillaja extract+SX, quinconazole+SX, quinofumelin+SX, quinoxyfen+SX, quintozene+SX, Saponins of Chenopodium quinoa+SX, seboctylamine+SX, sedaxane+SX, silthiofam+SX, simeconazole+SX, sodium hydrogencarbonate+SX, spiroxamine+SX, streptomycin+SX, sulfur+SX, tebuconazole+SX, tebufloquin+SX, teclofthalam+SX, tecnazene+SX, terbinafine+SX, tetraconazole+SX, thiabendazole+SX, thifluzamide+SX, thiophanate+SX, thiophanate-methyl+SX, thiram+SX, thymol+SX, tiadinil+SX, tolclofos-methyl+SX, tolfenpyrad+SX, tolprocarb+SX, tolylfluanid+SX, triadimefon+SX, triadimenol+SX, triazoxide+SX, triclopyricarb+SX, tricyclazole+SX, tridemorph+SX, trifloxystrobin+SX, triflumizole+SX, triforine+SX, triticonazole+SX, validamycin+SX, valifenalate+SX, vinclozolin+SX, yellow mustard powder+SX, zinc thiazole+SX, zineb+SX, ziram+SX, zoxamide+SX, N′-[4-({3-[(4-chlorophenyl)methyl]-1,2,4-thiadiazol-5-yl}oxy)-2,5-dimethylphenyl]-N-ethyl-N-methylmethanimidamide (1202781-91-6)+SX, N′-{4-[(4,5-dichlorothiazol-2-yl)oxy]-2,5-dimethylphenyl}-N-ethyl-N-methylmethanimidamide (929908-57-6)+SX, N′-(2,5-dimethyl-4-phenoxyphenyl)-N-ethyl-N-methylmethanimidamide (1052688-31-9)+SX, N′-[5-chloro-4-(2-fluorophenoxy)-2-methylphenyl]-N-ethyl-N-methylmethanimidamide (2055589-28-9)+SX, N′-[2-chloro-4-(2-fluorophenoxy)-5-methylphenyl]-N-ethyl-N-methylmethanimidamide (2055756-21-1)+SX, N′-(2-chloro-4-phenoxy-5-methylphenyl)-N-ethyl-N-methylmethanimidamide (2062599-39-5)+SX, N′-[4-(1-hydroxy-1-phenyl-2,2,2-trifluoroethyl)-2-methyl-5-methoxyphenyl]-N-isopropyl-N-methylmethanimidamide (2101814-55-3)+SX, N′-[5-bromo-6-(1-methyl-2-propoxyethoxy)-2-methylpyridin-3-yl]-N-ethyl-N-methylmethanimidamide (1817828-69-5)+SX, 4-(2-bromo-4-fluorophenyl)-N-(2-chloro-6-fluorophenyl)-1,3-dimethyl-1H-pyrazol-5-amine (1362477-26-6)+SX, 2-[6-(3-fluoro-4-methoxyphenyl)-5-methylpyridin-2-yl]quinazoline (1257056-97-5)+SX, ethyl (2Z)-3-amino-2-cyano-3-phenylacrylate (39491-78-6)+SX, N-[(2-chlorothiazol-5-yl)methyl]-N-ethyl-6-methoxy-3-nitropyridin-2-amine (1446247-98-8)+SX, 5-(4-chlorobenzyl)-2-(chloromethyl)-2-methyl-1-(1H-1,2,4-triazol-1-ylmethyl)cyclopentan-1-ol (1394057-11-4)+SX, (1R, 2S, 5S)-5-(4-chlorobenzyl)-2-(chloromethyl)-2-methyl-1-(1H-1,2,4-triazol-1-ylmethyl)cyclopentan-1-ol (1801930-06-2)+SX, (1S, 2R, 5R)-5-(4-chlorobenzyl) (chloromethyl)-2-methyl-1-(1H-1,2,4-triazol ylmethyl)cyclopentan-1-ol (1801930-07-3)+SX, 2-(chloromethyl)-5-(4-fluorobenzyl)-2-methyl-1-(1H-1,2,4-triazol-1-ylmethyl)cyclopentan-1-ol (1394057-13-6)+SX, (1R, 2S, 5S)-2-(chloromethyl)-5-(4-fluorobenzyl)-2-methyl-1-(1H-1,2,4-triazol-1-ylmethyl)cyclopentan-1-ol (1801930-08-4)+SX, (1S, 2R, 5R)-2-(chloromethyl)-5-(4-fluorobenzyl)-2-methyl-1-(1H-1,2,4-triazol-1-ylmethyl)cyclopentan-1-ol (1801930-09-5)+SX, methyl 3-[(4-chlorophenyl)methyl]-2-hydroxy-1-methyl-2-(1H-1,2,4-triazol-1-ylmethyl)cyclopentan-1-carboxylate (1791398-02-1)+SX, 1-(2,4-difluorophenyl)-2-(1H-1,2,4-triazol-1-yl)-1-[1-(4-bromo-2,6-difluorophenoxy)cyclopropyl]ethanol (2019215-86-0)+SX, 1-(2,4-difluorophenyl)-2-(1H-1,2,4-triazol-1-yl)-1-[1-(4-chloro-2,6-difluorophenoxy)cyclopropyl]ethanol (2019215-84-8)+SX, 1-[2-(1-chlorocyclopropyl)-3-(2-fluorophenyl)-2-hydroxypropyl]-1H-imidazole-5-carbonitrile (2018316-13-5)+SX, 1-[2-(1-chlorocyclopropyl)-3-(2,3-difluorophenyl)-2-hydroxypropyl]-1H-imidazole-5-carbonitrile (2018317-25-2)+SX, 2-[6-(4-bromophenoxy)-2-(trifluoromethyl)pyridin-3-yl]-1-(1H-1,2,4-triazol-1-yl)propan-2-ol (2082661-43-4)+SX, 2-[6-(4-chlorophenoxy)-2-(trifluoromethyl)pyridin-3-yl]-1-(1H-1,2,4-triazol-1-yl)propan-2-ol (2082660-27-1)+SX, methyl ({2-methyl-5-[1-(4-methoxy-2-methylphenyl)-1H-pyrazol-3-yl]phenyl}methyl)carbamate (1605879-98-8)+SX, 2-(difluoromethyl)-N-[1,1,3-trimethyl-2,3-dihydro-1H-inden-4-yl]pyridine-3-carboxamide (1616239-21-4)+SX, 2-(difluoromethyl)-N-[3-ethyl-1,1-dimethyl-2,3-dihydro-1H-inden-4-yl]pyridine-3-carboxamide (1847460-02-9)+SX, 2-(difluoromethyl)-N-[3-propyl-1,1-dimethyl-2,3-dihydro-1H-inden-4-yl]pyridine-3-carboxamide (1847460-05-2)+SX, (2E,3Z)-5-{[1-(4-chlorophenyl)-1H-pyrazol-3-yl]oxy}-2-(methoxyimino)-N,3-dimethylpent-3-enamide (1445331-27-0)+SX, (2E,3Z)-5-{[1-(2,4-dichlorophenyl)-1H-pyrazol-3-yl]oxy}-2-(methoxyimino)-N,3-dimethylpent-3-enamide (1445331-54-3)+SX, 5-chloro-4-({2-[6-(4-chlorophenoxy)pyridin-3-yl]ethyl}amino)-6-methylpyrimidine (1605340-92-8)+SX, N-(1-benzyl-1,3-dimethylbutyl)-8-fluoroquinoline-3-carboxamide (2132414-04-9)+SX, N-(1-benzyl-3,3,3-trifluoro-1-methylpropyl)-8-fluoroquinoline-3-carboxamide (2132414-00-5)+SX, 4,4-dimethyl-2-({4-[5-(trifluoromethyl)-1,2,4-oxadiazol-3-yl]phenyl}methyl)isoxazolidin-3-one (2098918-25-1)+SX, 5,5-dimethyl-2-({4-[5-(trifluoromethyl)-1,2,4-oxadiazol-3-yl]phenyl}methyl)isoxazolidin-3-one (2098918-26-2)+SX, N-ethyl-2-methyl-N-({4-[5-(trifluoromethyl)-1,2,4-oxadiazol-3-yl]phenyl}methyl)propanamide+SX, N,2-dimethoxy-N-({4-[5-(trifluoromethyl)-1,2,4-oxadiazol yl]phenyl}methyl)propanamide+SX, N-methoxy-N-({4-[5-(trifluoromethyl)-1,2,4-oxadiazol yl]phenyl}methyl)cyclopropanecarboxamide+SX, N-methoxy-N′-methyl-N-({4-[5-(trifluoromethyl)-1,2,4-oxadiazol-3-yl]phenyl}methyl)urea+SX, N′-ethyl-N-methoxy-N-({4-[5-(trifluoromethyl)-1,2,4-oxadiazol-3-yl]phenyl}methyl)urea+SX, N,N′-dimethoxy-N-({4-[5-(trifluoromethyl)-1,2,4-oxadiazol-3-yl]phenyl}methyl)urea+SX, N-acetyl-2-(ethanesulfonyl)-N-[2-(methoxycarbonyl)-4-(trifluoromethoxy)phenyl]-4-(trifluoromethyl)benzamide (2043675-28-9)+SX, 3-(4-bromo-7-fluoroindol-1-yl)butan-2-yl N-[(3-hydroxy-4-methoxypyridin-2-yl)carbonyl]-L-alaninate+SX, 3-(7-bromoindol-1-yl)butan-2-yl N-[(3-hydroxy-4-methoxypyridin-2-yl)carbonyl]-L-alaninate+SX, 3-(7-bromo-4-fluoroindol-1-yl)butan-2-yl N-[(3-hydroxy-4-methoxypyridin-2-yl)carbonyl]-L-alaninate+SX, 3-(3,5-dichloropyridin-2-yl)butan-2-yl N-[(3-hydroxy-4-methoxypyridin-2-yl)carbonyl]-L-alaninate+SX, 3-(3,5-dichloropyridin-2-yl)butan-2-yl N-{[3-(acetoxymethoxy)-4-methoxypyridin-2-yl]carbonyl}-L-alaninate+SX, (1S)-1-[1-(naphthalen-1-yl)cyclopropyl]ethyl N-[(3-hydroxy-4-methoxypyridin-2-yl)carbonyl]-L-alaninate+SX, (1S)-1-[1-(naphthalen-1-yl)cyclopropyl]ethyl N-[(3-acetoxy-4-methoxypyridin-2-yl)carbonyl]-L-alaninate+SX, (1S)-1-[1-(naphthalen-1-yl)cyclopropyl]ethyl N-{[3-(acetoxymethoxy)-4-methoxypyridin-2-yl]carbonyl}-L-alaninate+SX, Agrobacterium radiobactor strain K1026+SX, Agrobacterium radiobactor strain K84+SX, Bacillus amyloliquefaciens strain PTA-4838 (Aveo (trademark) EZ Nematicide)+SX, Bacillus amyloliquefaciens strain AT332+SX, Bacillus amyloliquefaciens strain B3+SX, Bacillus amyloliquefaciens strain D747+SX, Bacillus amyloliquefaciens strain DB101+SX, Bacillus amyloliquefaciens strain DB102+SX, Bacillus amyloliquefaciens strain GB03+SX, Bacillus amyloliquefaciens strain FZB24+SX, Bacillus amyloliquefaciens strain FZB42+SX, Bacillus amyloliquefaciens strain IN937a+SX, Bacillus amyloliquefaciens strain MBI600+SX, Bacillus amyloliquefaciens strain QST713+SX, Bacillus amyloliquefaciens isolate strain B246+SX, Bacillus amyloliquefaciens strain F727+SX, Bacillus amyloliquefaciens subsp. plantarum strain D747+SX, Bacillus licheniformis strain HB-2+SX, Bacillus licheniformis strain SB3086+SX, Bacillus pumilus strain AQ717+SX, Bacillus pumilus strain BUF-33+SX, Bacillus pumilus strain GB34+SX, Bacillus pumilus strain QST2808+SX, Bacillus simplex strain CGF2856+SX, Bacillus subtilis strain AQ153+SX, Bacillus subtilis strain AQ743+SX, Bacillus subtilis strain BU1814+SX, Bacillus subtilis strain D747+SX, Bacillus subtilis strain DB101+SX, Bacillus subtilis strain FZB24+SX, Bacillus subtilis strain GB03+SX, Bacillus subtilis strain HAI0404+SX, Bacillus subtilis strain IAB/BS03+SX, Bacillus subtilis strain MBI600+SX, Bacillus subtilis strain QST30002/AQ30002+SX, Bacillus subtilis strain QST30004/AQ30004+SX, Bacillus subtilis strain QST713+SX, Bacillus subtilis strain QST714+SX, Bacillus subtilis var. Amyloliquefaciens strain FZB24+SX, Bacillus subtilis strain Y1336+SX, Burkholderia cepacia+SX, Burkholderia cepacia type Wisconsin strain J82+SX, Burkholderia cepacia type Wisconsin strain M54+SX, Candida oleophila strain 0+SX, Candida saitoana+SX, Chaetomium cupreum+SX, Clonostachys rosea+SX, Coniothyrium minitans strain CGMCC8325+SX, Coniothyrium minitans strain CON/M/91-8+SX, Cryptococcus albidus+SX, Erwinia carotovora subsp. carotovora strain CGE234M403+SX, Fusarium oxysporum strain Fo47+SX, Gliocladium catenulatum strain J1446+SX, Paenibacillus polymyxa strain AC-1+SX, Paenibacillus polymyxa strain BS-0105+SX, Pantoea agglomerans strain E325+SX, Phlebiopsis gigantea strain VRA1992+SX, Pseudomonas aureofaciens strain TX-1+SX, Pseudomonas chlororaphis strain 63-28+SX, Pseudomonas chlororaphis strain AFS009+SX, Pseudomonas chlororaphis strain MA342+SX, Pseudomonas fluorescens strain 1629RS+SX, Pseudomonas fluorescens strain A506+SX, Pseudomonas fluorescens strain CL145A+SX, Pseudomonas fluorescens strain G7090+SX, Pseudomonas sp. strain CAB-02+SX, Pseudomonas syringae strain 742RS+SX, Pseudomonas syringae strain MA-4+SX, Pseudozyma flocculosa strain PF-A22UL+SX, Pseudomonas rhodesiae strain HAI-0804+SX, Pythium oligandrum strain DV74+SX, Pythium oligandrum strain M1+SX, Streptomyces griseoviridis strain K61+SX, Streptomyces lydicus strain WYCD108US+SX, Streptomyces lydicus strain WYEC108+SX, Talaromyces flavus strain SAY-Y-94-01+SX, Talaromyces flavus strain V117b+SX, Trichoderma asperellum strain ICC012+SX, Trichoderma asperellum SKT-1+SX, Trichoderma asperellum strain T25+SX, Trichoderma asperellum strain T34+SX, Trichoderma asperellum strain TV1+SX, Trichoderma atroviride strain CNCM 1-1237+SX, Trichoderma atroviride strain LC52+SX, Trichoderma atroviride strain IMI 206040+SX, Trichoderma atroviride strain SC1+SX, Trichoderma atroviride strain SKT-1+SX, Trichoderma atroviride strain T11+SX, Trichoderma gamsii strain ICC080+SX, Trichoderma harzianum strain 21+SX, Trichoderma harzianum strain DB104+SX, Trichoderma harzianum strain DSM 14944+SX, Trichoderma harzianum strain ESALQ-1303+SX, Trichoderma harzianum strain ESALQ-1306+SX, Trichoderma harzianum strain IIHR-Th-2+SX, Trichoderma harzianum strain ITEM908+SX, Trichoderma harzianum strain kd+SX, Trichoderma harzianum strain MO1+SX, Trichoderma harzianum strain SF+SX, Trichoderma harzianum strain T22+SX, Trichoderma harzianum strain T39+SX, Trichoderma harzianum strain T78+SX, Trichoderma harzianum strain TH35+SX, Trichoderma polysporum strain IMI206039+SX, Trichoderma stromaticum+SX, Trichoderma virens strain G-41+SX, Trichoderma virens strain GL-21+SX, Trichoderma viride+SX, Variovorax paradoxus strain CGF4526+SX, Harpin protein+SX.

Combinations of the Present ingredient in the above Group (c) and the Compound of the present invention:

1-methylcyclopropene+SX, 1,3-diphenylurea+SX, 2,3,5-triiodobenzoic acid+SX, IAA ((1H-indol-3-yl)acetic acid)+SX, IBA (4-(1H-indol-3-yl)butyric acid)+SX, MCPA (2-(4-chloro-2-methylphenoxy)acetic acid)+SX, MCPB (4-(4-chloro-2-methylphenoxy)butyric acid)+SX, 4-CPA (4-chlorophenoxyacetic acid)+SX, 5-aminolevulinic acid hydrochloride+SX, 6-benzylaminopurine+SX, abscisic acid+SX, AVG (aminoethoxyvinylglycine)+SX, anisiflupurin+SX, ancymidol+SX, butralin+SX, calcium carbonate+SX, calcium chloride+SX, calcium formate+SX, calcium peroxide+SX, calcium polysulfide+SX, calcium sulfate+SX, chlormequat-chloride+SX, chlorpropham+SX, choline chloride+SX, cloprop+SX, cyanamide+SX, cyclanilide+SX, daminozide+SX, decan-1-ol+SX, dichlorprop+SX, dikegulac+SX, dimethipin+SX, diquat+SX, ethephon+SX, ethychlozate+SX, flumetralin+SX, flurprimidol+SX, forchlorfenuron+SX, formononetin+SX, Gibberellin A+SX, Gibberellin A3+SX, inabenfide+SX, Kinetin+SX, lipochitooligosaccharide SP104+SX, maleic hydrazide+SX, mefluidide+SX, mepiquat-chloride+SX, oxidized glutathione+SX, paclobutrazol+SX, pendimethalin+SX, prohexadione-calcium+SX, prohydrojasmon+SX, pyraflufen-ethyl+SX, sintofen+SX, sodium 1-naphthaleneacetate+SX, sodium cyanate+SX, thidiazuron+SX, triapenthenol+SX, tribufos+SX, trinexapac-ethyl+SX, uniconazole-P+SX, 2-(naphthalen-1-yl)acetamide+SX, [4-oxo-4-(2-phenylethyl)amino]butyric acid+SX, methyl 5-(trifluoromethyl)benzo[b]thiophene-2-carboxylate+SX, 3-[(6-chloro-4-phenylquinazolin-2-yl)amino]propan-1-ol+SX, Claroideoglomus etunicatum+SX, Claroideoglomus claroideum+SX, Funneliformis mosseae+SX, Gigaspora margarita+SX, Gigaspora rosea+SX, Glomus aggregatum+SX, Glomus deserticola+SX, Glomus monosporum+SX, Paraglomus brasillianum+SX, Rhizophagus clarus+SX, Rhizophagus intraradices RTI-801+SX, Rhizophagus irregularis DAOM 197198+SX, Azorhizobium caulinodans+SX, Azospirillum amazonense+SX, Azospirillum brasilense XOH+SX, Azospirillum brasilense Ab-V5+SX, Azospirillum brasilense Ab-V6+SX, Azospirillum caulinodans+SX, Azospirillum halopraeferens+SX, Azospirillum irakense+SX, Azospirillum lipoferum+SX, Bradyrhizobium elkanii SEMIA 587+SX, Bradyrhizobium elkanii SEMIA 5019+SX, Bradyrhizobium japonicum TA-11+SX, Bradyrhizobium japonicum USDA 110+SX, Bradyrhizobium liaoningense+SX, Bradyrhizobium lupini+SX, Delftia acidovorans RAY209+SX, Mesorhizobium ciceri+SX, Mesorhizobium huakii+SX, Mesorhizobium loti+SX, Rhizobium etli+SX, Rhizobium galegae+SX, Rhizobium leguminosarum bv. Phaseoli+SX, Rhizobium leguminosarum bv. Trifolii+SX, Rhizobium leguminosarum bv. Viciae+SX, Rhizobium trifolii+SX, Rhizobium tropici+SX, Sinorhizobium fredii+SX, Sinorhizobium meliloti+SX, Zucchini Yellow Mosaik Virus weak strain+SX.

Combinations of the Present ingredient in the above Group (d) and the Compound of the present invention:

anthraquinone+SX, deet+SX, icaridin+SX.

Examples of the ratio of the Compound of the present invention and the Present ingredient include, but are not limited to 1000:1 to 1:1000, 500:1 to 1:500, 100:1 to 1:100, 50:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, and 1:50, in the ratio by weight (Compound of the present invention:Present ingredient).

The Present compound, the Compound of the present invention, or the Composition A is usually used by mixing it with inert carrier(s) such as solid carrier(s), liquid carrier(s), and gaseous carrier(s), surfactant(s), and the like, and as needed, adding thereto auxiliary agent(s) for formulation such as binder(s), dispersant(s), and stabilizer(s) to be formulated into an aqueous suspension formulation, an oily suspension formulation, an oil solution, an emulsifiable concentrate, an emulsion formulation, a microemulsion formulation, a microcapsule formulation, a wettable powder, a granular wettable powder, a dust formulation, a granule, a tablet, an aerosol formulation, a resin formulation, or the like. In addition to these formulations, the Present compound, the Compound of the present invention, or the Composition A may be used by formulating it into a dosage form described in Manual on development and use of FAO and WHO Specifications for pesticides, FAO Plant Production and Protection Papers-271-276, prepared by the FAO/WHO Joint Meeting on Pesticide Specifications, 2016, ISSN: 0259-2517.

These formulations usually comprise 0.0001 to 99% by weight ratio of the Present compound, the Compound of the present invention, or the Composition A.

Examples of the solid carrier include fine powders and granules of clays (for example, pyrophyllite clay and kaolin clay), talc, calcium carbonate, diatomaceous earth, zeolite, bentonite, acid white clay, attapulgite, white carbon, ammonium sulfate, vermiculite, perlite, pumice, silica sand, chemical fertilizers (for example, ammonium sulfate, ammonium phosphate, ammonium nitrate, urea, and ammonium chloride), and the others; as well as resins (for example, polypropylene, polyester, polyurethane, polyamide, and polyvinyl chloride).

Examples of the liquid carrier include water, alcohols (for example, ethanol, cyclohexanol, benzyl alcohol, propylene glycol, and polyethylene glycol), ketones (for example, acetone and cyclohexanone), aromatic hydrocarbons (for example, xylene, phenyl xylyl ethane, and methylnaphthalene), aliphatic hydrocarbons (for example, hexane and cyclohexane), esters (for example, ethyl acetate, methyl oleate, and propylene carbonate), nitriles (for example, acetonitrile), ethers (for example, ethylene glycol dimethyl ether), amides (for example, N,N-dimethylformamide and N,N-dimethyloctanamide), sulfoxides (for example, dimethylsulfoxide), lactams (for example, N-methylpyrrolidone and N-octylpyrrolidone), fatty acids (for example, oleic acid), and vegetable oils (for example, soybean oil).

Examples of the gaseous carrier include fluorocarbon, butane gas, LPG (liquefied petroleum gas), dimethyl ether, nitrogen, and carbon dioxide.

Examples of the surfactant include nonionic surfactants (for example, polyoxyethylene alkyl ethers, polyoxyethylene alkyl aryl ethers, and polyethylene glycol fatty acid esters), and anionic surfactants (for example, alkyl sulfonates, alkyl aryl sulfonates, and alkyl sulfates). Specific examples thereof include Nimbus (registered trademark), Assist (registered trademark), Aureo (registered trademark), Iharol (registered trademark), Silwet L-77 (registered trademark), BreakThru (registered trademark), Sundancell (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), and BANOLE (registered trademark).

Examples of the other auxiliary agent for formulation include binders, dispersants, colorants, and stabilizers, and the specific examples thereof include polysaccharides (for example, starch, gum arabic, cellulose derivatives, and alginic acid), lignin derivatives, water-soluble synthetic polymers (for example, polyvinyl alcohol, polyvinyl pyrrolidone, and polyacrylic acids), acidic isopropyl phosphate, and dibutylhydroxytoluene.

Examples of the method for applying the Present compound, the Compound of the present invention, or the Composition A include a method for spraying it to soybean foliage, a method for treating it to seeds, and a method for applying it to soil for cultivating soybeans.

The amount of the Present compound, the Compound of the present invention, or the Composition A to be applied may be varied depending on the climate condition, dosage form, application period, application method, application site, diseases to be controlled, crops to be protected, and the like. In case of spray to soybean foliage or application to soil for cultivating soybeans, the amount of the Present compound, the Compound of the present invention, or the Composition A is usually within the range of 1 to 500 g, preferably 2 to 200 g, per 1000 m². In case of treatment to seeds, the amount of the Present compound, the Compound of the present invention, or the Composition A to be applied is usually within the range of 0.001 to 100 g, preferably 0.01 to 50 g, per 1 Kg of seeds. An emulsifiable concentrate, a wettable powder, a suspension, and the like are usually diluted with water and then applied. In this case, the concentration of the Present compound, the Compound of the present invention, or the Composition A after dilution is usually within the range of 0.0005 to 2% by weight, preferably 0.005 to 2% by weight. A dust formulation, a granule, and the like are usually applied as themselves without diluting them.

Examples of the seed treatment include application of the Compound of the present invention or the Composition A to seeds. Specific examples thereof include spray treatment wherein mist of a suspension of the Compound of the present invention or the Composition A is sprayed to seed surfaces; smear treatment wherein the Compound of the present invention or the Composition A is smeared to seeds; immersion treatment wherein seeds are immersed in a drug solution of the Compound of the present invention or the Composition A for a period of time; and methods for coating seeds by a carrier comprising the Compound of the present invention or the Composition A (for example, film coat treatment and pellet coat treatment).

When the Composition A is applied to seeds, a formulation of the Composition A may be applied to seeds, or a plurality of different formulations of the Composition A may be applied separately in a plurality of times to seeds. Examples of the method for applying a plurality of different formulations of the Composition A separately in a plurality of times include a method wherein a formulation comprising the Compound of the present invention only as an active ingredient is applied to seeds, said seeds are air-dried, and a formulation comprising the Present ingredient(s) is applied thereto; and a method wherein a formulation comprising the Compound of the present invention and the Present ingredient(s) as active ingredients is applied to seeds, said seeds are air-dried, and then a formulation comprising the Present ingredient(s) other than the applied Present ingredient(s) is applied.

In the present invention, the seed holding the Compound of the present invention or the Composition A means a seed in which the Compound of the present invention or the Composition A is attached to the surface of the seed. A material other than the Compound of the present invention or the Composition A may be attached to the above seed holding the Compound of the present invention or the Composition A before or after the Compound of the present invention or the Composition A is attached to the seed.

Also, when the Composition A is attached to surfaces of seeds to form layer(s), said layer(s) consist(s) of a layer or a plurality of layers. When said layer(s) consist(s) of a plurality of layers, each layer consists of a layer comprising one or more active ingredient(s), or consists of a layer comprising one or more active ingredient(s) and a layer comprising no active ingredient.

The seeds holding the Compound of the present invention or the Composition A may be prepared by, for example, applying a formulation comprising the Compound of the present invention or the Composition A to seeds by the above seed treatment method.

The above soybeans may be soybeans producible by natural crossing, soybeans producible by a mutation, F1 hybrid soybeans, and transgenic soybeans (also referred to as “genetically modified soybeans”). These soybeans generally have characteristics such as resistance to herbicides, accumulation of substances harmful to pests (also referred to as “resistance to pests”), sensitivity suppression against diseases (also referred to as “resistance to diseases”), increase in yield potential, improvement in resistance to biotic and abiotic stress factors, and quality modification of products (for example, increase and decrease in component contents, change in composition, and improvement in preservability or processability). Examples of technique for producing the above soybeans include conventional breeding techniques; gene-recombination techniques; genome breeding techniques; new breeding techniques; and genome-editing techniques.

Examples of soybeans having resistance to herbicides (soybeans resistant to herbicides) include soybeans resistant to auxin type herbicides such as 2,4-D and dicamba; soybeans resistant to glufosinate, soybeans resistant to glyphosate, soybeans resistant to isoxaflutole, soybeans resistant to 4-hydroxyphenylpyruvate dioxygenase inhibiting herbicides such as mesotrione; soybeans resistant to acetolactate synthase (ALS) inhibiting herbicides such as imidazolinone herbicides and sulfonylurea herbicides; and soybeans resistant to protoporphyrinogen oxidase inhibiting herbicides such as flumioxazin.

Soybeans that have acquired resistance to herbicides by gene-recombination techniques may be obtained by introducing a foreign gene (for example, gene of another organism such as a microorganism). For example, resistance to 2,4-D may be obtained by introducing a gene “aad-12” derived from Delftia acidovorans; resistance to dicamb may be obtained by introducing a gene “dmo” derived from Stenotrophomonas maltophilia strain DI-6; resistance to glufosinate may be obtained by introducing a gene “bar” derived from Streptomyces hygroscopicus or a gene “pat” derived from Streptomyces viridochromogenes; resistance to glyphosate may be obtained by introducing a gene “2mepsps” derived from Zea mays, a gene “CP4 epsps” derived from Agrobacterium tumefaciens strain CP4, or a gene “gat4601” derived from Bacillus licheniformis; resistance to isoxaflutole may be obtained by introducing a gene “hppdPF W336” derived from Pseudomonas fluorescens strain A32; resistance to mesotrione may be obtained by introducing a gene “avhppd-03” derived from Oat (Avena sativa); resistance to imidazolinone herbicides may be obtained by introducing a gene “csr1-2” derived from Arabidopsis thaliana; and resistance to sulfonylurea herbicides may be obtained by introducing a gene “gm-hra” derived from Glycine max.

Examples of soybeans that have acquired resistance to herbicides by a conventional breeding technique or a genome breeding technique include a soybean “STS (registered trademark) soybean” having resistance to sulfonylurea ALS inhibiting herbicides such as thifensulfuron-methyl.

Examples of soybeans that have acquired resistance to herbicides by a new breeding technique include a soybean in which a Roundup Ready (registered trademark) soybean having resistance to glyphosate is used as a rootstock, thereby resistance to glyphosate have been added to a nontransgenic soybeans graft (see Weed Technology, 2013, 27, 412.).

Examples of soybeans having resistance to pests include soybeans having resistance to Lepidoptera pests (for example, Pseudoplusia includens, Helicoverpa zea, and Spodoptera frugiperda), soybeans having resistance to Hemiptera pests (for example, Aphis glycines), and soybeans having resistance to Nematoda (for example, Heterodera glycines and Meloidogyne incognita).

Soybeans that have acquired resistance to pests by a gene-recombination technique may be obtained by introducing a foreign gene (for example, a gene encoding an insecticidal protein 5-endotoxin derived from Bacillus thuringiensis). For example, resistance to Lepidoptera pests may be obtained by introducing a gene “crylAc” derived from Bacillus thuringiensis subsp. Kurstaki strain HD73, a gene “cry1F” derived from Bacillus thuringiensis var. aizawai, a gene “cry1A.105” derived from Bacillus thuringiensis subsp. kumamotoensis, or a gene “cry2Ab2” derived from Bacillus thuringiensis subsp. kumamotoensis.

Examples of soybeans that have acquired resistance to pests by a conventional breeding technique or a genome breeding technique include a soybean having a gene showing resistance to soybean aphid, i.e., a “Rag1 (Resistance to Aphis glycines 1)” or a “Rag2 (Resistance to Aphis glycines 2)” gene, thereby showing resistance to soybean aphid (Aphis glycines) (see J. Econ. Entomol., 2015, 108, 326.); a soybean showing resistance to soybean cyst nematode (Heterodera glycines) (see Phytopathology, 2016, 106, 1444.); and a soybean “Fukuminori” showing resistance to cotton worm (Spodoptera litura).

Examples of soybeans that have acquired resistance to diseases include cultivars that have acquired resistance to soybean rust by a conventional breeding techniques or a gene-recombination technique. Examples of frequently used resistant gene include, but are not limited to, Rpp1, Rpp2, Rpp3, Rpp4, Rpp5, and Rpp6. Any one of these genes may be inserted into a soybean, or a combination of two or more of them may be inserted into a soybean. These genes are described in the following academic literatures and the like. Crop Science, 2007, 47, 837.; Theoretical and Applied Genetics, 2008, 117, 57.; Theoretical and Applied Genetics, 117, 545.; Crop Science, 2009, 49, 783.; Theoretical and Applied Genetics, 2009, 119, 271.; Theoretical and Applied Genetics, 2010, 121, 1023.; Theoretical and Applied Genetics, 2012, 125, 133.

Examples of soybeans that have acquired resistance to diseases by a genome-editing technique include a soybean wherein a RXLR effector gene (Avr4/6) is disrupted by using CRISPR-Cas9, thereby shows resistance to soybean Phytophthora root rot caused by Phytophthora sojae (see Mol. Plant. Pathol., 2016, 17, 127.).

Also, there are soybeans that have acquired resistance to soybean diseases other than soybean rust (for example, frogeye leaf spot, target spot, Phytophthora root rot, and sudden death syndrome).

Examples of soybeans in which the product quality is modified by a gene-recombination technique include a soybean “Plenish (trademark)” or “Treus (trademark)” in which a partial gene “gm-fad2-1” of ω-6 desaturase that is a desaturase of fatty acid derived from Glycine max is introduced, thereby the expression of said gene is suppressed, and the oleic acid content is increased; a soybean “Vistive Gold (trademark)” in which a gene that produces a double-stranded RNA of an acyl-acyl carrier protein thioesterase gene “fatb1-A” derived from Glycine max and a gene that produces a double-stranded RNA of a δ-12 desaturase gene “fad2-1A” derived from Glycine max are introduced, thereby the saturated fatty acid content is decreased; a soybean in which a δ-6 desaturase gene “Pj.D6D” derived from Primula juliae and a δ-12 desaturase gene “Nc.Fad3” derived from Neurospora crassa are introduced, thereby a ω3 fatty acid, stearidonic acid is produced; a soybean in which the oil content is modified; a soybean in which the allergen content is decreased (see U.S. Pat. No. 6,864,362); a soybean in which the lysine content is increased (see Bio/Technology, 1995, 13, 577.); a soybean in which the composition of methionine, leucine, isoleucine, and valine is modified; a soybean in which the sulfur amino acid content is increased (see WO 1997/041239 pamphlet); a soybean in which the phenolic compound content is modified (see US Patent Application Publication No. 2008/235829); and a soybean in which the vitamin E content is increased (see WO 2004/058934 pamphlet).

Examples of soybeans in which the product quality is modified by a genome breeding technique include a soybean “Yumeminori” in which the allergen content is decreased.

Examples of soybeans in which a character pertaining to plant growth or yield is modified include a soybean in which a gene “bbx32” encoding a transcription factor regulating the circadian derived from Arabidopsis thaliana is introduced, thereby the plant growth is enhanced, and as a result, a high yield is expected.

Examples of soybeans having other characteristics include a soybean in which the phosphorus uptake is improved; a soybean that has acquired the fertility characters; a soybean that has acquired resistance to drought; a soybean that has acquired resistance to low temperature; a soybean that has acquired resistance to high salinity; a soybean in which the iron chlorosis is improved; and a soybean in which the chloride sensitivity is modified.

The above soybeans also encompass soybeans that have acquired two or more of the above-mentioned resistance to herbicides, resistance to pests, resistance to diseases, resistance to abiotic stresses, characters pertaining to growth or yield, characters pertaining to nutrient uptake, characters pertaining to product quality, fertility characters, and the like. Examples thereof include resistance to glyphosate; resistance to glufosinate;

resistance to frogeye leaf spot, sudden death syndrome, southern stem canker, Phytophthora root rot, southern root-knot nematode, Sclerotinia white mold, brown stem rot, and soybean cyst nematode; improvement of iron chlorosis, and a soybean “Credenz (registered trademark) soybean” in which the chloride sensitivity is modified.

Hereinafter, commercially available or developed soybeans are recited. The following soybeans are represented by [Event Name, Event code, Tread name]. Also, the symbol of “NA” means no information or unavailable information. Many of these soybeans are listed in a registration database (GM APPROVAL DATABASE) in a website (http://www.isaaa.org/) of the INTERNATINAL SERVICE for the ACQUISITION of AGRI-BIOTECH APPLICATIONS (ISAAA).

[260-05 (G94-1, G94-19, G168), DD-026005-3, NA], [A2704-12, ACS-GM005-3, Liberty Link (trademark) soybean], [A2704-21, ACS-GM004-2, Liberty Link (trademark) soybean], [A5547-127, ACS-GM006-4, Liberty Link (trademark) soybean], [A5547-35, ACS-GM008-6, Liberty Link (trademark) soybean], [CV127, BPS-CV127-9, Cultivance], [DAS44406-6, DAS-44406-6, NA], [DAS68416-4, DAS-68416-4, Enlist (trademark) Soybean], [DAS68416-4×MON89788, DAS-68416-4×MON-89788-1, NA], [DAS81419, DAS-81419-2, NA], [DAS81419×DAS44406-6, DAS-81419-2×DAS-44406-6, NA], [DP305423, DP-305423-1, Treus (trademark) or Plenish (trademark)], [DP305423×GTS40-3-2, DP-305423-1×MON-04032-6, NA], [DP356043, DP-356043-5, Optimum GAT (trademark)], [FG72(FG072-2, FG072-3), MST-FG072-3, NA], [FG72×A5547-127, MST-FG072-3×ACS-GM006-4, NA], [GTS40-3-2(40-3-2), MON-04032-6, Roundup Ready (trademark) soybean], [GU262, ACS-GM003-1, Liberty Link (trademark) soybean], [IND-00410-5, IND-00410-5, Verdeca HB4 Soybean], [M0N87701, MON-87701-2, NA], [MON87701×MON89788, MON-87701-2×MON-89788-1, Intacta (trademark) Roundup Ready (trademark) 2 Pro], [M0N87705, MON-87705-6, Vistive Gold (trademark)], [MON87705×MON87708, MON-87705-6×MON-87708-9, NA], [MON87705×MON87708×MON89788, MON-87705-6×MON-87708-9×MON-89788-1, NA], [MON87705×MON89788, MON-87705-6×MON-89788-1, NA], [M0N87708, MON-87708-9, Genuity (registered trademark) Roundup Ready (trademark) 2 Xtend (trademark)], [MON87708×MON89788, MON-87708-9×MON-89788-1, Roundup Ready 2 Xtend (registered trademark)], [M0N87712, MON-87712-4, NA], [M0N87751, MON-87751-7, NA], [MON87751×MON87701×MON87708×MON89788, MON-87751-7×MON-87701-2×MON87708×MON89788, NA], [M0N87769, M0N87769-7, NA], [, MON87769×MON89788, MON-87769-7×MON-89788-1, NA], [M0N89788, MON-89788-1, Genuity (registered trademark) Roundup Ready 2 Yield (trademark)], [SYHT0H2, SYN-000H2-5, Herbicide-tolerant Soybean line], [W62, ACS-GM002-9, Liberty Link (trademark) soybean], [W98, ACS-GM001-8, Liberty Link (trademark) soybean], [OT96-15, OT96-15, NA], [NA, NA, STS (registered trademark) soybean], [NA, NA, Credenz (registered trademark) soybean], [NA, NA, Enlist E3 (trademark)], [NA, NA, Enlist (trademark) Roundup Ready 2 Yield (registered trademark)], [NA, NA, Fukuminori], [NA, NA, Yumeminori], [DP305423×M0V87708, DP-305423-1×MON-87708-9, NA], [DP305423×M0V87708×M0N89788, DP-305423-1×MON-87708-9×MON-89788-1, NA], [DP305423×M0N89788, DP-305423-1×MON-89788-1, NA]

Applying the Present compound, the Compound of the present invention, or the Composition A to a soybean achieves effects for promoting the plant growth such as the increase in the rate of seedling establishment, increase in the number of healthy leaves, increase in the height of the plant, increase in the weight of the plant, increase in the leaf area, increase in the number or weight of seeds, increase in the number of occasion of flower setting or fruit setting, and promotion in the growth of a root. Also, applying the Present compound, the Compound of the present invention, or the Composition A to a soybean achieves the improvement in resistance to abiotic stresses such as temperature stresses (for example, high temperature stress and low temperature stress), water stresses (for example, drought stress and excess water stress), or salt stresses.

### EXAMPLES

Hereinafter, the present invention is illustrated more in detail by Preparation Examples, Reference Preparation Examples, Formulation Examples, and Test Examples, but the present invention is not limited to these Examples only.

In the present description, Et represents an ethyl group, Pr represents a propyl group, i-Pr represents an isopropyl group, c-Pr represents a cyclopropyl group, Bu represents a butyl group, and Ph represents a phenyl group.

First, Preparation Examples of the Present compound are shown below.

### Reference Preparation Example 1

To a mixture of 6-bromoindazole (5.91 g), sodium hydride (60%, oily) (1.44 g), and DMF (40 mL) was added 2-bromoacetic acid (5.51 g) under ice-cooling, and the resulting mixture was stirred at 60° C. for 4 hours. To the resulting mixture was added water, and the resulting mixture was extracted with ethyl acetate. The resulting organic layer was sequentially washed with water and saturated brine, dried over anhydrous sodium sulfate, and concentrated under reduced pressure to give the Intermediate compound 1 represented by the following formula (6.01 g).

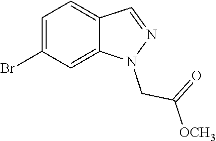 [SMILES: COC(=O)Cn1ncc2ccc(Br)cc21]

Intermediate compound 1: ¹H-NMR (CDCl₃) δ: 8.02 (1H, d), 7.61 (1H, dd), 7.53 (1H, br s), 7.29 (1H, dd), 5.13 (2H, s), 3.77 (3H, s).

### Preparation Example 1

To a mixture of sodium hydride (60%, oily) (0.89 g), methyl formate (3.35 g), and DMF (30 mL) was added the Intermediate compound 1 (5.00 g) under ice-cooling, and the resulting mixture was stirred at room temperature for 4 hours. To the resulting mixture was added water, and the resulting mixture was extracted with ethyl acetate. The resulting organic layer was sequentially washed with water and saturated brine, dried over anhydrous sodium sulfate, and concentrated under reduced pressure. To the resulting crude product were added potassium carbonate (3.08 g), DMF (30 mL), and dimethyl sulfate (2.81 g), and the resulting mixture was stirred at room temperature for 4 hours. To the resulting mixture was added water, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography (hexane:ethyl acetate=1:1) to give the Present compound 1 represented by the following formula (2.10 g).

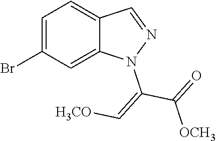 [SMILES: CO/C=C(/C(=O)OC)n1ncc2ccc(Br)cc21]

Present compound 1: ¹H-NMR (CDCl₃) δ: 8.14 (1H, s), 7.82 (1H, s), 7.61 (1H, d), 7.38 (1H, d), 7.29 (1H, dd), 3.93 (3H, s), 3.74 (3H, s).

### Preparation Example 2

A mixture of the Present compound 1 (0.31 g), cyclopropylacetylene (0.66 g), bis(acetonitrile)dichloropalladium(II) (0.01 g), XPhos (0.05 g), cesium carbonate (0.85 g), and acetonitrile (5 mL) was stirred at 80° C. for 6 hours. To the resulting mixture was added water, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography (hexane:ethyl acetate=1:1) to give the Present compound 2 represented by the following formula (0.15 g).

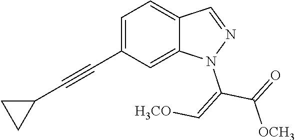 [SMILES: CO/C=C(/C(=O)OC)n1ncc2ccc(C#CC3CC3)cc21]

Present compound 2: ¹H-NMR (CDCl₃) δ: 8.12 (1H, d), 7.81 (1H, s), 7.63 (1H, dd), 7.24 (1H, d), 7.17 (1H, dd), 3.90 (3H, s), 3.71 (3H, s), 1.50-1.43 (1H, m), 0.91-0.81 (4H, m).

### Preparation Example 3

A mixture of the Present compound 1 (0.93 g), phenylboronic acid (0.55 g), {1,1′-bis(diphenylphosphino)ferrocene}dichloropalladium(II) (0.22 g), tripotassium phosphate (1.91 g), DME (10 mL), and water (0.1 mL) was stirred under reflux for 6 hours. To the resulting mixture was added water, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography (hexane:ethyl acetate=1:1) to give the Present compound 3 represented by the following formula (0.44 g).

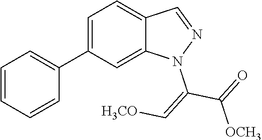 [SMILES: CO/C=C(/C(=O)OC)n1ncc2ccc(-c3ccccc3)cc21]

Present compound 3: ¹H-NMR (CDCl₃) δ: 8.19 (1H, s), 7.85 (1H, s), 7.80 (1H, d), 7.64 (2H, d), 7.49-7.42 (3H, m), 7.40-7.34 (2H, m), 3.91 (3H, s), 3.74 (3H, s).

### Preparation Example 4

To a mixture of the Present compound 12 (0.15 g) and DMF (2 mL) was added N-chlorosuccinimide (0.08 g), and the resulting mixture was stirred at 60° C. for 2 hours. To the resulting mixture was added a 1 M aqueous solution of sodium thiosulfate, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography (ethyl acetate:hexane=1:2) to give the Present compound 13 represented by the following formula (0.13 g) and the Present compound 14 represented by the following formula (0.03 g).

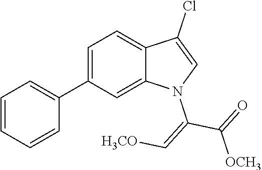 [SMILES: CO/C=C(/C(=O)OC)n1cc(Cl)c2ccc(-c3ccccc3)cc21]

Present compound 13: ¹H-NMR (CDCl₃) δ: 7.77 (1H, s), 7.68 (1H, d), 7.61 (2H, dd), 7.46-7.42 (3H, m), 7.34 (1H, d), 7.23 (1H, d), 7.03 (1H, s), 3.91 (3H, s), 3.75 (3H, s).

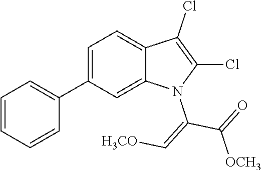 [SMILES: CO/C=C(/C(=O)OC)n1c(Cl)c(Cl)c2ccc(-c3ccccc3)cc21]

Present compound 14: ¹H-NMR (CDCl₃) δ: 7.90 (1H, s), 7.64-7.58 (3H, m), 7.48-7.41 (3H, m), 7.34 (1H, d), 7.21 (1H, d), 3.92 (3H, s), 3.73 (3H, s).

### Preparation Example 5-1

A mixture of the Present compound 10 (4.56 g) described in the Preparation Example 7, palladium(II) acetate (0.34 g), triethylsilane (3.56 g), 1,4-bis(diphenylphosphino)butane (0.98 g), sodium carbonate (2.39 g), N-formylsaccharin (4.88 g), and DMF (40 mL) was stirred at 80° C. for 6 hours. To the resulting mixture was added water, and the resulting mixture was extracted with ethyl acetate. The resulting organic layer was washed with saturated brine, dried over anhydrous sodium sulfate, and concentrated under reduced pressure. The resulting residue was subjected to silica gel column chromatography to give the Intermediate compound 2 represented by the following formula (1.35 g).

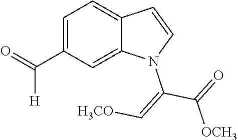 [SMILES: CO/C=C(/C(=O)OC)n1ccc2ccc(C=O)cc21]

Intermediate compound 2: ¹H-NMR (CDCl₃) δ: 7.74 (1H, s), 7.63 (1H, dq), 7.19 (1H, td), 7.14-7.10 (1H, m), 7.08 (1H, dd), 7.03 (1H, d), 6.62 (1H, dd), 3.88 (3H, s), 3.73 (3H, s).

### Preparation Example 5-2

A mixture of the Intermediate compound 2 (1.25 g), hydroxylamine hydrochloride (0.36 g), pyridine (0.82 mL), and methanol (15 mL) was stirred at room temperature for 5 hours. To the resulting mixture was added water, and the resulting mixture was extracted with ethyl acetate. The resulting organic layer was washed with saturated brine, dried over anhydrous sodium sulfate, and concentrated under reduced pressure. The resulting residue was subjected to silica gel column chromatography to give the Intermediate compound 3 represented by the following formula (1.30 g).

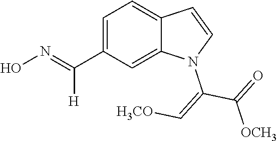 [SMILES: CO/C=C(/C(=O)OC)n1ccc2ccc(/C=N/O)cc21]

Intermediate compound 3: ¹H-NMR (CDCl₃) δ: 8.22 (1H, s), 7.76 (1H, s), 7.62 (1H, d), 7.38 (1H, dd), 7.29 (1H, d), 7.17 (1H, s), 7.09 (1H, d), 6.62 (1H, d), 3.89 (3H, s), 3.74 (3H, s).

### Preparation Example 5-3

A mixture of the Intermediate compound 3 (0.12 g), methyl iodide (0.18 g), cesium carbonate (0.21 g), and N-methylpyrrolidone (3 mL) was stirred at room temperature for 5 hours. To the resulting mixture was added water, and the resulting mixture was extracted with MTBE. The resulting organic layer was washed with saturated brine, dried over anhydrous sodium sulfate, and concentrated under reduced pressure. The resulting residue was subjected to silica gel column chromatography (hexane:ethyl acetate=2:1) to give the Present compound 36 represented by the following formula (0.10 g).

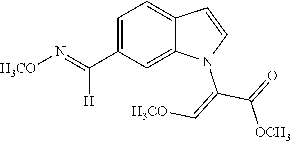 [SMILES: CO/C=C(/C(=O)OC)n1ccc2ccc(/C=N/OC)cc21]

Present compound 36: ¹H-NMR (CDCl₃) δ: 8.15 (1H, s), 7.76 (1H, s), 7.60 (1H, d), 7.39 (1H, dd), 7.27 (1H, d), 7.07 (1H, d), 6.61 (1H, dd), 3.97 (3H, s), 3.89 (3H, s), 3.73 (3H, s).

### Preparation Example 6-1

A mixture of the Present compound 10 (3.10 g), bis(triphenylphosphine)palladium(II) dichloride (0.35 g), tributyl(1-ethoxyvinyl)tin (4.1 mL), and DME (20 mL) was stirred under reflux for 2 days. To the resulting mixture was added 1N hydrochloric acid (8 mL), and the resulting mixture was stirred at room temperature for 1 hour. The resulting mixture was concentrated under reduced pressure, and extracted with ethyl acetate. The resulting organic layer was concentrated under reduced pressure. The resulting residue was subjected to silica gel column chromatography (ethyl acetate:hexane=1:2) to give the Intermediate compound 4 represented by the following formula (0.76 g).

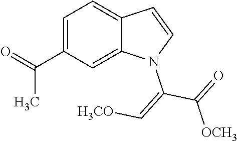 [SMILES: CO/C=C(/C(=O)OC)n1ccc2ccc(C(C)=O)cc21]

Intermediate compound 4: ¹H-NMR (CDCl₃) δ: 7.80-7.77 (2H, m), 7.75 (1H, d), 7.66 (1H, d), 7.20 (1H, d), 6.66 (1H, d), 3.90 (3H, s), 3.75 (3H, s), 2.65 (3H, s).

### Preparation Example 6-2

The Intermediate compound 5 represented by the following formula was prepared according to the Preparation Example 5-2 by using the Intermediate compound 4 instead of the Intermediate compound 2.

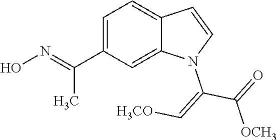 [SMILES: CO/C=C(/C(=O)OC)n1ccc2ccc(/C(C)=N/O)cc21]

Intermediate compound 5: ¹H-NMR (CDCl₃) δ: 7.76 (1H, s), 7.61 (1H, d), 7.43 (1H, dd), 7.35 (1H, s), 7.09 (1H, br s), 7.07 (1H, d), 6.61 (1H, dd), 3.89 (3H, s), 3.73 (3H, s), 2.33 (3H, s).

### Preparation Example 6-3

The Present compound 42 represented by the following formula was prepared according to the Preparation Example 5-3 by using the Intermediate compound 5 instead of the Intermediate compound 3.

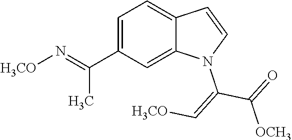 [SMILES: CO/C=C(/C(=O)OC)n1ccc2ccc(/C(C)=N/OC)cc21]

Present compound 42: ¹H-NMR (CDCl₃) δ: 7.76 (1H, d), 7.59 (1H, d), 7.46 (1H, dd), 7.32 (1H, d), 7.06 (1H, d), 6.60 (1H, d), 4.00 (3H, s), 3.89 (3H, s), 3.73 (3H, s), 2.27 (3H, s).

### Preparation Example 7

The compounds prepared according to the methods described in the Production methods, Preparation Examples, or the like, and physical properties thereof are shown below.

A compound represented by formula (I):

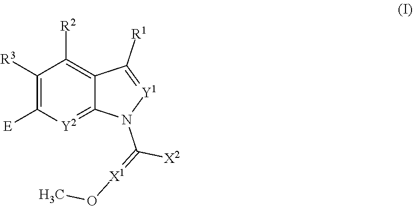

wherein the combination of E, X¹, X², Y¹, R¹, R², R³, and Y² represents any one combination indicated in Table T1 to Table T6.

The term of “Comp” described in Table T1 to Table T6 means the Present compound number.

E represents any one of substituent numbers 1 to 216 described in Table V1 to Table V7. The term of “sub number” described in Table V1 to Table V7 means the substituent number.

For example, the compound wherein Comp (Present compound number) is 4 described in Table T1, namely the Present compound 4 means a compound wherein E represents the group of sub number (substituent number) 13 described in Table V1, X¹ represents CH, X² represents C(O)OCH₃, Y¹ and R¹ are combined with the carbon atom to which they are attached to form a benzene ring, R² represents a hydrogen atom, R³ represents a hydrogen atom, and Y² represents CH. Specifically, the Present compound 4 is a compound of the following structure.

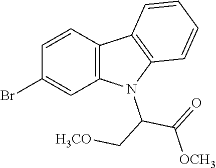 [SMILES: COCC(C(=O)OC)n1c2ccccc2c2ccc(Br)cc21]

TABLE V1

| sub | |
|---|---|
| number | E |
| 1 | 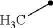 |
| 2 | 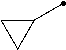 |
| 3 | 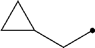 |
| 4 | 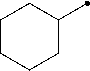 |
| 5 | 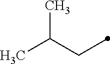 |
| 6 | 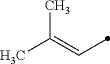 |
| 7 | 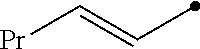 |
| 8 | 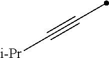 |
| 9 | 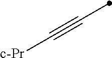 |
| 10 | 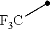 |
| 11 | 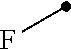 |
| 12 | 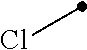 |
| 13 | 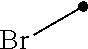 |
| 14 | 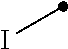 |
| 15 | 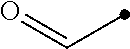 |
| 16 | 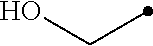 |
| 17 | 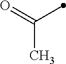 |
| 18 | 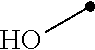 |
| 19 | 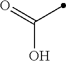 |
| 20 | 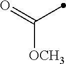 |
| 21 | 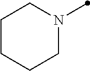 |
| 22 | 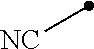 |
| 23 | 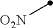 |
| 24 | 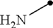 |
| 25 | 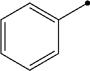 |
| 26 | 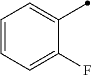 |
| 27 | 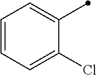 |
| 28 | 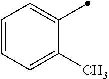 |
| 29 | 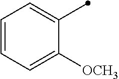 |
| 30 | 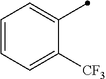 |

TABLE V2

| sub | |
|---|---|
| number | E |
| 32 | 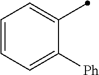 |
| 32 | 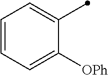 |
| 33 | 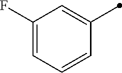 |
| 34 | 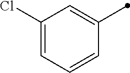 |
| 35 | 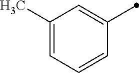 |
| 36 | 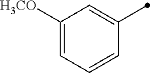 |
| 37 | 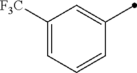 |
| 38 | 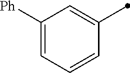 |
| 39 | 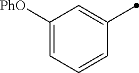 |
| 40 | 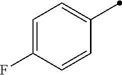 |
| 41 | 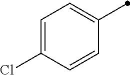 |
| 42 | 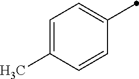 |
| 43 | 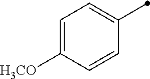 |
| 44 | 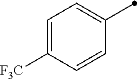 |
| 45 | 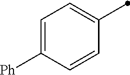 |
| 46 | 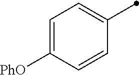 |
| 47 | 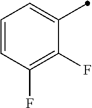 |
| 48 | 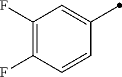 |
| 49 | 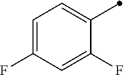 |
| 50 | 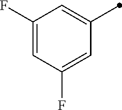 |
| 51 | 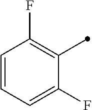 |
| 52 | 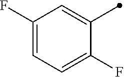 |
| 53 | 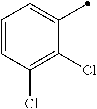 |
| 54 | 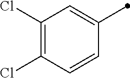 |
| 55 | 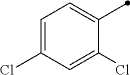 |
| 56 | 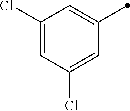 |
| 57 | 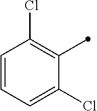 |
| 58 | 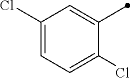 |
| 59 | 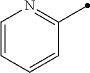 |
| 60 | 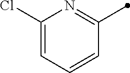 |

TABLE V3

| sub | |
|---|---|
| number | E |
| 61 | 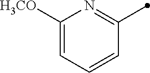 |
| 62 | 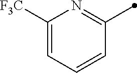 |
| 63 | 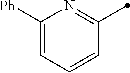 |
| 64 | 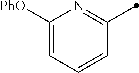 |
| 65 | 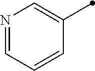 |
| 66 | 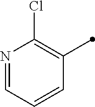 |
| 67 | 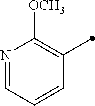 |
| 68 | 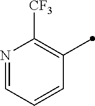 |
| 69 | 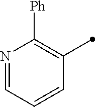 |
| 70 | 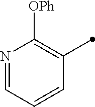 |
| 71 | 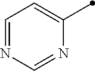 |
| 72 | 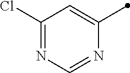 |
| 73 | 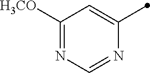 |
| 74 | 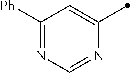 |
| 75 | 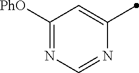 |
| 76 | 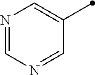 |
| 77 | 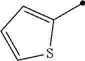 |
| 78 | 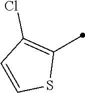 |
| 79 | 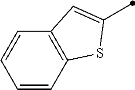 |
| 80 | 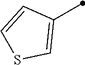 |
| 81 | 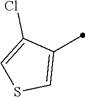 |
| 82 | 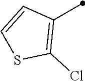 |
| 83 | 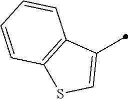 |
| 84 | 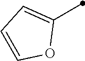 |
| 85 | 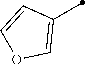 |
| 86 | 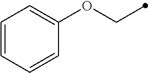 |
| 87 | 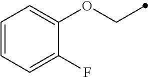 |
| 88 | 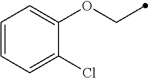 |
| 89 | 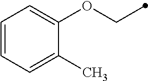 |
| 90 | 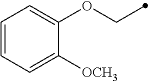 |

TABLE V4

| sub | |
|---|---|
| number | E |
| 91 | 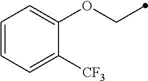 |
| 92 | 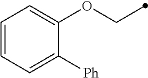 |
| 93 | 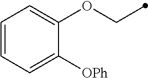 |
| 94 | 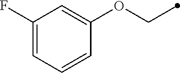 |
| 95 | 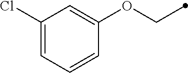 |
| 96 | 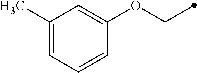 |
| 97 | 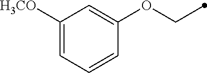 |
| 98 | 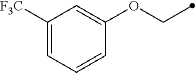 |
| 99 | 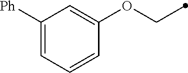 |
| 100 | 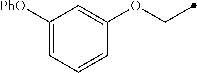 |
| 101 | 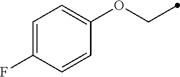 |
| 102 | 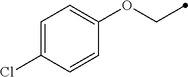 |
| 103 | 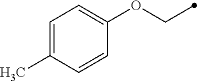 |
| 104 | 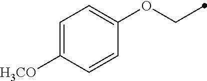 |
| 105 | 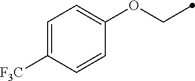 |
| 106 | 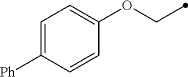 |
| 107 | 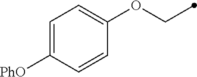 |
| 108 | 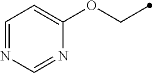 |
| 109 | 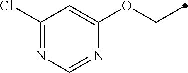 |
| 110 | 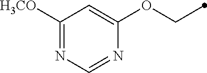 |
| 111 | 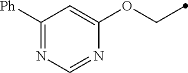 |
| 112 | 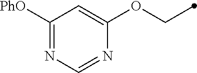 |
| 113 | 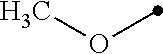 |
| 114 | 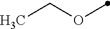 |
| 115 | 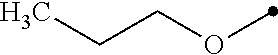 |
| 116 | 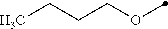 |
| 117 | 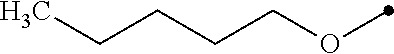 |
| 118 | 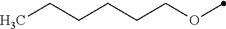 |
| 119 | 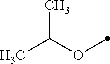 |
| 120 | 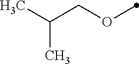 |

TABLE V5

| sub | |
|---|---|
| number | E |
| 121 | 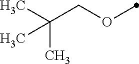 |
| 122 | 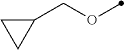 |
| 123 | 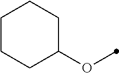 |
| 124 | 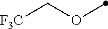 |
| 125 | 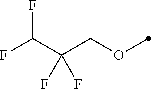 |
| 126 | 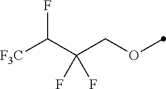 |
| 127 | 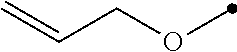 |
| 128 | 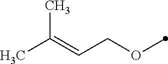 |
| 129 | 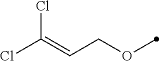 |
| 130 | 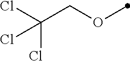 |
| 131 | 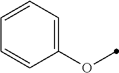 |
| 132 | 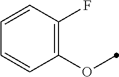 |
| 133 | 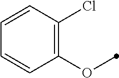 |
| 134 | 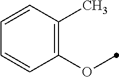 |
| 135 | 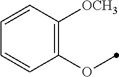 |
| 136 | 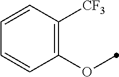 |
| 137 | 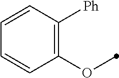 |
| 138 | 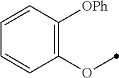 |
| 139 | 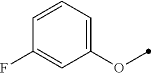 |
| 140 | 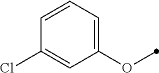 |
| 141 | 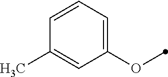 |
| 142 | 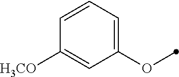 |
| 143 | 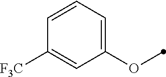 |
| 144 | 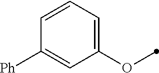 |
| 145 | 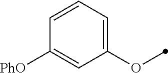 |
| 146 | 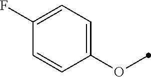 |
| 147 | 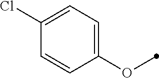 |
| 148 | 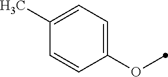 |
| 149 | 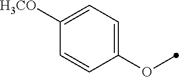 |
| 150 | 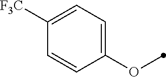 |

TABLE V6

| sub | |
|---|---|
| number | E |
| 151 | 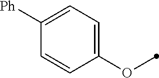 |
| 152 | 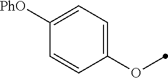 |
| 153 | 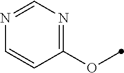 |
| 154 | 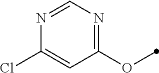 |
| 155 | 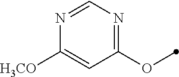 |
| 156 | 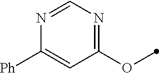 |
| 157 | 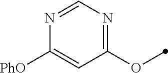 |
| 158 | 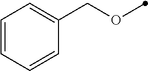 |
| 159 | 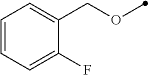 |
| 160 | 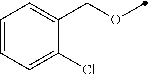 |
| 161 | 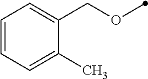 |
| 162 | 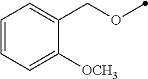 |
| 163 | 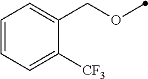 |
| 164 | 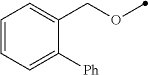 |
| 165 | 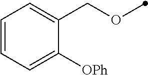 |
| 166 | 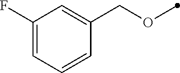 |
| 167 | 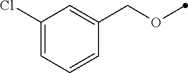 |
| 168 | 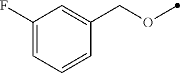 |
| 169 | 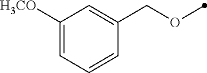 |
| 170 | 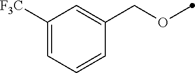 |
| 171 | 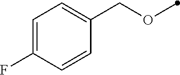 |
| 172 | 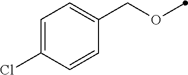 |
| 173 | 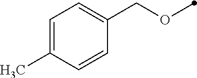 |
| 174 | 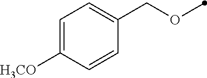 |
| 175 | 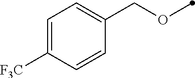 |
| 176 | 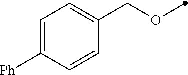 |
| 177 | 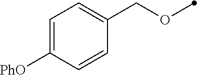 |
| 178 | 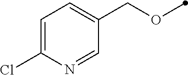 |
| 179 | 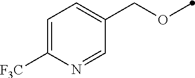 |
| 180 | 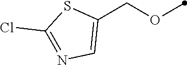 |

TABLE V7

| sub | |
|---|---|
| number | E |
| 181 | 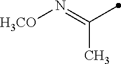 |
| 182 | 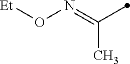 |
| 183 | 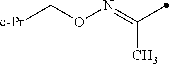 |
| 184 | 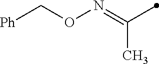 |
| 185 | 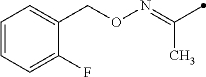 |
| 186 | 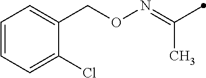 |
| 187 | 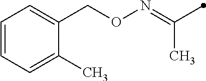 |
| 188 | 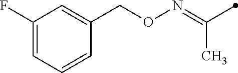 |
| 189 | 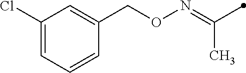 |
| 190 | 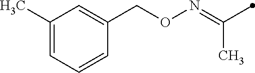 |
| 191 | 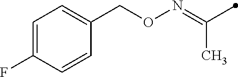 |
| 192 | 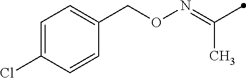 |
| 193 | 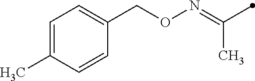 |
| 194 | 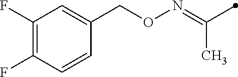 |
| 195 | 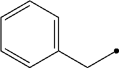 |
| 196 | 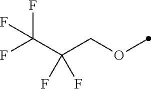 |
| 197 | 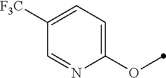 |
| 198 | 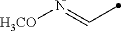 |
| 199 | 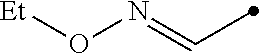 |
| 200 | 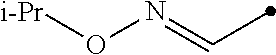 |
| 201 | 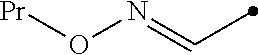 |
| 202 | 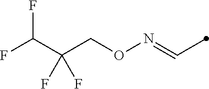 |
| 203 | 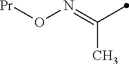 |
| 204 | 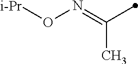 |
| 205 | 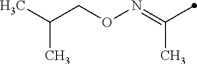 |
| 206 | 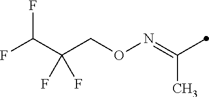 |
| 207 | 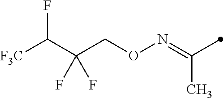 |
| 208 | 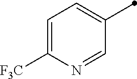 |
| 209 | 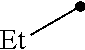 |
| 210 | 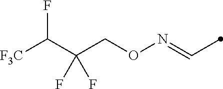 |
| 211 | 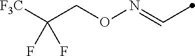 |
| 212 | 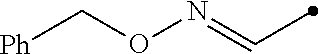 |
| 213 | 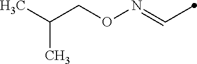 |
| 214 | 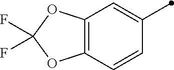 |
| 215 | 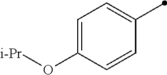 |
| 216 | 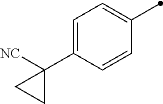 |
| 217 | 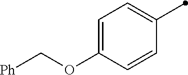 |
| 218 | 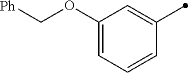 |
| 219 | 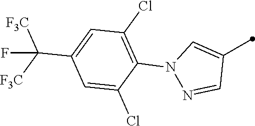 |
| 220 | 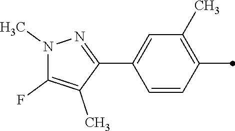 |
| 221 | 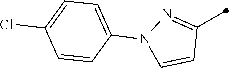 |

TABLE T1

| Comp | E | X¹ | X² | Y¹ | R¹ | R² | R³ | Y² |
|---|---|---|---|---|---|---|---|---|
| 1 | 13 | CH | C (O) OCH₃ | N | H | H | H | CH |
| 2 | 9 | CH | C (O) OCH₃ | N | H | H | H | CH |
| 3 | 25 | CH | C (O) OCH₃ | N | H | H | H | CH |
| 4 | 13 | CH | C (O) OCH₃ | C—CH═CH—CH═CH | | H | H | CH |
| 5 | 9 | CH | C (O) OCH₃ | C—CH═CH—CH═CH | | H | H | CH |
| 6 | 25 | CH | C (O) OCH₃ | C—CH═CH—CH═CH | | H | H | CH |
| 7 | 13 | CH | C (O) OCH₃ | CH | H | H | F | CH |
| 8 | 9 | CH | C (O) OCH₃ | CH | H | H | F | CH |
| 9 | 25 | CH | C (O) OCH₃ | CH | H | H | F | CH |
| 10 | 13 | CH | C (O) OCH₃ | CH | H | H | H | CH |
| 11 | 9 | CH | C (O) OCH₃ | CH | H | H | H | CH |
| 12 | 25 | CH | C (O) OCH₃ | CH | H | H | H | CH |
| 13 | 25 | CH | C (O) OCH₃ | CH | Cl | H | H | CH |
| 14 | 25 | CH | C (O) OCH₃ | C (Cl) | Cl | H | H | CH |

TABLE T2

| Comp | E | X¹ | X² | Y¹ | R¹ | R² | R³ | Y² |
|---|---|---|---|---|---|---|---|---|
| 15 | 29 | CH | C (O) OCH₃ | CH | H | H | H | CH |
| 16 | 42 | CH | C (O) OCH₃ | CH | H | H | H | CH |
| 17 | 36 | CH | C (O) OCH₃ | CH | H | H | H | CH |
| 18 | 26 | CH | C (O) OCH₃ | CH | H | H | H | CH |
| 19 | 35 | CH | C (O) OCH₃ | CH | H | H | H | CH |
| 20 | 33 | CH | C (O) OCH₃ | CH | H | H | H | CH |
| 21 | 43 | CH | C (O) OCH₃ | CH | H | H | H | CH |
| 22 | 40 | CH | C (O) OCH₃ | CH | H | H | H | CH |
| 23 | 28 | CH | C (O) OCH₃ | CH | H | H | H | CH |
| 24 | 79 | CH | C (O) OCH₃ | CH | H | H | H | CH |
| 25 | 80 | CH | C (O) OCH₃ | CH | H | H | H | CH |
| 26 | 77 | CH | C (O) OCH₃ | CH | H | H | H | CH |
| 27 | 2 | CH | C (O) OCH₃ | CH | H | H | H | CH |
| 28 | 196 | CH | C (O) OCH₃ | CH | H | H | H | CH |

TABLE T3

| Comp | E | X¹ | X² | Y¹ | R¹ | R² | R³ | Y² |
|---|---|---|---|---|---|---|---|---|
| 29 | 114 | CH | C (O) OCH₃ | CH | H | H | H | CH |
| 30 | 83 | CH | C (O) OCH₃ | CH | H | H | H | CH |
| 31 | 13 | CH | C (O) OCH₃ | CH | H | H | H | N |
| 32 | 18 | CH | C (O) OCH₃ | CH | H | H | H | CH |
| 33 | 131 | CH | C (O) OCH₃ | CH | H | H | H | CH |
| 34 | 9 | CH | C (O) OCH₃ | CH | H | H | H | N |
| 35 | 197 | CH | C (O) OCH₃ | CH | H | H | H | CH |
| 36 | 198 | CH | C (O) OCH₃ | CH | H | H | H | CH |
| 37 | 199 | CH | C (O) OCH₃ | CH | H | H | H | CH |
| 38 | 200 | CH | C (O) OCH₃ | CH | H | H | H | CH |
| 39 | 201 | CH | C (O) OCH₃ | CH | H | H | H | CH |
| 40 | 202 | CH | C (O) OCH₃ | CH | H | H | H | CH |
| 41 | 13 | CH | C (O) OCH₃ | N | Cl | H | H | CH |
| 42 | 181 | CH | C (O) OCH₃ | CH | H | H | H | CH |

TABLE T4

| Comp | E | X¹ | X² | Y¹ | R¹ | R² | R³ | Y² |
|---|---|---|---|---|---|---|---|---|
| 43 | 182 | CH | C (O) OCH₃ | CH | H | H | H | CH |
| 44 | 203 | CH | C (O) OCH₃ | CH | H | H | H | CH |
| 45 | 204 | CH | C (O) OCH₃ | CH | H | H | H | CH |
| 46 | 205 | CH | C (O) OCH₃ | CH | H | H | H | CH |
| 47 | 184 | CH | C (O) OCH₃ | CH | H | H | H | CH |
| 48 | 206 | CH | C (O) OCH₃ | CH | H | H | H | CH |
| 49 | 207 | CH | C (O) OCH₃ | CH | H | H | H | CH |
| 50 | 208 | CH | C (O) NHCH₃ | C (CH₃) | H | H | H | CH |
| 51 | 1 | CH | C (O) OCH₃ | CH | H | H | H | CH |
| 52 | 209 | CH | C (O) OCH₃ | CH | H | H | H | CH |
| 53 | 210 | CH | C (O) OCH₃ | CH | H | H | H | CH |
| 54 | 211 | CH | C (O) OCH₃ | CH | H | H | H | CH |
| 55 | 212 | CH | C (O) OCH₃ | CH | H | H | H | CH |
| 56 | 213 | CH | C (O) OCH₃ | CH | H | H | H | CH |

TABLE T5

| Comp | E | X¹ | X² | Y¹ | R¹ | R² | R³ | Y² |
|---|---|---|---|---|---|---|---|---|
| 57 | 13 | CH | C (O) OCH₃ | CH | H | H | CH₃ | CH |
| 58 | 181 | CH | C (O) OCH₃ | CH | H | H | CH₃ | CH |
| 59 | 182 | CH | C (O) OCH₃ | CH | H | H | CH₃ | CH |
| 60 | 203 | CH | C (O) OCH₃ | CH | H | H | CH₃ | CH |
| 61 | 13 | N | C (O) NHCH₃ | CH | H | H | H | CH |
| 62 | 44 | N | C (O) NHCH₃ | CH | H | H | H | CH |
| 63 | 13 | CH | C (O) OCH₃ | CH | CN | H | H | CH |
| 64 | 13 | CH | C (O) OCH₃ | C (CN) | H | H | H | CH |
| 65 | 13 | CH | C (O) OCH₃ | C (CH₃) | H | H | H | CH |
| 66 | 37 | CH | C (O) OCH₃ | CH | CN | H | H | CH |
| 67 | 44 | CH | C (O) OCH₃ | CH | CN | H | H | CH |
| 68 | 214 | CH | C (O) OCH₃ | C (CN) | H | H | H | CH |
| 69 | 215 | CH | C (O) OCH₃ | C (CN) | H | H | H | CH |
| 70 | 208 | CH | C (O) OCH₃ | C (CH₃) | H | H | H | CH |

TABLE T6

| Comp | E | X¹ | X² | Y¹ | R¹ | R² | R³ | Y² |
|---|---|---|---|---|---|---|---|---|
| 71 | 216 | CH | C (O) OCH₃ | C (CH₃) | H | H | H | CH |
| 72 | 13 | N | C (O) OCH₃ | CH | H | H | H | CH |
| 73 | 208 | CH | C (O) NHCH₃ | CH | H | H | H | CH |
| 74 | 217 | CH | C (O) OCH₃ | CH | H | H | H | CH |
| 75 | 218 | CH | C (O) OCH₃ | CH | H | H | H | CH |
| 76 | 219 | CH | C (O) OCH₃ | CH | H | H | H | CH |
| 77 | 220 | CH | C (O) OCH₃ | CH | H | H | H | CH |
| 78 | 221 | CH | C (O) OCH₃ | CH | H | H | H | CH |
| 79 | 13 | N | 5,6-dihydro- | CH | H | H | H | CH |
| | | | 1,4,2-dioxazin- | | | | | |
| | | | 3-yl | | | | | |

Present compound 4: ¹H-NMR (CDCl₃) δ: 8.04 (1H, d), 7.94 (1H, s), 7.92 (1H, d), 7.43 (1H, ddd), 7.35 (1H, dd), 7.30 (1H, d), 7.28-7.24 (1H, m), 7.15 (1H, d), 3.88 (3H, s), 3.69 (3H, s).

Present compound 5: ¹H-NMR (CDCl₃) δ: 8.03 (1H, dq), 7.96 (1H, dd), 7.93 (1H, s), 7.40 (1H, ddd), 7.29-7.19 (3H, m), 7.14 (1H, dt), 3.85 (3H, s), 3.66 (3H, s), 1.53-1.45 (1H, m), 0.91-0.81 (4H, m).

Present compound 6: ¹H-NMR (CDCl₃) δ: 8.13 (1H, dd), 8.10 (1H, dt), 7.97 (1H, s), 7.69 (2H, dt), 7.51-7.24 (7H, m), 7.17 (1H, d), 3.87 (3H, s), 3.68 (3H, s).

Present compound 7: ¹H-NMR (CDCl₃) δ: 7.74 (1H, s), 7.34 (1H, d), 7.23 (1H, d), 7.05 (1H, d), 6.55 (1H, d), 3.91 (3H, s), 3.75 (3H, s).

Present compound 8: ¹H-NMR (CDCl₃) δ: 7.72 (1H, s), 7.23 (1H, d), 7.07 (1H, d), 7.05 (1H, d), 6.52 (1H, dd), 3.88 (3H, s), 3.72 (3H, s), 1.53-1.47 (1H, m), 0.89-0.83 (4H, m).

Present compound 9: ¹H-NMR (CDCl₃) δ: 7.74 (1H, s), 7.59-7.55 (2H, m), 7.45-7.41 (2H, m), 7.38-7.32 (2H, m), 7.08 (1H, d), 7.06 (1H, d), 6.59 (1H, dd), 3.89 (3H, s), 3.74 (3H, s).

Present compound 10: ¹H-NMR (CDCl₃) δ: 7.74 (1H, s), 7.48 (1H, d), 7.23-7.20 (2H, m), 6.99 (1H, d), 6.58 (1H, d), 3.90 (3H, s), 3.74 (3H, s).

Present compound 11: ¹H-NMR (CDCl₃) δ: 7.73 (1H, s), 7.50 (1H, d), 7.15-7.13 (2H, m), 7.02 (1H, d), 6.57 (1H, dd), 3.87 (3H, s), 3.71 (3H, s), 1.49-1.43 (1H, m), 0.89-0.77 (4H, m).

Present compound 12: ¹H-NMR (CDCl₃) δ: 7.77 (1H, s), 7.68 (1H, d), 7.63 (2H, d), 7.46-7.36 (3H, m), 7.34-7.26 (2H, m), 7.06 (1H, d), 6.63 (1H, d), 3.89 (3H, s), 3.74 (3H, s).

Present compound 15: ¹H-NMR (CDCl₃) δ: 7.73 (1H, s), 7.63 (1H, dd), 7.37-7.28 (3H, m), 7.21 (1H, m), 7.05-6.98 (3H, m), 6.62 (1H, dd), 3.87 (3H, s), 3.79 (3H, s), 3.72 (3H, s).

Present compound 16: ¹H-NMR (CDCl₃) δ: 7.77 (1H, s), 7.66 (1H, d), 7.53 (2H, d), 7.37 (1H, dd), 7.26-7.22 (3H, m), 7.05 (1H, dd), 6.62 (1H, d), 3.88 (3H, s), 3.73 (3H, s), 2.40 (3H, s).

Present compound 17: ¹H-NMR (CDCl₃) δ: 7.77 (1H, s), 7.67 (1H, d), 7.40-7.32 (2H, m), 7.27 (1H, m), 7.22 (1H, dt), 7.17 (1H, t), 7.06 (1H, d), 6.87 (1H, ddd), 6.63 (1H, dd), 3.89 (3H, s), 3.87 (3H, s), 3.74 (3H, s).

Present compound 18: ¹H-NMR (CDCl₃) δ: 7.75 (1H, s), 7.68 (1H, dd), 7.49 (1H, td), 7.31 (1H, dt), 7.28-7.26 (2H, m), 7.20 (1H, td), 7.14 (1H, ddd), 7.09 (1H, d), 6.64 (1H, dd), 3.90 (3H, s), 3.74 (3H, s).

Present compound 19: ¹H-NMR (CDCl₃) δ: 7.77 (1H, s), 7.67 (1H, dd), 7.46-7.41 (2H, m), 7.37 (1H, dd), 7.32 (1H, t), 7.26 (1H, m), 7.13 (1H, d), 7.05 (1H, d), 6.63 (1H, dd), 3.88 (3H, s), 3.73 (3H, s), 2.43 (3H, s).

Present compound 20: ¹H-NMR (CDCl₃) δ: 7.78 (1H, s), 7.68 (1H, dd), 7.43-7.34 (3H, m), 7.32 (1H, ddd), 7.25-7.24 (1H, m), 7.07 (1H, d), 7.03-6.96 (1H, m), 6.64 (1H, dd), 3.90 (3H, s), 3.75 (3H, s).

Present compound 21: ¹H-NMR (CDCl₃) δ: 7.77 (1H, s), 7.65 (1H, d), 7.56 (2H, d), 7.34 (1H, d), 7.21 (1H, s), 7.04 (1H, d), 6.97 (2H, d), 6.62 (1H, d), 3.89 (3H, s), 3.85 (3H, s), 3.73 (3H, s).

Present compound 22: ¹H-NMR (CDCl₃) δ: 7.77 (1H, s), 7.67 (1H, dd), 7.57 (2H, dd), 7.32 (1H, dd), 7.20 (1H, t), 7.11 (2H, t), 7.06 (1H, d), 6.63 (1H, dd), 3.89 (3H, s), 3.74 (3H, s).

Present compound 23: ¹H-NMR (CDCl₃) δ: 7.71 (1H, s), 7.64 (1H, dd), 7.30-7.22 (4H, m), 7.10 (1H, dd), 7.07 (1H, d), 7.02-7.02 (1H, m), 6.65 (1H, dd), 3.87 (3H, s), 3.72 (3H, s), 2.30 (3H, s).

Present compound 24: ¹H-NMR (CDCl₃) δ: 7.83-7.80 (2H, m), 7.75 (1H, d), 7.66 (1H, d), 7.54-7.51 (2H, m), 7.38 (1H, d), 7.36-7.32 (1H, m), 7.29 (1H, dd), 7.08 (1H, d), 6.63 (1H, d), 3.92 (3H, s), 3.76 (3H, s).

Present compound 25: ¹H-NMR (CDCl₃) δ: 7.78 (1H, s), 7.63 (1H, dd), 7.39 (4H, ddt), 7.27-7.26 (1H, m), 7.04 (1H, d), 6.61 (1H, dd), 3.89 (3H, s), 3.74 (3H, s).

Present compound 26: ¹H-NMR (CDCl₃) δ: 7.78 (1H, s), 7.61 (1H, d), 7.41 (1H, d), 7.30-7.26 (2H, m), 7.23 (1H, d), 7.06 (2H, d), 6.61 (1H, s), 3.91 (3H, s), 3.75 (3H, s).

Present compound 27: ¹H-NMR (CDCl₃) δ: 7.73 (1H, s), 7.49 (1H, d), 6.95 (1H, d), 6.86 (1H, dd), 6.79 (1H, d), 6.54 (1H, dd), 3.87 (3H, s), 3.72 (3H, s), 2.03-1.96 (1H, m), 0.96-0.90 (2H, m), 0.71-0.67 (2H, m).

Present compound 28: ¹H-NMR (CDCl₃) δ: 7.75 (1H, s), 7.53 (1H, d), 6.97 (1H, d), 6.82 (1H, dd), 6.61 (1H, d), 6.57 (1H, d), 4.43 (2H, td), 3.90 (3H, s), 3.74 (3H, s).

Present compound 29: ¹H-NMR (CDCl₃) δ: 7.72 (1H, s), 7.48 (1H, dd), 6.91 (1H, d), 6.78 (1H, dd), 6.55 (1H, d), 6.53 (1H, dd), 4.04 (2H, q), 3.88 (3H, s), 3.72 (3H, s), 1.42 (3H, t).

Present compound 30: ¹H-NMR (CDCl₃) δ: 8.00-7.95 (1H, m), 7.94-7.90 (1H, m), 7.74 (1H, s), 7.72 (1H, dd), 7.39-7.34 (4H, m), 7.28 (1H, br s), 7.10 (1H, d), 6.68 (1H, dd), 3.91 (3H, s), 3.75 (3H, s).

Present compound 31: ¹H-NMR (CDCl₃) δ: 7.78-7.73 (2H, m), 7.23 (1H, d), 7.09 (1H, d), 6.55 (1H, d), 3.90 (3H, s), 3.74 (3H, s).

Present compound 32: ¹H-NMR (CDCl₃) δ: 7.71 (1H, s), 7.45 (1H, d), 6.91 (1H, d), 6.68 (1H, dd), 6.53 (2H, d), 4.68 (1H, s), 3.88 (3H, s), 3.73 (3H, s).

Present compound 33: ¹H-NMR (CDCl₃) δ: 7.66 (1H, s), 7.57 (1H, d), 7.28 (2H, t), 7.05-6.97 (4H, m), 6.88 (1H, dd), 6.75 (1H, d), 6.60 (1H, d), 3.85 (3H, s), 3.72 (3H, s).

Present compound 34: ¹H-NMR (CDCl₃) δ: 7.79 (1H, d), 7.74 (1H, s), 7.18 (1H, d), 7.12 (1H, d), 6.53 (1H, d), 3.87 (3H, s), 3.71 (3H, s), 1.52-1.45 (1H, m), 0.89-0.83 (4H, m).

Present compound 35: ¹H-NMR (CDCl₃) δ: 8.46 (1H, dd), 7.84 (1H, dd), 7.70 (1H, s), 7.64 (1H, d), 7.06 (1H, d), 6.95-6.89 (2H, m), 6.86 (1H, d), 6.63 (1H, dd), 3.88 (3H, s), 3.73 (3H, s).

Present compound 37: ¹H-NMR (CDCl₃) δ: 8.16 (1H, s), 7.76 (1H, s), 7.60 (1H, d), 7.40 (1H, dd), 7.26 (1H, br s), 7.07 (1H, d), 6.61 (1H, dd), 4.23 (2H, q), 3.88 (3H, s), 3.73 (3H, s), 1.33 (3H, t).

Present compound 38: ¹H-NMR (CDCl₃) δ: 8.13 (1H, s), 7.75 (1H, s), 7.59 (1H, d), 7.41 (1H, dd), 7.24 (1H, d), 7.07 (1H, d), 6.60 (1H, dd), 4.49-4.43 (1H, m), 3.87 (3H, s), 3.73 (3H, s), 1.31 (6H, d).

Present compound 39: ¹H-NMR (CDCl₃) δ: 8.16 (1H, s), 7.76 (1H, s), 7.60 (1H, d), 7.40 (1H, dd), 7.26 (1H, br s), 7.07 (1H, d), 6.61 (1H, dd), 4.13 (2H, t), 3.89 (3H, s), 3.73 (3H, s), 1.75 (2H, td), 0.99 (3H, t).

Present compound 40: ¹H-NMR (CDCl₃) δ: 8.21 (1H, s), 7.76 (1H, s), 7.62 (1H, d), 7.37 (1H, dd), 7.26 (1H, br s), 7.11 (1H, d), 6.62 (1H, dd), 5.98 (1H, tt), 4.52 (2H, tt), 3.91 (3H, t), 3.74 (3H, dd).

Present compound 41: ¹H-NMR (CDCl₃) δ: 7.81 (1H, s), 7.62-7.34 (3H, m), 3.94 (3H, s), 3.74 (3H, s).

Present compound 43: ¹H-NMR (CDCl₃) δ: 7.76 (1H, s), 7.59 (1H, d), 7.47 (1H, dd), 7.32 (1H, s), 7.06 (1H, d), 6.59 (1H, dd), 4.25 (2H, q), 3.89 (3H, s), 3.73 (3H, s), 2.28 (3H, s), 1.34 (3H, t).

Present compound 44: ¹H-NMR (CDCl₃) δ: 7.75 (1H, s), 7.59 (1H, d), 7.47 (1H, d), 7.31 (1H, s), 7.05 (1H, d), 6.59 (1H, d), 4.15 (2H, t), 3.89 (3H, s), 3.73 (3H, s), 2.28 (3H, s), 1.75 (2H, dq), 0.99 (3H, t).

Present compound 45: ¹H-NMR (CDCl₃) δ: 7.75 (1H, s), 7.58 (1H, d), 7.49 (1H, dd), 7.31 (1H, d), 7.05 (1H, d), 6.59 (1H, d), 4.49-4.43 (1H, m), 3.89 (3H, s), 3.73 (3H, s), 2.27 (3H, s), 1.31 (6H, d).

Present compound 46: ¹H-NMR (CDCl₃) δ: 7.76 (1H, s), 7.58 (1H, d), 7.47 (1H, dd), 7.31 (1H, s), 7.05 (1H, d), 6.59 (1H, dd), 3.97 (2H, d), 3.89 (3H, s), 3.73 (3H, s), 2.29 (3H, s), 2.12-2.02 (1H, m), 0.97 (6H, d).

Present compound 47: ¹H-NMR (CDCl₃) δ: 7.75 (1H, s), 7.59 (1H, d), 7.47 (1H, dd), 7.46-7.41 (2H, m), 7.39-7.34 (2H, m), 7.33-7.29 (2H, m), 7.06 (1H, d), 6.59 (1H, dd), 5.25 (2H, s), 3.87 (3H, s), 3.73 (3H, s), 2.32 (3H, s).

Present compound 48: ¹H-NMR (CDCl₃) δ: 7.77 (1H, s), 7.61 (1H, d), 7.43 (1H, dd), 7.34 (1H, s), 7.09 (1H, d), 6.61 (1H, dd), 5.98 (1H, tt), 4.55 (2H, tt), 3.90 (3H, s), 3.74 (3H, s), 2.31 (3H, s).

Present compound 49: ¹H-NMR (CDCl₃) δ: 7.77 (1H, s), 7.61 (1H, d), 7.43 (1H, dd), 7.35 (1H, d), 7.09 (1H, d), 6.61 (1H, d), 5.17-4.98 (1H, m), 4.70-4.56 (1H, m), 4.54-4.43 (1H, m), 3.89 (3H, s), 3.74 (3H, s), 2.32 (3H, s).

Present compound 50: ¹H-NMR (CDCl₃) δ: 8.94 (1H, d), 8.05 (1H, ddd), 7.84 (1H, d), 7.71 (1H, dd), 7.63 (1H, dd), 7.34 (1H, dd), 7.25 (1H, t), 6.41 (1H, t), 4.22-4.14 (1H, m), 3.67 (3H, s), 2.96 (3H, d), 2.28 (3H, br s).

Present compound 51: ¹H-NMR (CDCl₃) δ: 7.73 (1H, s), 7.50 (1H, d), 6.97-6.93 (2H, m), 6.87 (1H, s), 6.56 (1H, d), 3.87 (3H, s), 3.72 (3H, s), 2.45 (3H, s).

Present compound 52: ¹H-NMR (CDCl₃) δ: 7.73 (1H, s), 7.52 (1H, d), 7.00-6.95 (2H, m), 6.88 (1H, s), 6.56 (1H, dd), 3.87 (3H, s), 3.72 (3H, s), 2.74 (2H, q), 1.26 (3H, t).

Present compound 53: ¹H-NMR (CDCl₃) δ: 8.22 (1H, s), 7.76 (1H, s), 7.62 (1H, d), 7.37 (1H, dd), 7.27 (1H, d), 7.11 (1H, d), 6.63 (1H, dd), 5.16-4.99 (1H, m), 4.65-4.54 (1H, m), 4.51-4.42 (1H, m), 3.89 (3H, s), 3.74 (3H, s).

Present compound 54: ¹H-NMR (CDCl₃) δ: 8.22 (1H, s), 7.77 (1H, s), 7.62 (1H, d), 7.39 (1H, dd), 7.27-7.26 (1H, m), 7.10 (1H, d), 6.62 (1H, dd), 4.62 (2H, td), 3.90 (3H, s), 3.74 (3H, s).

Present compound 55: ¹H-NMR (CDCl₃) δ: 8.22 (1H, s), 7.75 (1H, s), 7.60 (1H, d), 7.45-7.25 (7H, m), 7.08 (1H, d), 6.61 (1H, dd), 5.22 (2H, s), 3.88 (3H, s), 3.73 (3H, s).

Present compound 56: ¹H-NMR (CDCl₃) δ: 8.17 (1H, s), 7.76 (1H, s), 7.60 (1H, d), 7.40 (1H, dd), 7.26-7.25 (1H, m), 7.07 (1H, d), 6.61 (1H, d), 3.94 (2H, d), 3.89 (3H, s), 3.73 (3H, s), 2.11-2.01 (1H, m), 0.97 (6H, d).

Present compound 57: ¹H-NMR (CDCl₃) δ: 7.72 (1H, s), 7.47 (1H, s), 7.28 (1H, s), 6.97 (1H, d), 6.51 (1H, dd), 3.90 (3H, s), 3.73 (3H, s), 2.47 (3H, s).

Present compound 58: ¹H-NMR (CDCl₃) δ: 7.71 (1H, s), 7.43 (1H, s), 7.01 (1H, d), 6.94 (1H, s), 6.51 (1H, dd), 3.97 (3H, s), 3.87 (3H, s), 3.71 (3H, s), 2.41 (3H, s), 2.20 (3H, s).

Present compound 59: ¹H-NMR (CDCl₃) δ: 7.71 (1H, s), 7.43 (1H, s), 7.00 (1H, d), 6.93 (1H, s), 6.51 (1H, d), 4.22 (2H, q), 3.86 (3H, s), 3.71 (3H, s), 2.42 (3H, s), 2.21 (3H, s), 1.33 (3H, t).

Present compound 60: ¹H-NMR (CDCl₃) δ: 7.71 (1H, s), 7.42 (1H, s), 7.00 (1H, d), 6.93 (1H, s), 6.51 (1H, dd), 4.12 (2H, t), 3.87 (3H, s), 3.71 (3H, s), 2.41 (3H, s), 2.21 (3H, s), 1.74 (2H, tq), 0.98 (3H, t).

Present compound 61: ¹H-NMR (CDCl₃) δ: 7.46 (1H, d), 7.27 (1H, dd), 7.23 (1H, d), 7.18 (1H, t), 6.90 (1H, br s), 6.62 (1H, dd), 4.05 (3H, s), 2.99 (3H, d).

Present compound 62: ¹H-NMR (CDCl₃) δ: 7.72-7.66 (5H, m), 7.41 (1H, dd), 7.32 (1H, d), 7.22 (1H, t), 6.92 (1H, d), 6.69 (1H, dd), 4.06 (3H, s), 3.00 (3H, d).

Present compound 63: ¹H-NMR (CDCl₃) δ: 7.81 (1H, s), 7.63 (1H, d), 7.50 (1H, s), 7.41 (1H, dd), 7.30 (1H, d), 3.96 (3H, s), 3.77 (3H, s).

Present compound 64: ¹H-NMR (CDCl₃) δ: 7.89 (1H, s), 7.53 (1H, dd), 7.32 (1H, dd), 7.29-7.28 (1H, m), 7.23 (1H, d), 3.97 (3H, s), 3.77 (3H, s).

Present compound 65: ¹H-NMR (CDCl₃) δ: 7.84 (1H, s), 7.36 (1H, d), 7.18-7.13 (2H, m), 6.31 (1H, t), 3.89 (3H, s), 3.72 (3H, s), 2.22 (3H, br s).

Present compound 66: ¹H-NMR (CDCl₃) δ: 7.88-7.83 (2H, m), 7.83 (1H, s), 7.78 (1H, d), 7.64-7.56 (3H, m), 7.54 (1H, dd), 7.29 (1H, dd), 3.96 (3H, s), 3.78 (3H, s).

Present compound 67: ¹H-NMR (CDCl₃) δ: 7.88-7.83 (2H, m), 7.73-7.69 (4H, m), 7.59 (1H, s), 7.55 (1H, dd), 7.31 (1H, d), 3.95 (3H, s), 3.78 (3H, s).

Present compound 68: ¹H-NMR (CDCl₃) δ: 7.92 (1H, s), 7.73 (1H, dd), 7.38 (1H, dd), 7.32-7.28 (3H, m), 7.19 (1H, t), 7.13 (1H, d), 3.97 (3H, s), 3.78 (3H, s).

Present compound 69: ¹H-NMR (CDCl₃) δ: 7.91 (1H, s), 7.69 (1H, dd), 7.53 (2H, d), 7.44 (1H, dd), 7.27 (1H, d), 7.22-7.21 (1H, m), 6.96 (2H, d), 4.63-4.57 (1H, m), 3.96 (3H, s), 3.76 (3H, s), 1.37 (6H, d).

Present compound 70: ¹H-NMR (CDCl₃) δ: 8.96 (1H, d), 8.05 (1H, dd), 7.89 (1H, s), 7.71 (1H, d), 7.63 (1H, d), 7.33 (1H, dd), 7.20 (1H, s), 6.41 (1H, s), 3.90 (3H, s), 3.74 (3H, s), 2.29 (3H, br s).

Present compound 71: ¹H-NMR (CDCl₃) δ: 7.87 (1H, s), 7.60 (2H, d), 7.56 (1H, d), 7.34 (2H, d), 7.30 (1H, dd), 7.17 (1H, t), 6.36 (1H, t), 3.88 (3H, s), 3.72 (3H, s), 2.27 (3H, br s), 1.74 (2H, dd), 1.44 (2H, dd).

Present compound 72: ¹H-NMR (CDCl₃) δ: 7.48 (1H, d), 7.29 (1H, dd), 7.19-7.16 (2H, m), 6.64 (1H, dd), 4.14 (3H, s), 3.95 (3H, s).

Present compound 73: ¹H-NMR (CDCl₃) δ: 8.94 (1H, d), 8.05 (1H, ddd), 7.84 (1H, d), 7.71 (1H, dd), 7.63 (1H, d), 7.34 (1H, dd), 7.26 (1H, d), 6.41 (1H, t), 4.18 (1H, m), 3.67 (3H, s), 2.96 (3H, d), 2.28 (3H, br s).

Present compound 74: ¹H-NMR (CDCl₃) δ: 7.77 (1H, s), 7.67-7.63 (1H, m), 7.57-7.53 (2H, m), 7.49-7.45 (2H, m), 7.42-7.38 (2H, m), 7.36-7.32 (2H, m), 7.21 (1H, s), 7.07-7.03 (3H, m), 6.62 (1H, d), 5.12 (2H, s), 3.89 (3H, s), 3.73 (3H, s).

Present compound 75: ¹H-NMR (CDCl₃) δ: 7.77 (1H, s), 7.69-7.61 (2H, m), 7.50-7.30 (9H, m), 7.05 (1H, d), 6.93 (1H, dd), 6.63 (1H, dd), 5.13 (2H, s), 3.88 (3H, s), 3.73 (3H, s).

Present compound 76: ¹H-NMR (CDCl₃) δ: 8.13 (1H, s), 7.84 (1H, s), 7.79 (1H, s), 7.73 (2H, s), 7.65 (1H, d), 7.34 (1H, d), 7.25 (1H, s), 7.04 (1H, d), 6.63 (1H, d), 3.91 (3H, s), 3.75 (3H, s).

Present compound 77: ¹H-NMR (CDCl₃) δ: 7.72 (1H, s), 7.65 (1H, d), 7.57 (1H, s), 7.48 (1H, d), 7.33 (1H, d), 7.12 (1H, dd), 7.07 (1H, d), 7.04 (1H, s), 6.65 (1H, d), 3.88 (3H, s), 3.78 (3H, d), 3.73 (3H, s), 2.35 (3H, s), 2.16 (3H, s).

Present compound 78: ¹H-NMR (CDCl₃) δ: 7.92 (1H, d), 7.80 (1H, s), 7.74 (2H, d), 7.66 (2H, m), 7.64 (1H, m), 7.43 (2H, d), 7.06 (1H, d), 6.80 (1H, d), 6.63 (1H, dd), 3.90 (3H, s), 3.74 (3H, s).

Present compound 79: ¹H-NMR (CDCl₃) δ: 7.46 (1H, d), 7.30-7.28 (2H, m), 7.18 (1H, d), 6.61 (1H, dd), 4.50 (2H, dd), 4.23 (2H, dd), 4.07 (3H, s).

The Present compounds 36 to 40, 42 to 49, 53 to 56, and 58 to 60 are the Compounds of the present invention.

Examples of the Present compound prepared according to the above Production methods and Preparation Examples are shown below.

The compound represented by formula (I), wherein X¹ represents —C(H)═, X² represents —C(O)OCH₃, Y¹ represents —N═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX1”).

The compound represented by formula (I), wherein X¹ represents —C(H)═, X² represents —C(O)OCH₃, Y¹ represents —N═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX2”).

The compound represented by formula (I), wherein X¹ represents —C(H)═, X² represents —C(O)OCH₃, Y¹ represents —N═, R¹ represents a bromine atom, R² represents a hydrogen atom, R³ represents a hydrogen atom, Y² represents —C(H)═, and E represents any one substituent described in Table V1 to Table V7 (hereinafter referred to as “Compound group SX3”).

The compound represented by formula (I), wherein X¹ represents —C(H)═, X² represents —C(O)OCH₃, Y¹ represents —N═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX4”).

The compound represented by formula (I), wherein X¹ represents —C(H)═, X² represents —C(O)OCH₃, Y¹ represents —C(H)═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX5”).

The compound represented by formula (I), wherein X¹ represents —C(H)═, X² represents —C(O)OCH₃, Y¹ represents —C(H)═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX6”).

The compound represented by formula (I), wherein X¹ represents —C(H)═, X² represents —C(O)OCH₃, Y¹ represents —C(R⁴)═, R¹ and R⁴ are combined with the carbon atoms to which they are attached to form a benzene ring, R² represents a hydrogen atom, R³ represents a hydrogen atom, Y² represents —C(H)═, and E represents any one substituent described in Table V1 to Table V7 (hereinafter referred to as “Compound group SX7”).

The compound represented by formula (I), wherein X¹ represents —C(H)═, X² represents —C(O)OCH₃, Y¹ represents —C(H)═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX8”).

The compound represented by formula (I), wherein X¹ represents —C(H)═, X² represents —C(O)NHCH₃, Y¹ represents —N═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX9”).

The compound represented by formula (I), wherein X¹ represents —C(H)═, X² represents —C(O)NHCH₃, Y¹ represents —N═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX10”).

The compound represented by formula (I), wherein X¹ represents —C(H)═, X² represents —C(O)NHCH₃, Y¹ represents —N═, R¹ represents a bromine atom, R² represents a hydrogen atom, R³ represents a hydrogen atom, Y² represents —C(H)═, and E represents any one substituent described in Table V1 to Table V7 (hereinafter referred to as “Compound group SX11”).

The compound represented by formula (I), wherein X¹ represents —C(H)═, X² represents —C(O)NHCH₃, Y¹ represents —N═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX12”).

The compound represented by formula (I), wherein X¹ represents —C(H)═, X² represents —C(O)NHCH₃, Y¹ represents —C(H)═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX13”).

The compound represented by formula (I), wherein X¹ represents —C(H)═, X² represents —C(O)NHCH₃, Y¹ represents —C(H)═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX14”).

The compound represented by formula (I), wherein X¹ represents —C(H)═, X² represents —C(O)NHCH₃, Y¹ represents —C(R⁴)═, R¹ and R⁴ are combined with the carbon atoms to which they are attached to form a benzene ring, R² represents a hydrogen atom, R³ represents a hydrogen atom, Y² represents —C(H)═, and E represents any one substituent described in Table V1 to Table V7 (hereinafter referred to as “Compound group SX15”).

The compound represented by formula (I), wherein X¹ represents —C(H)═, X² represents —C(O)NHCH₃, Y¹ represents —C(H)═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX16”).

The compound represented by formula (I), wherein X¹ represents —N═, X² represents —C(O)OCH₃, Y¹ represents —N═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX17”).

The compound represented by formula (I), wherein X¹ represents —N═, X² represents —C(O)OCH₃, Y¹ represents —N═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX18”).

The compound represented by formula (I), wherein X¹ represents —N═, X² represents —C(O)OCH₃, Y¹ represents —N═, R¹ represents a bromine atom, R² represents a hydrogen atom, R³ represents a hydrogen atom, Y² represents —C(H)═, and E represents any one substituent described in Table V1 to Table V7 (hereinafter referred to as “Compound group SX19”).

The compound represented by formula (I), wherein X¹ represents —N═, X² represents —C(O)OCH₃, Y¹ represents —N═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX20”).

The compound represented by formula (I), wherein X¹ represents —N═, X² represents —C(O)OCH₃, Y¹ represents —C(H)═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX21”).

The compound represented by formula (I), wherein X¹ represents —N═, X² represents —C(O)OCH₃, Y¹ represents —C(H)═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX22”).

The compound represented by formula (I), wherein X¹ represents —N═, X² represents —C(O)OCH₃, Y¹ represents —C(R⁴)═, R¹ and R⁴ are combined with the carbon atoms to which they are attached to form a benzene ring, R² represents a hydrogen atom, R³ represents a hydrogen atom, Y² represents —C(H)═, and E represents any one substituent described in Table V1 to Table V7 (hereinafter referred to as “Compound group SX23”).

The compound represented by formula (I), wherein X¹ represents —N═, X² represents —C(O)OCH₃, Y¹ represents —C(H)═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX24”).

The compound represented by formula (I), wherein X¹ represents —N═, X² represents —C(O)NHCH₃, Y¹ represents —N═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX25”).

The compound represented by formula (I), wherein X¹ represents —N═, X² represents —C(O)NHCH₃, Y¹ represents —N═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX26”).

The compound represented by formula (I), wherein X¹ represents —N═, X² represents —C(O)NHCH₃, Y¹ represents —N═, R¹ represents a bromine atom, R² represents a hydrogen atom, R³ represents a hydrogen atom, Y² represents —C(H)═, and E represents any one substituent described in Table V1 to Table V7 (hereinafter referred to as “Compound group SX27”).

The compound represented by formula (I), wherein X¹ represents —N═, X² represents —C(O)NHCH₃, Y¹ represents —N═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX28”).

The compound represented by formula (I), wherein X¹ represents —N═, X² represents —C(O)NHCH₃, Y¹ represents —C(H)═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX29”).

The compound represented by formula (I), wherein X¹ represents —N═, X² represents —C(O)NHCH₃, Y¹ represents —C(H)═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX30”).

The compound represented by formula (I), wherein X¹ represents —N═, X² represents —C(O)NHCH₃, Y¹ represents —C(R⁴)═, R¹ and R⁴ are combined with the carbon atoms to which they are attached to form a benzene ring, R² represents a hydrogen atom, R³ represents a hydrogen atom, Y² represents —C(H)═, and E represents any one substituent described in Table V1 to Table V7 (hereinafter referred to as “Compound group SX31”).

The compound represented by formula (I), wherein X¹ represents —N═, X² represents —C(O)NHCH₃, Y¹ represents —C(H)═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX32”).

The compound represented by formula (I), wherein X¹ represents —N═, X² represents a 5,6-dihydro-1,4,2-dioxazin-3-yl group, Y¹ represents —N═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX33”).

The compound represented by formula (I), wherein X¹ represents —N═, X² represents a 5,6-dihydro-1,4,2-dioxazin-3-yl group, Y¹ represents —N═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX34”).

The compound represented by formula (I), wherein X¹ represents —N═, X² represents a 5,6-dihydro-1,4,2-dioxazin-3-yl group, Y¹ represents —N═, R¹ represents a bromine atom, R² represents a hydrogen atom, R³ represents a hydrogen atom, Y² represents —C(H)═, and E represents any one substituent described in Table V1 to Table V7 (hereinafter referred to as “Compound group SX35”).

The compound represented by formula (I), wherein X¹ represents —N═, X² represents a 5,6-dihydro-1,4,2-dioxazin-3-yl group, Y¹ represents —N═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX36”).

The compound represented by formula (I), wherein X¹ represents —N═, X² represents a 5,6-dihydro-1,4,2-dioxazin-3-yl group, Y¹ represents —C(H)═, R¹ represents a hydrogen atom, R² represents a hydrogen atom, R³ represents a hydrogen atom, Y² represents —C(H)═, and E represents any one substituent described in Table V1 to Table V7 (hereinafter referred to as “Compound group SX37”).

The compound represented by formula (I), wherein X¹ represents —N═, X² represents a 5,6-dihydro-1,4,2-dioxazin-3-yl group, Y¹ represents —C(H)═, R¹ represents a chlorine atom, R² represents a hydrogen atom, R³ represents a hydrogen atom, Y² represents —C(H)═, and E represents any one substituent described in Table V1 to Table V7 (hereinafter referred to as “Compound group SX38”).

The compound represented by formula (I), wherein X¹ represents —N═, X² represents a 5,6-dihydro-1,4,2-dioxazin-3-yl group, Y¹ represents —C(R⁴)═, R¹ and R⁴ are combined with the carbon atoms to which they are attached to form a benzene ring, R² represents a hydrogen atom, R³ represents a hydrogen atom, Y² represents —C(H)═, and E represents any one substituent described in Table V1 to Table V7 (hereinafter referred to as “Compound group SX39”).

The compound represented by formula (I), wherein X¹ represents —N═, X² represents a 5,6-dihydro-1,4,2-dioxazin-3-yl group, Y¹ represents —C(H)═, R¹ represents a hydrogen atom, R² represents a hydrogen atom, R³ represents a hydrogen atom, Y² represents —N═, and E represents any one substituent described in Table V1 to Table V7 (hereinafter referred to as “Compound group SX40”).

Next, Formulation Examples are shown below. The “part(s)” in Formulation Examples represents “part(s) by weight”. Also, the expression of “Present compound S” represents the compounds described in the Compound groups SX1 to SX40.

### Formulation Example 1

A mixture of polyoxyethylene alkyl ether sulfate ammonium salt and silica (weight ratio of 1:1) (35 parts), any one of the Present compound S (10 parts), and water (55 parts) are mixed, and the resulting mixture is subjected to fine grinding according to a wet grinding method to obtain each formulation.

### Formulation Example 2

Any one of the Present compound S (50 parts), calcium lignin sulfonate (3 parts), sodium lauryl sulfate (2 parts), and silica (45 parts) are ground and mixed to obtain each formulation.

### Formulation Example 3

Any one of the Present compound S (5 parts), polyoxyethylene styryl phenyl ether (9 parts), polyoxyethylene decyl ether (number of added ethylene-oxide: 5) (5 parts), calcium dodecylbenzene sulfonate (6 parts), and xylene (75 parts) are mixed to obtain each formulation.

### Formulation Example 4

Any one of the Present compound S (2 parts), silica (1 part), calcium lignin sulfonate (2 parts), bentonite (30 parts), and kaolin clay (65 parts) are ground and mixed, an appropriate amount of water is added thereto, the resulting mixture is kneaded, subjected to granulation with a granulator, and then dried to obtain each formulation.

Next, Test Examples are shown below.

### Test Example 1

A true leaf of a soybean (cultivar: Kurosengoku) was cut into a diameter of 1 cm to prepare each leaf disc. To each well of a 24 well microplate was dispensed 1 mL of an agar medium (agar concentration 1.2%), and then one of said leaf disc was placed on each well. To a mixture of Sorpol (registered trademark) 1200KX (0.5 μL), DMSO (4.5 μL), and xylene (5 μL) was added a DMSO solution (20 μL) comprising a test compound (10000 ppm), and the resulting mixture was mixed. The resulting mixture was diluted with ion exchange water to prepare a spray solution comprising a prescribed concentration of the test compound. Said spray solution was sprayed into each leaf disc at a ratio of 10 μL per leaf disc. After 1 day, an aqueous suspension of spores of soybean rust fungus (Phakopsora pachyrhizi) having an amino acid substitution of F129L in a mitochondrial cytochrome b protein (1.0×10⁵/mL) was inoculated by spraying on each leaf disc. After the inoculation, the microplate was placed into an artificial climate chamber (Lighting: 6 hours, Lights-out: 18 hours, Temperature: 23° C., Humidity: 60%). After 1 day, the leaf discs were air-dried until water droplets on the surfaces of the leaf discs disappeared, and the microplate was placed into the artificial climate chamber again for 12 days. Then, lesion area of soybean rust was investigated. As a result, when the prescribed concentration was 50 ppm, each lesion area of leaf disc treated with any one of the Present compound 2 to 13, 15 to 30, 33 to 40, 42 to 49, 52 to 56, 58, 60, 65, 66, or 74 to 79 as the test compound was 30% or less relative to the lesion area of non-treated leaf disc. Here, the term of “non-treated” means that a spray solution comprising a test compound was not sprayed into a leaf disc.

### Test Example 2

Each test is carried out according to the Test Example 1 by using any one of the Present compound S as the test compound with a prescribed concentration of 12.5 ppm. As a result, control effects on soybean rust can be confirmed in the leaf discs treated with test compounds.

### Test Example 3

Each test is carried out according to the Test Example 1 by using any one of the Present compound S as the test compound with a prescribed concentration of 3.1 ppm. As a result, control effects on soybean rust can be confirmed in the leaf discs treated with test compounds.

### Comparative Test Example 1

Each test was carried out according to the Test Example 1 by using the Present compound 4, 3, 11, or 42, or azoxystrobin, dimoxystrobin, or metominostrobin as the test compound with a prescribed concentration of 50 ppm. The results are shown in Table A.

TABLE A

| | Lesion area |
|---|---|
| | (%) at the |
| | concentration |
| Compound | of 50 ppm |
| 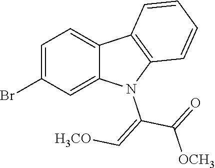 [SMILES: CO/C=C(/C(=O)OC)n1c2ccccc2c2ccc(Br)cc21] | 0 |
| Present compound 4 | |
| 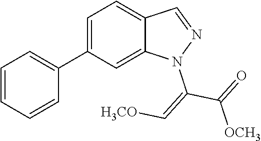 [SMILES: CO/C=C(/C(=O)OC)n1ncc2ccc(-c3ccccc3)cc21] | 0 |
| Present compound 3 | |
| 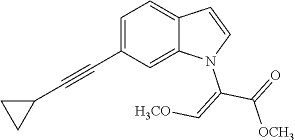 [SMILES: CO/C=C(/C(=O)OC)n1ccc2ccc(C#CC3CC3)cc21] | 0 |
| Present compound 11 | |
| 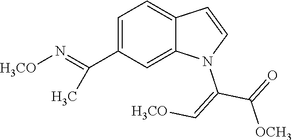 [SMILES: CO/C=C(/C(=O)OC)n1ccc2ccc(/C(C)=N/OC)cc21] | 0 |
| Present compound 42 | |
| 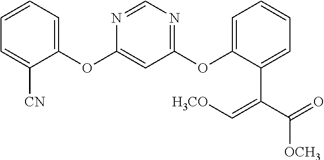 [SMILES: CO/C=C(/C(=O)OC)c1ccccc1Oc1cc(Oc2ccccc2C#N)ncn1] | 100 |
| Azoxystrobin | |
| 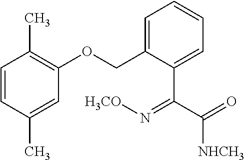 [SMILES: CNC(=O)/C(=N/OC)c1ccccc1COc1cc(C)ccc1C] | 100 |
| Dimoxystrobin | |
| 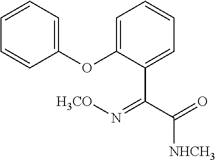 [SMILES: CNC(=O)/C(=N/OC)c1ccccc1Oc1ccccc1] | 100 |
| Metominostrobin | |

The above results demonstrate that the Present compound has excellent activities against soybean rust fungi having an amino acid substitution of F129L as compared to several commercially available QoI fungicides.

### INDUSTRIAL APPLICABILITY

The Present compound can be used for controlling soybean rust fungi having an amino acid substitution of F129L in a mitochondrial cytochrome b protein.

1. A method for controlling a soybean rust fungus having an amino acid substitution of F129L in a mitochondrial cytochrome b protein, which comprises applying an effective amount of a compound represented by formula (I) 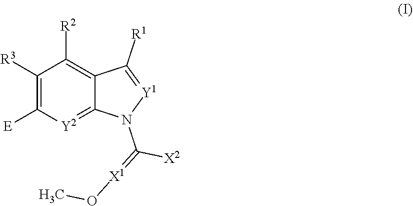
[wherein:
X¹ represents —C(H)═ or —N═;
X² represents —C(O)OCH₃, —C(O)NHCH₃, or a 5,6-dihydro-1,4,2-dioxazin-3-yl group;
Y¹ represents —C(R⁴)═ or —N═;
Y² represents —C(R⁵)═ or —N═;
R¹ and R⁴ are identical to or different from each other, and each represent a C1-C4 alkyl group, a C1-C4 alkoxy group, a C2-C4 alkylcarbonyl group, a C2-C4 alkoxycarbonyl group, a (C1-C3 alkylamino)carbonyl group, a (C2-C6 dialkylamino)carbonyl group {wherein said C1-C4 alkyl group, said C1-C4 alkoxy group, said C2-C4 alkylcarbonyl group, said C2-C4 alkoxycarbonyl group, said (C1-C3 alkylamino)carbonyl group, and said (C2-C6 dialkylamino)carbonyl group are optionally substituted with one or more halogen atom(s)}, a formyl group, an aminocarbonyl group, a cyano group, a nitro group, a halogen atom, a hydroxy group, or a hydrogen atom;
R², R³, and R⁵ are identical to or different from each other, and each represent a C1-C4 alkyl group, a C1-C4 alkoxy group {wherein said C1-C4 alkyl group and said C1-C4 alkoxy group are optionally substituted with one or more halogen atom(s)}, a cyano group, a nitro group, a halogen atom, a hydroxy group, or a hydrogen atom;
when Y¹ represents —C(R⁴)═, R¹ and R⁴ are optionally combined with the carbon atoms to which they are attached to form a C5-C6 nonaromatic hydrocarbon ring, a 5-6 membered nonaromatic heterocycle {wherein said C5-C6 nonaromatic hydrocarbon ring and said 5-6 membered nonaromatic heterocycle are optionally substituted with one or more substituent(s) selected from Group B}, a benzene ring, or a 5-6 membered aromatic heterocycle {wherein said benzene ring and said 5-6 membered aromatic heterocycle are optionally substituted with one or more substituent(s) selected from Group C};
E represents a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group D}, R⁹-L¹-CH₂—, R¹⁰R¹¹C═NO—CH₂—, R¹²O—N═C(R¹³)—C(R¹⁴)═NO—CH₂—, R¹⁵C(O)—C(R¹⁶)═NO—CH₂—, R¹⁷R¹⁸N—C(S)—O—CH₂—, R¹⁹N═C(R²⁰)—S—CH₂—, R²¹N═C(SR²²)—S—CH₂—, R²³O—N═C(R²⁴)—S—CH₂—, R²⁵O—N═C(SR²⁶)—S—CH₂—, R²⁷O—N═C(R²⁸)—, R²⁹R³⁶C═N—N═C(R³¹)—, R³²R³³N—N═C(R³⁴)—, R³⁵—N═C(R³⁶)—, R³⁷SC(R³⁸)═N—, R³⁹SC(SR⁴⁰)═N—, R⁴¹L²-, R⁴³C(O)O—, R⁴⁴OC(O)O—, R⁴⁵R⁴⁶NC(O)O—, R⁴⁷R⁴⁸NC(S)O—, R⁴⁹S(O)₂O—, R⁵⁰R⁵¹NS(O)₂O—, a cyano group, a nitro group, a hydroxy group, or a halogen atom;
L¹ and L² are identical to or different from each other, and each represent an oxygen atom or a sulfur atom;
R⁹ represents a C6-C10 aryl group or a 5-10 membered aromatic heterocyclic group {wherein said C6-C10 aryl group and said 5-10 membered aromatic heterocyclic group are optionally substituted with one or more substituent(s) selected from Group D};
R¹², R¹⁵, R¹⁷, R¹⁹, R²¹, R²³, R²⁵, R²⁹, R³², R³⁷, R³⁹, R⁴³, R⁴⁴, R⁴⁵, R⁴⁷, R⁴⁹, and R⁵⁰ are identical to or different from each other, and each represent a C1-C6 chain hydrocarbon group optionally substituted with one or more substituent(s) selected from Group F, a C3-C10 alicyclic hydrocarbon group optionally substituted with one or more substituent(s) selected from Group B, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group C};
R¹⁰, R²⁷, R³⁵, and R⁴¹ are identical to or different from each other, and each represent a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group D};
R¹¹, R¹³, R¹⁴, R¹⁶, R¹⁸, R²⁰, R²², R²⁴, R²⁶, R²⁸, R³⁰, R³¹, R³³, R³⁴, R³⁶, R³⁸, R⁴⁰, R⁴⁶, R⁴⁸, and R⁵¹ are identical to or different from each other, and each represent a C1-C3 chain hydrocarbon group optionally substituted with one or more halogen atom(s), a cyclopropyl group, or a hydrogen atom;
R¹⁰ and R¹¹ are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}; and
R²⁹ and R³⁰ are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B};
Group A: a group consisting of a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C1-C4 alkoxy group, a C1-C4 alkylthio group {wherein said C1-C4 alkoxy group and said C1-C4 alkylthio group are optionally substituted with one or more substituent(s) selected from Group F}, a halogen atom, a cyano group, a nitro group, a hydroxy group, an oxo group, a thioxo group, a C6-C10 aryl group, and a 5-10 membered aromatic heterocyclic group {wherein said C6-C10 aryl group and said 5-10 membered aromatic heterocyclic group are optionally substituted with one or more substituent(s) selected from Group D};
Group B: a group consisting of an oxo group, a thioxo group, a C1-C3 chain hydrocarbon group, a C1-C3 alkoxy group {wherein said C1-C3 chain hydrocarbon group and said C1-C3 alkoxy group are optionally substituted with one or more halogen atom(s)}, a halogen atom, and a cyano group;
Group C: a group consisting of a C1-C6 chain hydrocarbon group, a C1-C6 alkoxy group, a C1-C6 alkylthio group {wherein said C1-C6 chain hydrocarbon group, said C1-C6 alkoxy group, and said C1-C6 alkylthio group are optionally substituted with one or more substituent(s) selected from Group F}, a C3-C6 cycloalkyl group {wherein said C3-C6 cycloalkyl group is optionally substituted with one or more substituent(s) selected from Group B}, a cyano group, a nitro group, a halogen atom, and a hydroxy group;
Group D: a group consisting of a C1-C6 chain hydrocarbon group, a C1-C6 alkoxy group, a C1-C6 alkylthio group, a C1-C6 alkylamino group, a C2-C8 dialkylamino group, a (C1-C6 alkyl)carbonyl group, a (C1-C6 alkoxy)carbonyl group, a (C1-C6 alkylamino)carbonyl group, a (C2-C8 dialkylamino)carbonyl group {wherein said C1-C6 chain hydrocarbon group, said C1-C6 alkoxy group, said C1-C6 alkylthio group, said C1-C6 alkylamino group, said C2-C8 dialkylamino group, said (C1-C6 alkyl)carbonyl group, said (C1-C6 alkoxy)carbonyl group, said (C1-C6 alkylamino)carbonyl group, and said (C2-C8 dialkylamino)carbonyl group are optionally substituted with one or more substituent(s) selected from Group F}, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group C}, a halogen atom, a cyano group, a nitro group, a hydroxy group, and an amino group;
Group F: a group consisting of a C3-C4 cycloalkyl group, a halogen atom, and a C1-C3 alkoxy group]
or an N-oxide or an agriculturally acceptable salt thereof to a soybean or soil for cultivating a soybean.

2. The method according to claim 1, wherein
X¹ represents —C(H)═; and
X² represents —C(O)OCH₃
in the compound represented by formula (I), or an N-oxide or an agriculturally acceptable salt thereof.

3. The method according to claim 1, wherein
E represents a C1-C6 alkyl group optionally substituted with one or more substituent(s) selected from Group A, a C3-C6 cycloalkyl group {wherein said C3-C6 cycloalkyl group is optionally substituted with one or more substituent(s) selected from the group consisting of a C1-C3 alkyl group and a halogen atom}, a phenyl group, a 5-6 membered aromatic heterocyclic group {wherein said phenyl group and said 5-6 membered aromatic heterocyclic group are optionally substituted with one or more substituent(s) selected from Group D}, R⁹-L¹-CH₂—, R⁴¹L²-, or a halogen atom
in the compound represented by formula (I), or an N-oxide or an agriculturally acceptable salt thereof.

4. (canceled)

5. A compound represented by formula (II) 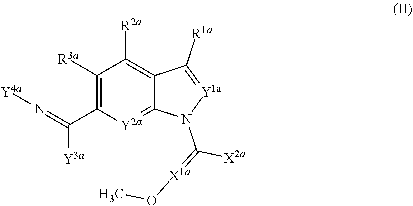
[wherein:
X^{1a} represents —C(H)═ or —N═;
X^{2a} represents —C(O)OCH₃, —C(O)NHCH₃, or a 5,6-dihydro-1,4,2-dioxazin-3-yl group;
Y^{1a} represents —C(R^{4a})═ or —N═;
Y^{2a} represents —C(R^{5a})═ or —N═;
R^{1a} and R^{4a} are identical to or different from each other, and each represent a C1-C4 alkyl group, a C1-C4 alkoxy group, a C2-C4 alkylcarbonyl group, a C2-C4 alkoxycarbonyl group, a (C1-C3 alkylamino)carbonyl group, a (C2-C6 dialkylamino)carbonyl group {wherein said C1-C4 alkyl group, said C1-C4 alkoxy group, said C2-C4 alkylcarbonyl group, said C2-C4 alkoxycarbonyl group, said (C1-C3 alkylamino)carbonyl group, and said (C2-C6 dialkylamino)carbonyl group are optionally substituted with one or more halogen atom(s)}, a formyl group, an aminocarbonyl group, a cyano group, a nitro group, a halogen atom, a hydroxy group, or a hydrogen atom;
R^{2a}, R^{3a}, and R^{5a} are identical to or different from each other, and each represent a C1-C4 alkyl group, a C1-C4 alkoxy group {wherein said C1-C4 alkyl group and said C1-C4 alkoxy group are optionally substituted with one or more halogen atom(s)}, a cyano group, a nitro group, a halogen atom, a hydroxy group, or a hydrogen atom;
when Y^{1a} represents —C(R^{4a})═, R^{1a} and R^{4a} are optionally combined with the carbon atoms to which they are attached to form a C5-C6 nonaromatic hydrocarbon ring, a 5-6 membered nonaromatic heterocycle {wherein said C5-C6 nonaromatic hydrocarbon ring and said 5-6 membered nonaromatic heterocycle are optionally substituted with one or more substituent(s) selected from Group B^{a}}, a benzene ring, or a 5-6 membered aromatic heterocycle {wherein said benzene ring and said 5-6 membered aromatic heterocycle are optionally substituted with one or more substituent(s) selected from Group C^{a}};
Y^{3a} represents a C1-C3 chain hydrocarbon group optionally substituted with one or more halogen atom(s), a cyclopropyl group, or a hydrogen atom;
Y^{4a} represents R^{27a}O—, R^{29a}R^{30a}═N—, R^{32a}R^{33a}N—, or R^{35a}—;
R^{29a} and R^{32a} are identical to or different from each other, and each represent a C1-C6 chain hydrocarbon group optionally substituted with one or more substituent(s) selected from Group F^{a}, a C3-C10 alicyclic hydrocarbon group optionally substituted with one or more substituent(s) selected from Group B^{a}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group C^{a}};
R^{27a} and R^{35a} are identical to or different from each other, and each represent a C1-C6 chain hydrocarbon group optionally substituted with one or more substituent(s) selected from Group A^{a}, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B^{a}}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group D^{a}};
R^{30a} and R^{33a} are identical to or different from each other, and each represent a C1-C3 chain hydrocarbon group optionally substituted with one or more halogen atom(s), a cyclopropyl group, or a hydrogen atom;
R^{29a} and R^{30a} are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B^{a}};
Group A^{a}: a group consisting of a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B^{a}}, a C1-C4 alkoxy group, a C1-C4 alkylthio group {wherein said C1-C4 alkoxy group and said C1-C4 alkylthio group are optionally substituted with one or more substituent(s) selected from Group F^{a}}, a halogen atom, a cyano group, a nitro group, a hydroxy group, an oxo group, a thioxo group, a C6-C10 aryl group, and a 5-10 membered aromatic heterocyclic group {wherein said C6-C10 aryl group and said 5-10 membered aromatic heterocyclic group are optionally substituted with one or more substituent(s) selected from Group D^{a}};
Group B^{a}: a group consisting of an oxo group, a thioxo group, a C1-C3 chain hydrocarbon group, a C1-C3 alkoxy group {wherein said C1-C3 chain hydrocarbon group and said C1-C3 alkoxy group are optionally substituted with one or more halogen atom(s)}, a halogen atom, and a cyano group;
Group C^{a}: a group consisting of a C1-C6 chain hydrocarbon group, a C1-C6 alkoxy group, a C1-C6 alkylthio group {wherein said C1-C6 chain hydrocarbon group, said C1-C6 alkoxy group, and said C1-C6 alkylthio group are optionally substituted with one or more substituent(s) selected from Group F^{a}}, a C3-C6 cycloalkyl group {wherein said C3-C6 cycloalkyl group is optionally substituted with one or more substituent(s) selected from Group B^{a}}, a cyano group, a nitro group, a halogen atom, and a hydroxy group;
Group D^{a}: a group consisting of a C1-C6 chain hydrocarbon group, a C1-C6 alkoxy group, a C1-C6 alkylthio group, a C1-C6 alkylamino group, a C2-C8 dialkylamino group, a (C1-C6 alkyl)carbonyl group, a (C1-C6 alkoxy)carbonyl group, a (C1-C6 alkylamino)carbonyl group, a (C2-C8 dialkylamino)carbonyl group {wherein said C1-C6 chain hydrocarbon group, said C1-C6 alkoxy group, said C1-C6 alkylthio group, said C1-C6 alkylamino group, said C2-C8 dialkylamino group, said (C1-C6 alkyl)carbonyl group, said (C1-C6 alkoxy)carbonyl group, said (C1-C6 alkylamino)carbonyl group, and said (C2-C8 dialkylamino)carbonyl group are optionally substituted with one or more substituent(s) selected from Group F^{a}}, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B^{a}}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group C^{a}}, a halogen atom, a cyano group, a nitro group, a hydroxy group, and an amino group;
Group F^{a}: a group consisting of a C3-C4 cycloalkyl group, a halogen atom, and a C1-C3 alkoxy group]
or an N-oxide thereof, or a salt thereof.

6. The compound or an N-oxide thereof, or a salt thereof according to claim 5, wherein Y^{4a} represents R^{27a}O—.

7. An agrochemical composition comprising the compound or an N-oxide thereof, or a salt thereof according to claim 5, and an inert carrier.

8. A composition comprising one or more ingredient(s) selected from the group consisting of Group (a), Group (b), Group (c), and Group (d), and the compound or an N-oxide thereof, or a salt thereof according to claim 5:
Group (a): a group consisting of insecticidal active ingredients, miticidal active ingredients, and nematicidal active ingredients;
Group (b): fungicidal active ingredients;
Group (c): plant growth regulatory ingredients;
Group (d): repellent ingredients.

9. A method for controlling a soybean rust fungus having an amino acid substitution of F129L in a mitochondrial cytochrome b protein, which comprises applying an effective amount of the composition according to claim 7 to a soybean or soil for cultivating a soybean.

10. (canceled)

11. A seed holding an effective amount of the compound or an N-oxide thereof, or a salt thereof according to claim 5.

12. A seed holding an effective amount of the composition according to claim 7.
